(19) United States
(12) Patent Application Publication
(10) Pub. No.: US 2025/0115886 A1
(43) Pub. Date: Apr. 10, 2025

(54) DESIGNER MEMBRANELESS ORGANELLES SEQUESTER NATIVE FACTORS FOR CONTROL OF CELL BEHAVIOR

(71) Applicant: THE TRUSTEES OF THE UNIVERSITY OF PENNSYLVANIA, Philadelphia, PA (US)

(72) Inventors: Matthew C. Good, Philadelphia, PA (US); Mikael V. Garabedian, Bala Cynwyd, PA (US); Wentao Wang, Philadelphia, PA (US)

(21) Appl. No.: 18/293,711

(22) PCT Filed: Aug. 2, 2022
(86) PCT No.: PCT/US22/74438

§ 371 Date: Jan. 30, 2024

Related U.S. Application Data
(60) Provisional application No. 63/228,484, filed on Aug. 2, 2021.

(51) Int. Cl. C12N 9/14; C07K 14/47; C12N 15/86

(52) U.S. Cl. CPC C12N 9/14; C07K 14/47; C12N 15/86; C07K 2319/60; C07K 2319/73; C12N 2740/15043; C12Y 306/04013

(57) ABSTRACT

In various aspects and embodiments, the invention provides a method of controlling at least one cellular process in a mammalian cell via self-assembly of a synthetic organelle from expression of a scaffold protein capable of undergoing liquid-liquid phase separation (LLPS) tagged with a coiled coil. The method also comprises inserting one or more coiled coil or dimerization tags into the genome of a target mammalian cell; tags are operatively linked to at least one cellular decision making protein, thereby forming a sequesterable construct upon scaffold expression and controlling at least one cellular process.

### CROSS-REFERENCE TO RELATED APPLICATION

The present application is entitled to priority under 35 U.S.C. § 119 (e) to U.S. Provisional Patent Application No. 63/228,484, filed Aug. 2, 2021, which is hereby incorporated by reference in its entirety herein.

### STATEMENT REGARDING FEDERALLY SPONSORED RESEARCH OR DEVELOPMENT

This invention was made with government support under EB028320 awarded by the National Institutes of Health and 1720530 awarded by the National Science Foundation. The government has certain rights in the invention.

### SEQUENCE LISTING

The present application contains a Sequence Listing which has been submitted in XML format via Patent Center and is hereby incorporated by reference in its entirety. Said XML file, created on Aug. 2, 2022, is named “046483_7343WO1_Sequence Listing.xml” and is 32,339 bytes in size.

### BACKGROUND OF THE INVENTION

Cells can enhance the rate and fidelity of biochemical reactions through subcellular compartmentalization. For example, membrane-bound organelles, such as the nucleus and lysosome, display highly selective partitioning of biological cargo. Their restricted permeability increases reactivity via enforced proximity and ensures specificity by insulating components from competing reactions. Cells also contain membraneless organelle subcompartments, such as the nucleolus and P granules, that form through the self-assembly and coacervation of disordered proteins and RNA into mesoscale biomolecular condensates. By harnessing principles of protein self-assembly, it is possible to construct designer nano- or microcompartments inside a cell that encapsulate enzymes and substrates to control or augment their functions in living systems. There is a need in the art for new methods of interrogating cellular processes. This disclosure addresses that need.

### SUMMARY OF THE INVENTION

In one aspect, the disclosure provides a synthetic organelle comprising a first nucleic acid sequence encoding an intrinsically disordered protein (IDP) scaffold comprising three arginine/glycine-rich (RGG) domains, a first high-affinity coiled-coil (CC) tag, and a first promoter; and a second nucleic acid sequence encoding a client protein, a second CC tag, and a second promoter.

In another aspect, the disclosure provides a method of controlling at least one cellular process in a cell, the method comprising administering to the cell: a first nucleic acid sequence encoding an IDP scaffold comprising three RGG domains, a first high-affinity coiled-coil (CC) tag, and a first promoter; and a second nucleic acid sequence encoding a client protein, a second CC tag, and a second promoter, wherein the client protein is a cellular decision making protein, wherein when the scaffold is expressed, a sequesterable construct is formed and at least one cellular process is controlled.

In another aspect, the disclosure provides a method of controlling at least one cellular process in a mammalian cell, the method comprising administering to the cell: a first nucleic acid sequence encoding an IDP scaffold comprising three RGG domains, a first high-affinity coiled-coil (CC) tag, and a first promoter; and a second nucleic acid sequence encoding a client protein and a second CC tag, wherein the CC tag is inserted into the genome of the mammalian cell in a region encoding a cellular decision making protein, wherein when the scaffold is expressed, a sequesterable construct is formed and at least one cellular process is controlled.

In various embodiments of the above aspects or any other aspect of the invention delineated herein, the first or second CC tag is selected from the group consisting of SZI, SZ2, TsCC(A), and TsCC(B). In certain embodiments, the first CC tag is TsCC(A) and the second CC tag is TsCC(B). In certain embodiments, when TsCC(A) interacts with TsCC(B), the client protein is sequestered in the synthetic organelle, and wherein when temperature is raised, the client protein is released from the synthetic organelle. In certain embodiments, the CC tag is encoded by the nucleotide sequence of any of SEQ ID NOs: 7, 8, 9, or 10: or comprises the amino acid sequence of any of SEQ ID NOs: 17, 18, 19, or 20.

In certain embodiments, the RGG domains are RGG1, RGG2, and RGG3 from the Caenorhabditis elegans LAF-1 protein. In certain embodiments, the RGG domains are encoded by the nucleotide sequence of any of SEQ ID NOs: 1-6, or comprise the amino acid sequence of SEQ ID NO: 16.

In certain embodiments, the client protein is an endogenous enzyme. In certain embodiments, the client protein regulates a cellular function. In certain embodiments, the first and/or second nucleic acid encodes a photocleavable protein or a fluorescent tag.

In certain embodiments, the photocleavable protein or fluorescent tag is selected from the group consisting of PhoCl, PhoCl 2F, EGFP, mScarlet, iRFP and mCherry. In certain embodiments, when the synthetic organelle is exposed to light, the photocleavable protein is cleaved and the client is released. In certain embodiments, the photocleavable protein or fluorescent tag is encoded by a nucleotide sequence of any of SEQ ID NOs: 11, 12, or 13.

In certain embodiments, the first and/or second nucleic acid encodes a drug-induced dimerization domain. In certain embodiments, the drug-induced dimerization domain is FRB or FKBP. In certain embodiments, the drug-induced dimerization domain is encoded by the nucleotide sequence of any of SEQ ID NOs: 14 or 15: or comprises the amino acid sequence of any of SEQ ID NOs: 24 or 25.

In certain embodiments, the first promoter is an inducible promoter and the second promoter is a constitutive promoter. In certain embodiments, the second promoter is an endogenous promoter.

Another aspect of the disclosure provides a synthetic organelle comprising a first nucleic acid encoding an intrinsically disordered protein (IDP) scaffold comprising three arginine/glycine-rich (RGG) domains, a first high-affinity coiled-coil (CC) tag, and a first promoter; and a second nucleic acid encoding a client protein, a second CC tag, and a second promoter, wherein the second promoter is an endogenous promoter and the second CC tag tags an endogenous genomic loci.

Another aspect of the disclosure provides a cell comprising any of the synthetic organelles contemplated herein. In certain embodiments, the cell is a mammalian cell. In certain embodiments, the cell is a human cell.

Another aspect of the disclosure provides a lentiviral vector comprising any of the synthetic organelles contemplated herein.

Another aspect of the disclosure provides a lentiviral vector comprising a nucleotide sequence encoding a promoter, and an IDP scaffold comprising three RGG domains and a CC tag. In certain embodiments, the CC tag is Syn ZIPI or TsCC(A). In certain embodiments, the promoter is a constitutive CMV viral promoter or a Tet-ON-3G drug-inducible promoter. In certain embodiments, the promoter is a tetracycline responsive TRE3G promoter.

Another aspect of the disclosure provides a cell comprising any of the lentiviral vectors contemplated herein.

In certain embodiments, the cell further comprises a nucleic acid encoding a client protein, a second CC tag, and a second promoter. In certain embodiments, the cell further comprises a nucleic acid encoding artTA transactivator. In certain embodiments, the cell is a mammalian cell. In certain embodiments, the cell is a human cell. In certain embodiments, the cell further comprises a packaging plasmid and/or an envelope plasmid.

### BRIEF DESCRIPTION OF THE DRAWINGS

The following detailed description of preferred embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIGS. 1A-1G: Robust cargo recruitment to synthetic condensates via protein-protein interaction domains. FIG. 1A: Schematic overview of assembling synthetic organelles from disordered scaffold proteins to target clients and predictably modulate cellular functions: scaffold: of arginine/glycine-rich (RGG) domains, green fluorescent protein (GFP) tag and a high-affinity coiled-coil (CC) tag under the control of an inducible promoter: client: fluorophore (mScarlet) with a cognate CC under the control of a constitutive promoter. FIG. 1B: Client recruitment strategies included SYNZIPs, thermally responsive coiled coils and rapamycin-induced dimerization domains. FIG. 1C: Representative images of a yeast cells expressing a scaffold and client with cognate SYNZIP (left) or thermally responsive (right) CC pairs. Merged images show strong recruitment of client to condensates. FIG. 1D: Comparison of mean condensate number (top) and volume (bottom) for each scaffold type: n=60 cells for SYNZIP and n=75 cells for TsCC(A) scaffolds. Error bars represent s.d. Significance was calculated by unpaired, two-tailed t-test: ****P<0.0001. FIG. 1E: Steady-state cytoplasmic concentration of scaffold outside of condensates (C_{cyto}) as a function of total cellular concentration (C_{cell}) for 30 cells per scaffold type. The dashed line represents a slope of 1: cond, condensate. FIG. 1F: Violin plots of the fraction of total scaffold protein present in condensates for cells as in FIG. 1D. Significance was calculated by unpaired, two-tailed t-test: ****P<0.0001. FIG. 1G: Violin plots of the fraction of total client recruitment to condensates with each CC pair compared to expected percentage for condensates of the same size without recruitment in cells as in FIG. 1D. Significance was calculated by one-way analysis of variance (ANOVA): ****P<0.0001.

FIGS. 2A-2J: Control of cellular behavior through targeted insulation of native enzymes in synthetic organelles. FIG. 2A: Schematic illustration of the regulation of a cell cycle control system. FIG. 2B: The hypothesis was that insulation of Cdc24 within synthetic organelles will block signaling and cell polarization, resulting in no proliferation. FIG. 2C: Representative images of cells expressing TsCC(A)-(RGG)₃ scaffolds and tagged clients, showing native Cdc24-mScarlet-TsCC(B) and Cdc5-mScarlet-TsCC(B) enriched in synthetic condensates. FIG. 2D: Violin plots of the fraction of total client protein in synthetic condensates compared to the expected values for condensates with no recruitment for 50 cells per strain from 3-5 fields of view (FOVs). Significance was calculated by unpaired, m two-tailed t-test: ****P<0.001. FIG. 2E: Predictions and representative brightfield images of cell morphologies for tagged Cdc24 (top) and Cdc5 (bottom) before (left) and after (right) inducing the expression of scaffold to form condensates. FIG. 2F: Percentage of cells arrested (unbudded) in the indicated strains: n=150 cells per strain pooled from three independent trials. Error bars represent s.d. Significance was calculated by one-way ANOVA (NS, not significant: **P<0.01). FIG. 2G: Growth rate in liquid culture for indicated strains as in FIG. 2F averaged from three independent trials. Error bars represent s.d. Significance was calculated by linear regression (NS, not significant: ****P<0.0001). FIG. 2H: Violin plots of the fraction of total Bnr1-mScarlet-TsCC(B) in condensates compared to expected values for condensates with no recruitment: 50 cells from 305 FOVs. FIG. 2I: Representative images of phalloidin-stained Bnr1ADAD-mScarlet-TsCC(B) BNI1Δ cells with or without condensates show altered spatial organization of the actin cytoskeleton following Brnl sequestration. FIG. 2J: Distribution of average phalloidin fluorescence across the cell body for 50 cells per condition from five FOVs. Cells lose polarization. The inset is a diagram of cell orientation: AU, arbitrary units: L, left: R, right.

FIGS. 3A-3D: Control of cell proliferation by induced target sequestration. FIG. 3A: Schematic representation of rapamycin-inducible client recruitment and Cdc24-mScarlet-FKBP to FRB-(RGG): scaffold condensates. FIG. 3B: Representative images of cells expressing Cdc24-mScarlet-FKBP and FRB-scaffold before (top) and after (bottom) the addition of Rap. FIG. 3C: Quantification of the fraction of scaffold and client (Cdc24) in synthetic condensates over time as in FIG. 3B for 16 cells. The shaded area represents the 95% confidence interval. FIG. 3D: Spot dilution assays of the same yeast strain grown containing Cdc24-mScarlet-FKBP either lacking or expressing condensates with or without 100 nM Rap added to the medium.

FIGS. 4A-4H: Optical and thermal client release for reversible cell cycle control. FIG. 4A: Schematic representation of cargo release using thermally responsive coiled coils. FIG. 4B: Schematic prediction of the switch from cell arrest to proliferation following the release of Cdc24 from synthetic condensates. FIG. 4C: Average percentage of cells arrested over time. Scaffolds were expressed at 25° C. to form synthetic condensates and induce arrest. After 6 h, cells were heated to the indicated temperatures to induce client release. Cells with no condensate expression represent the baseline (light gray). Lines represent temperature increases to 37° C. and 42° C., for 1 and 2h (left) and overnight (right). Cells with scaffold expression maintained at 25° C. are represented in black. Data from three trials are derived from n=4,369 cells. Error bars represent s.d. FIG. 4D: Quantification of the fraction of Cdc24 in condensates before and after heating: n=10.cells. FIG. 4E: Schematic representation of light-induced release of Cdc24 from condensates via PhoCl photocleavage on the scaffold. FIG. 4F: Quantification of the loss of Cdc24-mScarlet-TsCC(B) signal from condensates after short pulses of illumination (10 s each): n=11 cells. FIG. 4G: Percentage of cells arrested over time after inducing synthetic condensate expression comparing no illumination to 10 min of ultraviolet (UV) light exposure at a 4-h time point: n=4,928 cells pooled from three trials. The shaded area represents the s.d. FIG. 4H: Cycling cells between arrest, proliferation and arrest: 25° C. from 0 to 6h, scaffold is expressed: 42° C. from 6 to 8 h, client is released and arrest is reversed: 25° C. from 8 to 12 h, client is rerecruited and arrest is reimposed. Data are averaged from three trials and a total of 4,988 cells. Error bars represent s.d.

FIGS. 5A-5E: CRISPR-tagged endogenous clients enrich within synthetic condensates expressed in mammalian cells. FIG. 5A: Representative images of Racl-mCherry-TsCC(B) localization in U2OS cells expressing (RGG); scaffold with the cognate (TsCC(A)) or non-matching (SZ1) coiled coil: scale bars, 10 μm. FIG. 5B: Violin plots of client enrichment for Racl-mCherry-TsCC(B) and ERK1-mCherry-TsCC(B) in the presence of synthetic condensates with matching and non-matching coiled coils: n=65 and 20 cells from four and three independent experiments, respectively. FIG. 5C: Percentage of scaffold and indicated endogenously tagged protein in condensates. For FIGS. 5B-5C, n=65 and 20 cells pooled from four and three independent experiments for Rac1 and ERK1, respectively. FIG. 5D: Representative images of Par6-mCherry-TsCC(B) localization at the plasma membrane with no scaffold expression (top) and with scaffold expression and condensate formation (bottom). FIG. 5E: Average of line scans of Par6 at the cell boundary for cells without scaffold expression, with non-matching scaffolds and with scaffolds with cognate coiled coils: n=10 cells for each line scan. Error bars represent s.d.

FIGS. 6A-6J: Properties of in vivo synthetic condensates. FIG. 6A: Temperature dependence of condensate assembly as a function of scaffold RGG domain valency. Representative images of yeast cells expressing galactose induced GFP tagged scaffold with 1, 2, or 3 RGG domains at different temperatures. FIG. 6B: Heat map: quantitation of turbidity data of purified proteins from Schuster et al., 2018. FIG. 6C: Heat map: number of condensates per cell as a function of temperature and RGG domain valency. FIG. 6D: Fluorescence recovery after photobleaching (FRAP) of condensates formed by (RGG)₃ scaffold: n=10 condensates. Shaded area, 95% CI. FIG. 6E: Fluorescence loss in photobleaching (FLIP) of condensates formed by (RGG)₃ scaffold. n=13 cells. FIG. 6F: Steady state cytoplasmic scaffold concentration outside of condensates (C_{cyto}) as a function of cellular concentration (C_{cell}) for 30 cells per scaffold type. Dashed line, slope of 1. FIG. 6G: Average enrichment of scaffold protein in condensates for SZ1-(RGG)₃ or TsCC(A)-(RGG)₃. n=164 and 97 condensates respectively. Error bars, s.d. FIG. 6H: Representative images of exogenously expressed mScarlet-SZ2 and mScarlet-TsCC(B) diffusely distributed in cytosol in the absence of condensates. FIG. 6I: Representative images of cells expressing FRB-(RGG)₃ scaffold and mScarlet-FKBP as a client. The client is diffuse in the cytosol before the addition of Rap and concentrated in condensates after Rap addition. FIG. 6J: Quantitation of the fraction of client protein as in i localized to condensates over time after Rap addition. n=15 cells. Shaded area 95% CI.

FIGS. 7A-7J: Condensate expression relocalizes tagged native clients and regulates cell growth. FIG. 7A: Representative images of tagged natively expressed Cdc24 show its cortical localization. FIG. 7B: Representative images of tagged natively expressed Cdc5 show its punctate localization to spindle pole body. FIG. 7C: Images of the same Cdc24-mScarlet-TsCC(B) cell before and after induced expression of TsCC(A)-(RGG); scaffold for 6 hr, show loss of cortical Cdc24 signal and partitioning to synthetic condensate. FIG. 7D: Client recruitment to condensates specifically depends on CC tag interaction: Cdc24 does not interact with (RGG)₃ condensates that lack the interaction tag. FIG. 7E: Left, scheme: cortical Cdc24 is relocalized from cortex to synthetic condensates after induction of scaffold. Right, Average cortical Cdc24-mScarlet-TsCC(B) signal before and after TsCC(A)-(RGG)₃ scaffold expression (6 h). n=20 cells before and after hours of galactose induction. Significance calculated by unpaired, two-tailed, t-test (****, p<0.0001). FIG. 7F: Kinetics of loss of cortical Cdc24-mCherry-TsCC(B) signal concomitant with cellular accumulation of expressed TsCC(A)-(RGG)₃ scaffold upon induction with galactose for 20 cells over 6 hours. Shaded area, s.d. FIG. 7G: Cell proliferation: measurements of cell density (OD600) over time for indicated Cdc24 strains in liquid media containing galactose. FIG. 7H: Growth assay for Cdc24 strains: five-fold serial dilution of indicated strains grown on solid-media containing glucose or galactose. FIG. 7I: Average cell area of mother cells only increases upon TsCC(A)-(RGG)₃ expression in Cdc24-mScarlet-TsCC(B) cells, consistent with cell cycle arrest in G1. FIG. 7J: Growth assay for Cdc5 strains: five-fold serial dilution of indicated strains grown on solid-media containing glucose or galactose. In all cases, growth defect depends on presence of tagged client and expression of scaffold to form condensates. Phenotype is not observed with only native client tagging or only scaffold expression.

FIG. 8A-8F: Reversible control of cell proliferation-arrest state. FIG. 8A: Representative images of Cdc24-mScarlet-TsCC(B) cells expressing TsCC(A)-(RGG)₃ scaffold at the indicated temperatures for 14 hours. Thermally responsive coiled-coil pair dissociate upon heating to 42° C., releasing client to promote cell polarity and reversing the cell cycle arrest associated with Cdc24 sequestration to condensates. FIG. 8B: Representative images of Cdc24-mScarlet-TsCC(B) in the presence of TsCC(A)-PhoC12f-(RGG); before and after illumination with 405 nm light. FIG. 8C: Schematic of client release strategy: Cdc24 is tagged with PhoCl-TsCC(B). 405 nm light results in PhoCl cleavage and client release. FIG. 8D: Percentage of cells expressing Cdc24-mScarlet-TsCC(B) arrested (unbudded cells) over time after scaffold induction+/−illumination. n=4048 cells in total pooled from three trials. FIG. 8E: Prediction: cycling of cell state between budded-arrested-budded-arrested. FIG. 8F: Representative images of cells at the indicated time points. Wildtype levels of budding at time 0 h. Cells incubated in galactose at 25° C. from 0)-6 h timepoints to induce condensate formation, blocking budding, then heated from 6 h to 8 h timepoints, promoting polarization and budding and cooled back to 25° C. and arrested by 12 h.

FIGS. 9A-9E: CRISPR tagging strategy for targeted client sequestration in mammalian cells. FIG. 9A: Schematic of CRISPR tagging approach to endogenous loci in mammalian cells and expression of the scaffold by a CMV promoter. FIG. 9B: PCR validation of CRISPR tagging in mammalian cell lines. Only tagged strains show a PCR product of the expected size as indicated. FIG. 9C: Representative images of tagged ERK1 robustly partitions to synthetic condensates formed by expression of TsCC(A)-(RGG)₃ scaffold. FIG. 9D: Representative images of tagged Par6 localized to the cell cortex in the absence of scaffold expression (left) and to condensate structures when scaffold is expressed (right). FIG. 9E: Quantitation of cortical Par6-mCherry-TsCC(B) in the absence and presence of condensates with cognate coiled coil. n=10 cells for each condition. Error bars, s.d. Significance calculated by unpaired, two-tailed t-test. (**, p<0.01).

FIGS. 10A-10B: Lentivirus delivery platform for generating synthetic condensates in mammalian cells. FIG. 10A: Lentiviral constructs with a number of parameters varied: (i) Promoter: Constitutive or inducible, (ii) coiled-coil types for targeting clients to synthetic condensates. FIG. 10B: Use of lentiviral transduction to generate a stable monoclonal U20S cell line, showing constitutive synthetic organelle expression for more than 1 month.

FIGS. 11A-11C: Inducible expression of synthetic condensates in human cell lines. FIG. 11A: Transient transfection of HEK293T cells with vectors containing TRE3G-TsCCA-RGRR and the CMV-rTTA plasmids. Condensates are not expressed in the basal state but assemble quickly upon addition of doxycycline. FIG. 11B: Quantitation of organelle formation over time upon drug addition. FIG. 11C: Stable lentiviral integration of TRE3G-TsCCA-RGRR and the CMV-rTTA into U20S cells. Organelles are not expressed in basal state and robustly form 12 hr after addition of doxycycline. Scalebar 10 μm.

FIG. 12: Synthetic organelle formation in human primary cells. Expression of TSCCA-RGG-GFP-RGG-RGG scaffold in human primary foreskin fibroblast cells after lentiviral transduction. Images of condensates 2 days after transduction.

FIGS. 13A-13C: Optogenetic controlled release of exogenous client from synthetic condensates in mammalian cells. FIG. 13A: Schematic of client release scheme, based on photocleavage of PhoCl-2f domain encoded between client (iRFP) and recruitment tag (TSCCB coiled coil). FIG. 13B: 405 nm laser illumination promotes client release from condensates without disrupting their structure. Transiently transfected U2OS expressing TsCCA-RGRR scaffold and TSCCA-PhoCL-2f-iRFP client. Over course of 10 light pulses in 20 minutes, iRFP-tagged client is released from condensates, but synthetic organelles are largely stable. FIG. 13C: Quantitation of photobleaching from non-cleavable client and PhoCl and light mediated client release. Photocleavage of PhoCl leads to robust client release and is not a result of photobleaching effects.

FIGS. 14A-14G: Tuning Cₛₐₜ for condensation using noncovalent RGG multimerization via coiled-coil pairs. FIG. 14A: Schematic of assembly of higher order RGG disordered polypeptides by genetic fusion to cognate pairs of helical coiled coils. In OFF (monomer) state, monomer concentrations should be below their Cₛₐₜ and in ON (dimer) state, higher polymer valency (length) lowers the Cₛₐₜ such that the dimer condenses into liquid-like droplets. FIGS. 14B, 14D, 14F: Representative fluorescence microscopy images of condensates formed using 4 μM RGG monomer concentrations, 150 mM salt, pH 8.5. FIG. 14B: images of SZ1-RGG, RGG-SZ2, and mixture of SZ1-RGG and RGG-SZ2. FIG. 14C: Turbidity measurements at A600 for 6 μM of the control RGG-RGG dimer, indicated monomers or mixtures of monomers, over a range of temperatures. FIG. 14D: Images of P3-RGG, RGG-P4, and combination of P3-RGG and RGG-P4. FIG. 14E: Turbidity measurement using 6 μM of the indicated control, monomer and mixed monomer over a range of temperatures. FIG. 14F: Representative images of RGG-P5, RGG-P6, and mixture of RGG-P5 and RGG-P5. FIG. 14G: Turbidity measurement using 6 μM of the indicated control dimer, monomer and mixed monomer over a range of temperatures.

FIGS. 15A-15H: Temporal control of IDR multimerization and phase separation in vitro. FIG. 15A: Schematic representation for chemogenic dimerization of RGG polypeptides to form mesoscale liquid-like protein condensates. FIG. 15B: Representative images of liquid droplet formation through increased domain valency upon addition of dimerizer, Rapamycin, (Rap). Recombinant RGG-FKBP and RGG-FRB proteins, in the absence of Rap, do not form condensates in a buffer containing 10 μM protein and 0.2 μM tracer (RGG-GFP-RGG). Scale bar, 10 μm. Addition of Rap to the reaction rapidly induces dimerization, causing condensate formation similar to the RGG-RGG constitutive dimer. FIGS. 15C-15D: Quantitation of the kinetics of droplet formation upon equimolar addition of Rap. Average of three independent trials. Shaded area, StDev. FIG. 15E: Kinetics of solution clouding after addition of dimerizer in spectrophotometric turbidity assays: average of three experiments area shown. FIG. 15F: Phase transition temperature measured by turbidity assay shows induced dimerization of RGG-FKBP and RGG-FRB with Rap shifts the cloud point to higher temperatures, similar to constitutive RGG-RGG dimer: average of three experiments. FIG. 15G: Representative images from photobleaching and recovery of condensates composed of Rap mediated RGG-FKBP/RGG-FRB dimers marked by 0.2 μM RGG-GFP-RGG tracer. Scale bar, 5 μm. FIG. 15H: Quantification of FRAP indicating similar recovery kinetics of Rap induced vs. constitutive RGG-RGG dimers. n=30 condensates from two independent trials.

FIGS. 16A-16D: Optochemical regulation of IDR condensation. FIG. 16A: Chemical structure of photocaged rapamycin, dRap. FIG. 16B: Schematic of approach: RGG domains fused to FKBP or FRB tags do not dimerize in the presence of dRap. Upon illumination, dRap is uncaged to Rap, resulting in dimerization of RGG-FKBP and RGG-FRB. FIG. 16C: Pre- and post-illumination images of water-in-oil emulsions, stabilized by Cithrol DPHS surfactant, encapsulating 10 μM each of RGG-FKBP and RGG-FRB. 0.2 μM of RGG-GFP-RGG as a fluorescent tracer, and 5 μM dRap. Dotted line is the emulsion boundary. Prior to illumination, GFP signal is diffuse indicating no condensation. After 30 sec of illumination and uncaging of Rap, RGG condensates appear, indicating dRap uncaging and protein dimerization. Scale bar, 10 μm. FIG. 16D: Kinetics measured from images of optically induced droplet formation inside emulsions, as in C. n=10 emulsions. Shaded area, StDev.

FIGS. 17A-17E: Leveraging induced dimerization to trigger synthetic condensate formation in living cells. FIG. 17A: Scheme for encoding and expression of RGG polypeptides in cells, sensitive to small molecule induced dimerization and condensation. RGG-GFP-FKBP and RGG-FRB constructs are integrated into the yeast genome controlled by an inducible GAL1 promotor. FIG. 17B: Representative images for strains described in A, showing addition of 20 μM Rap triggers condensate formation within minutes in live cells. FIG. 17C: Average number of droplets formed per yeast cell in the (n=105 cells). Shaded area, 95% CI. FIG. 17D: Optical regulation of condensation in cells following illumination to photo-uncage dRap. FIG. 17E: Average number of droplets formed per cell following 10 sec of 405 nm laser illumination (n=66 cells). Shaded area, 95% CI.

FIG. 18A-18C: Selective cargo recruitment to condensates via chemogenic dimerization. FIG. 18A: Schematic of RGG-RGG condensates, which cannot selectively recruit FRB-tagged client protein (mCherry-FRB). Client does not enrich in condensates because it cannot interact with free RGG polypeptides. Right, representative fluorescent images of condensates from 10 μM RGG-RGG marked with 0.2 μM RGG-GFP-RGG tracer, and 5 μM of client mCherry-FRB. Client is excluded from RGG-RGG condensates. FIG. 18B: Scheme for cargo recruitment via rapamycin through dimerization with RGG-FKBP which partitions to condensed phase. Right, representative images of condensates from 10 μM each of RGG-FKBP and RGG-FRB, in the presence of 10 μM Rap and 0.2 μM RGG-GFP-RGG tracer. Addition of 5 μM mCherry-FRB (client) results in robust and selective enrichment to condensed phase. FIG. 18C: Kinetics of client enrichment after addition of mCherry-FRB as in A and B from three independent experiments. Shaded area, 95% CI.

FIGS. 19A-19E. FIG. 19A: 4 μM of constitutively dimerized RGG-RGG condensates with 0.2 μM RGG-GFP-RGG as a fluorescent tracer. FIG. 19B: Comparison of condensate formation of P3-RGG, RGG-P4, and their mixture at increasing protein concentrations. FIG. 19C: Protein condensation of RGG constructs with varying location (N- or C-terminus) of P3 or P4 tags, alone and in combination at 4 μM and 6 μM concentrations. In all cases 0.2 μM RGG-GFP-RGG serves as a fluorescent tracer. FIG. 19D: Combination of 4 μM RGG constructs with non-cognate coiled coils shows no condensate formation. FIG. 19E) Lowering pH to 6.8 results in monomeric 4 μM RGG-P5 and RGG-P6 condensation alone as well as in combination. 0.2 μM RGG-GFP-RGG serves as a fluorescent tracer.

FIGS. 20A-20B. FIG. 20A: Representative images of FRAP experiments in which entire droplets of 10 μM FKBP and FRB tagged RGG domains with 0.2 μM RGG-GFP-RGG tracer were photobleached and recovery was tracked. FIG. 20B: Quantitation of fluorescence recovery from photobleaching whole droplets, comparing RGG-RGG vs. Rap induced RGG-FKBP-FRB-RGG dimer. N=30 droplets, pooled from two independent trials. Shaded area, StDev.

FIGS. 21A-21C. FIG. 21A: Quantitation of the kinetics of condensate growth and expansion over time in yeast, following Rap induction as in FIG. 17B. Shaded area, 95% CI. FIG. 21B: Quantitation of condensate growth following light uncaging of dRap to trigger multimerization, as in FIG. 17D. Shaded rea, 95% CI. FIG. 21C: Comparison of kinetics of chemogenic vs. optical induction of condensates in cells. Average values are shown.

FIG. 22: Schematic of augmenting cells with synthetic organelles that function as fast-acting protein switches. Platform has potential to be run in two functional modes: insulator, or bioreactor.

FIG. 23: Biomolecular condensates as membraneless organelles in the cell. They form from self-assembly of disordered proteins and RNA into mesoscale subcompartments.

FIG. 24: Disordered RGG domain from P granule protein Laf-1 is necessary and sufficient for condensate formation in vitro.

FIG. 25: Summary of sequence determinants of Laf-1 RGG LLPS.

FIG. 26: Mutation of Laf-1 RGG to raise or lower its critical concentration for LLPS does not alter the liquid-like nature of condensates in vitro.

FIG. 27: Demonstration of utility of Laf-1 RGG to form engineered coacervates in vitro.

FIG. 28: Embedding of optical regulatory handles into Laf-1 RGG sequence to control its LLPS using light.

FIG. 29: Schematic summary of applications of synthetic organelle platform to sequester pathway components in key subcellular processes, including cell cycle control system, cell death via apoptosis and cell fate control.

FIG. 30: Schematic summary of application of synthetic organelle as a hub to rewire signal transmission.

### DETAILED DESCRIPTION OF THE INVENTION

### A. Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting. As used herein, the singular form “a,” “an,” and “the” include plural references unless the context clearly dictates otherwise.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods. Unless otherwise clear from context, all numerical values provided herein are modified by the term about.

As used in the specification and claims, the terms “comprises,” “comprising,” “containing,” “having,” and the like can have the meaning ascribed to them in U.S. patent law and can mean “includes,” “including,” and the like.

As used herein, the term “cellular decision making protein” means a protein or peptide that when present and able to interact with the cell, produces an observable, phenotypic change in the cell relative to the absence of the protein. A person of ordinary skill will recognize that when a cellular decision making protein is sequestered and unable to interact with other factors it is functionally equivalent to the absence of the cellular decision making protein. By way of non-limiting example, Cdc24 is a cellular decision making protein that influences the cell cycle in yeast.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame. When a heterologous nucleic acid is operably linked to a non-regulatory sequence the term refers to a relationship between the two nucleic acids such that when one is transcribed the other is transcribed in the intended reading frame and that the relationship with local regulatory sequences is the same such that the two nucleic acids express a single peptide.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Designer Membraneless Organelles that Sequester Native Factors for Control of Cell Behavior

Synthetic condensates or membraneless organelles can be assembled in a cell from the expression of disordered protein sequences above their saturation concentration. Low-complexity sequences from Fus and other FET family members, resilin-like sequences and arginine/glycine-rich (RGG) domains from Laf-1 have been used to generate synthetic condensates in bacterial, yeast and mammalian systems. The utility of a disordered protein platform for generating condensates in vitro in synthetic cell-like compartments has also been demonstrated. The 168-amino acid disordered RGG domain of the Caenorhabditis elegans P granule protein LAF-1 is necessary and sufficient for phase separation and does not require RNA for self-assembly. Importantly, the valency of the RGG domain tunes the critical concentration for liquid-liquid phase separation (LLPS), and real-time reduction of valency promotes condensate disassembly. Further, enzymatic and optical release of a solubilization domain from RGG initiates condensate assembly. In addition, transient expression in cells leads to the formation of liquid-like micron-size condensates.

In living cells, biomolecular condensates and membraneless organelles sequester client enzymes or RNAs to either increase enzymatic flux or to insulate them from other cellular machinery. For example, in response to various stresses, mammalian cells form stress granules to sequester proteins, RNA and elongation factors, a response that prevents stress-induced cellular senescence. Herein, a synthetic membraneless organelle platform was developed that functions to sequester and insulate native enzymes for modular control over cellular functions. These designer organelles have broad utility in cell biology and engineering applications, exhibiting restricted permeability, showing highly selective and efficient enrichment of specific cargoes and being capable of controllable client release. Throughout this disclosure, the platform described is referred to as ‘synthetic organelles’ or ‘condensates’, which are used interchangeably herein.

Enforced localization of exogenously expressed clients in cells has been demonstrated using synthetic condensate systems. A common strategy tags the exogenous client with the same disordered protein sequence domain present on the intrinsically disordered protein (IDP) scaffold to direct partitioning to synthetic condensates. However, concerns arise about integrating large, disordered domains into endogenous gene loci, particularly whether they are orthogonal or may alter endogenous protein functionality. Further, it is not clear whether this IDP-tagging approach is generalizable and capable of sequestering a majority of the endogenously expressed target protein in the cell. Therefore, this disclosure provides a substantial advance in the development of a synthetic condensate platform in which a majority of the scaffold protein partitions to the condensate to achieve high fractional client recruitment. Combined with a modular strategy for localizing clients without disrupting their native function, for example, using coiled-coil interaction motifs, a key capability is functional insulation of native enzymes. An additional engineering demand is reversibility of client recruitment, enabling controlled release from a designer organelle to restore pathway function and switch cells between functional states.

In the present disclosure, a synthetic membraneless organelle system was developed to insulate and functionally knockdown essential native enzymes via compartmentalization and achieve modular control of cellular behavior. Successful engineering of a number of platform functions was demonstrated: nearly full partitioning of scaffold and native clients to the synthetic organelle was achieved by screening through IDP valencies and recruitment tags. By genomic tagging of native gene loci, functional insulation of enzymes that regulate the cell cycle control system and actin cytoskeleton was shown, thereby switching cells from a proliferation state to an arrested state and from polarized to isotropic cytoskeletal organization. The feasibility of rapid induced client recruitment and switching of cell behavior was demonstrated. Further, thermal and optical strategies for controlled release of clients localized to the synthetic organelle for reversible control of the cell activity state were demonstrated. Additionally, the feasibility of implementing this platform in mammalian cells by CRISPR tagging of endogenous gene loci to efficiently partition and relocalize native enzymes was demonstrated. This designer membraneless organelle system, embedded with interaction tags, offers a powerful and generalizable chemical biology tool for controlling cellular activities. The applications of this approach range from real-time probing of pathways in cell biology to mesoscale protein switches for cellular engineering and synthetic biology.

In one aspect, the invention includes a synthetic organelle comprising a first nucleic acid sequence encoding an intrinsically disordered protein (IDP) scaffold comprising three arginine/glycine-rich (RGG) domains, a first high-affinity coiled-coil (CC) tag, and a first promoter; and a second nucleic acid sequence encoding a client protein, a second CC tag, and a second promoter.

In certain embodiments, the first or second CC tag is selected from the group consisting of SZ1, SZ2, TsCC(A), and TsCC(B). In certain embodiments, the first CC tag is TsCC(A) and the second CC tag is TsCC(B). In certain embodiments, when TsCC(A) interacts with TsCC(B), the client protein is sequestered in the synthetic organelle, and wherein when temperature is raised, the client protein is released from the synthetic organelle. In certain embodiments, the CC tag is encoded by the nucleotide sequence of any of SEQ ID NOs: 7, 8, 9, or 10: or comprises the amino acid sequence of any of SEQ ID NOs: 17, 18, 19, or 20.

In certain embodiments, the RGG domains are RGG1, RGG2, and RGG3 from the Caenorhabditis elegans LAF-1 protein. In certain embodiments, the RGG domains are encoded by the nucleotide sequence of any of SEQ ID NOs: 1-6, or comprise the amino acid sequence of SEQ ID NO: 16.

In certain embodiments, the client protein is an endogenous enzyme. In certain embodiments, the client protein regulates a cellular function. In certain embodiments, the first and/or second nucleic acid encodes a photocleavable protein or a fluorescent tag.

In certain embodiments, the photocleavable protein or fluorescent tag is selected from the group consisting of PhoCl, PhoCl 2F, EGFP, mScarlet, iRFP and mCherry. In certain embodiments, when the synthetic organelle is exposed to light, the photocleavable protein is cleaved and the client is released. In certain embodiments, the photocleavable protein or fluorescent tag is encoded by a nucleotide sequence of any of SEQ ID NOs: 11, 12, or 13.

In certain embodiments, the first and/or second nucleic acid encodes a drug-induced dimerization domain. In certain embodiments, the drug-induced dimerization domain is FRB or FKBP. In certain embodiments, the drug-induced dimerization domain is encoded by the nucleotide sequence of any of SEQ ID NOs: 14 or 15: or comprises the amino acid sequence of any of SEQ ID NOs: 24 or 25.

In certain embodiments, the first promoter is an inducible promoter and the second promoter is a constitutive promoter. In certain embodiments, the second promoter is an endogenous promoter.

Another aspect of the invention includes a synthetic organelle comprising a first nucleic acid encoding an intrinsically disordered protein (IDP) scaffold comprising three arginine/glycine-rich (RGG) domains, a first high-affinity coiled-coil (CC) tag, and a first promoter; and a second nucleic acid encoding a client protein, a second CC tag, and a second promoter, wherein the second promoter is an endogenous promoter and the second CC tag tags an endogenous genomic loci.

Another aspect of the invention includes a cell comprising any of the synthetic organelles contemplated herein. In certain embodiments, the cell is a mammalian cell. In certain embodiments, the cell is a human cell.

Another aspect of the invention includes a lentiviral vector comprising any of the synthetic organelles contemplated herein.

Another aspect of the invention includes a lentiviral vector comprising a nucleotide sequence encoding a promoter, and an IDP scaffold comprising three RGG domains and a CC tag. In certain embodiments, the CC tag is Syn ZIPI or TsCC(A). In certain embodiments, the promoter is a constitutive CMV viral promoter or a Tet-ON-3G drug-inducible promoter. In certain embodiments, the promoter is a tetracycline responsive TRE3G promoter.

Another aspect of the invention includes a cell comprising any of the lentiviral vectors contemplated herein.

In certain embodiments, the cell further comprises a nucleic acid encoding a client protein, a second CC tag, and a second promoter. In certain embodiments, the cell further comprises a nucleic acid encoding a rtTA transactivator. In certain embodiments, the cell is a mammalian cell. In certain embodiments, the cell is a human cell. In certain embodiments, the cell further comprises a packaging plasmid and/or an envelope plasmid.

In another aspect, the invention includes a method of controlling at least one cellular process in a mammalian cell via self-assembly of a synthetic organelle from expression of a scaffold protein capable of undergoing liquid-liquid phase separation (LLPS) tagged with a coiled coil. The method also comprises inserting one or more coiled coil or dimerization tags into the genome of a target mammalian cell: tags are operatively linked to at least one cellular decision making protein, thereby forming a sequesterable construct upon scaffold expression and controlling at least one cellular process.

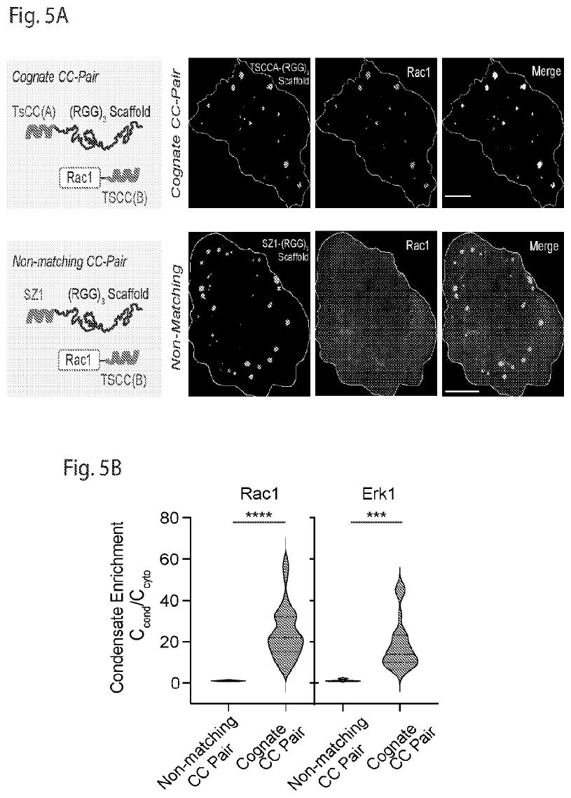

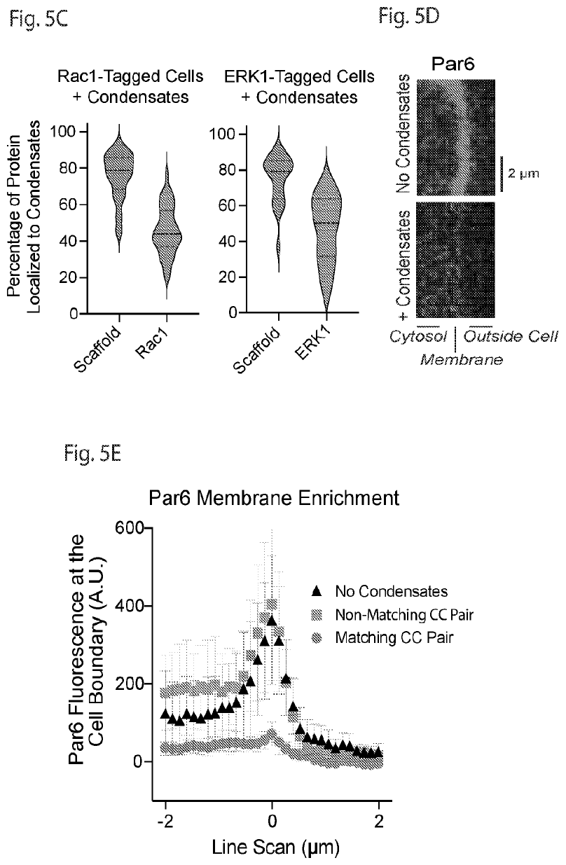

Without meaning to be limited by theory, the present disclosure presents evidence that shows that methods of sequestering proteins in synthetic organelles are effective in mammalian cells, for example in FIGS. 5A-5E and accompanying discussion. In various embodiments the mammalian cell is a human cell. In various embodiments, the target cell may be any cell in which the skilled person wishes to control a cellular process. In various embodiments, the cellular process is the cell cycle.

In another aspect, the disclosure provides a method of controlling at least one cellular process in a cell, the method comprising administering to the cell: a first nucleic acid sequence encoding an IDP scaffold comprising three RGG domains, a first high-affinity coiled-coil (CC) tag, and a first promoter; and a second nucleic acid sequence encoding a client protein, a second CC tag, and a second promoter, wherein the client protein is a cellular decision making protein, wherein when the scaffold is expressed, a sequesterable construct is formed and at least one cellular process is controlled. In another aspect, the disclosure provides a method of controlling at least one cellular process in a mammalian cell, the method comprising administering to the cell: a first nucleic acid sequence encoding an IDP scaffold comprising three RGG domains, a first high-affinity coiled-coil (CC) tag, and a first promoter; and a second nucleic acid sequence encoding a client protein and a second CC tag, wherein the CC tag is inserted into the genome of the mammalian cell in a region encoding a cellular decision making protein, wherein when the scaffold is expressed, a sequesterable construct is formed and at least one cellular process is controlled.

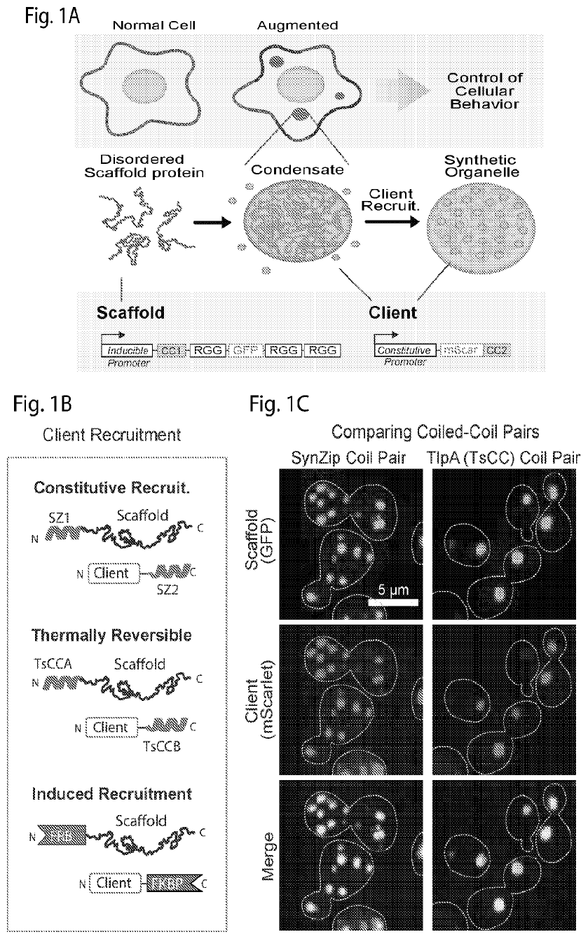

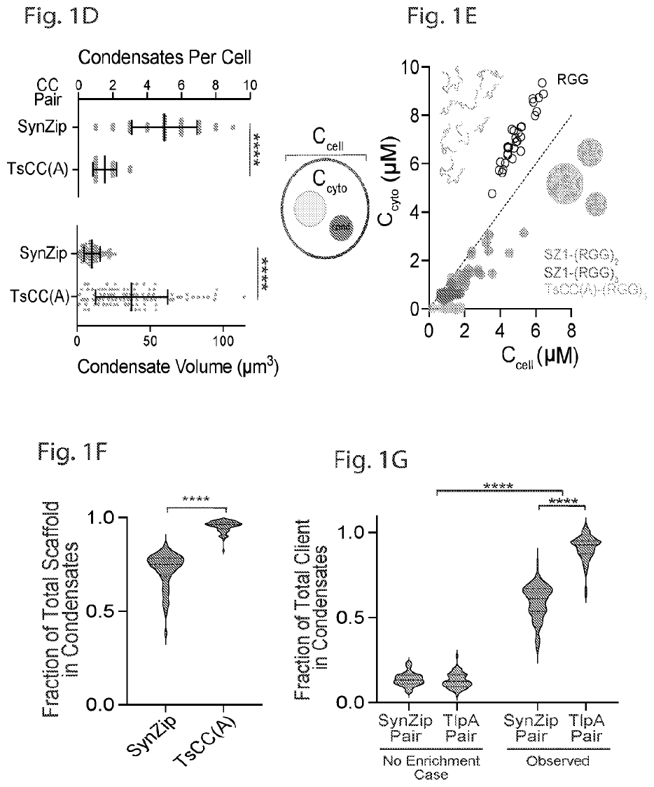

The term sequesterable construct refers to a tagged cellular decision making protein, wherein the construct becomes sequestered in a synthetic membraneless organelle or if the tag is inducible will become sequestered upon receiving a stimulus. Without meaning to be limited by theory, when the cellular decision making protein is sequestered, the cell will display the phenotype typically observed when the cellular decision making protein is absent. The sequesterable construct may be a single polypeptide or may include multiple peptides assembled by non-covalent interactions. By way of non-limiting example, the sequesterable construct may be any of the constructs, scaffolds, or clients, depicted in FIG. 1A or FIG. 1B.

In various embodiments tags may be inserted into the genome of a target cell by any means known in the art and a skilled person is able to select an appropriate technique. In various embodiments, the tag is inserted into the genome of the target cell using CRISPR. In various embodiments, in which the method is applied to an exogenous protein, the exogenous protein is inserted simultaneously with the tag or tags. In various embodiments, the tags comprise one or more selected from the group consisting of Syn ZIPI, SZI, SZ2, TsCC(A), and TsCC(B), PhoCl, PhoCl 2F, EGFP, mScarlet, iRFP and mCherry

In various embodiments, the tags are inducible. In this context, inducible tags may be induced to sequester or release a protein by exposing the cell containing the construct to a stimulus. By way of nonlimiting example, the stimulus may initiate cleavage of the construct or may change the tertiary structure of the tag. The release or sequestration may or may not be reversible. Again, by way of non-limiting example cleavage of the tag will irreversibly release the cellular decision making protein (client protein) from the synthetic membraneless organelle. In contrast, in various embodiments, a reversible tag allows the cellular decision making protein to be released or sequestered upon the application and removal of a certain stimulus.

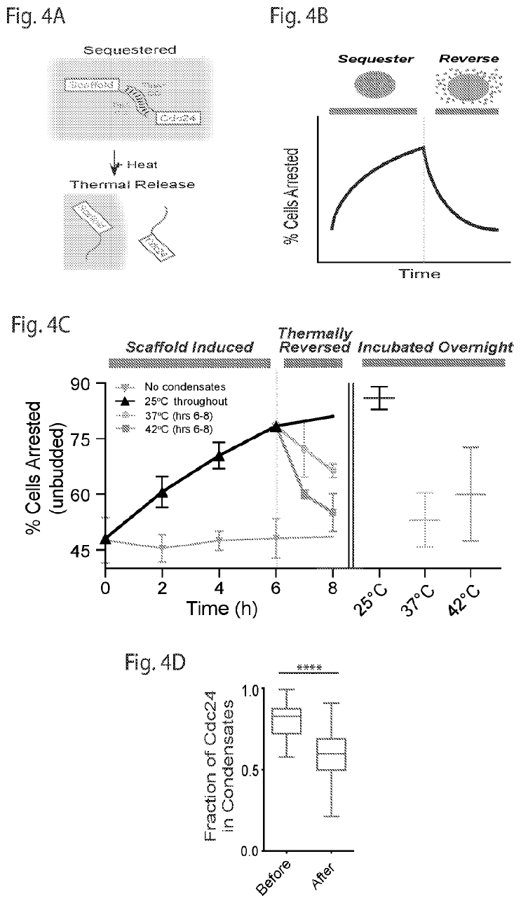

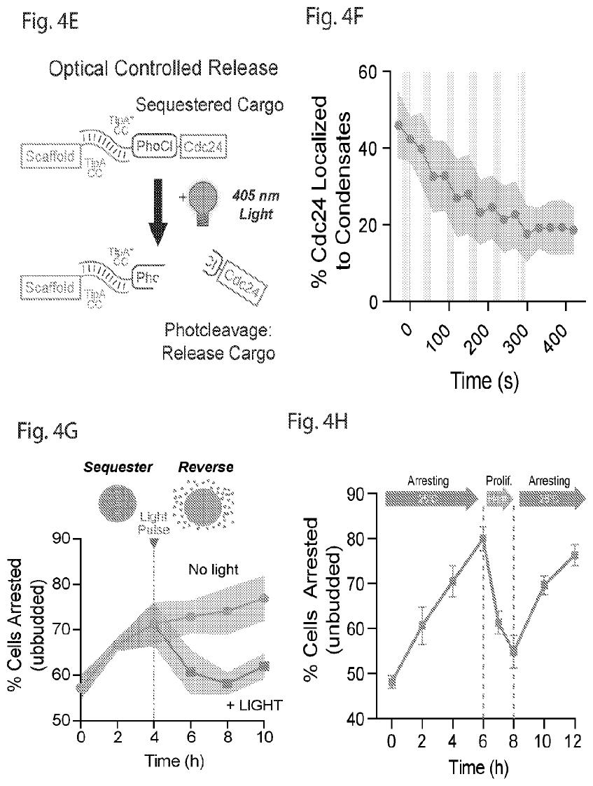

In various embodiments, the one or more tags are thermally or optically reversible. A person of skill in the art will appreciate that optically inducible here means that the stimulus is based on light or temperature. In various embodiments, the tags comprise TsCC(A) and/or TsCC(B). These are thermally induced LLPS tags that sequester the sequesterable construct below a threshold temperature and release it when above a threshold temperature as illustrated in FIGS. 4A-4D. In various embodiments, the optically reversible sequesterable construct includes an optically cleavable linker that is cleaved upon exposure to a certain wavelength of light. Upon cleavage, the cellular decision making protein is released. In various embodiments, the tag comprises PhoCI which is optically cleavable as depicted in FIGS. 4E-4H.

Accordingly, in various embodiments, the method further comprises applying a stimulus to the mammalian cell, thereby sequestering or releasing the sequesterable construct and controlling the outcome of the at least one cellular process. In various embodiments, the stimulus comprises at least one selected from the group consisting of exposure to light and a change in temperature.

C. Sequences used in the study:
Laf-1 RGG Sequences (Disordered Domains):
Yeast RGG1 (SEQ ID NO: 1)
GAATCGAATCAATCTAACAACGGTGGATCGGGAAACGCAGCATTGAATCGTG
GGGGCCGTTACGTACCACCACACTTGAGAGGCGGAGATGGTGGTGCCGCCGC
TGCTGCCTCAGCCGGAGGCGACGACAGACGCGGTGGGGCCGGGGGTGGTggct
atCGTAGAGGTGGTGGAAATTCAGGGGGCGGCGGCGGAGGAGGCTACGATCG
TGGATACAACGACAACCGGGATGACCGTGACAATCGGGGCGGCTCCGGCGGT
TATGGCCGGGACAGAAATTACGAGGATCGCGGTTATAACGGGGGTGGTGGGG
GAGGAGGCAATCGCGGCTACAATAATAACCGCGGTGGGGGTGGAGGCGGCT
ATAATCGGCAGGACCGTGGAGACGGCGGTTCTAGTAATTTCTCGCGGGGAGG
GTATAACAACAGAGATGAGGGCTCAGACAACAGAGGCTCAGGCAGATCATA
CAACAACGATCGGCGCGACAATGGTGGTGACGGG
Yeast RGG2 (SEQ ID NO: 2)
GAATCCAATCAATCCAATAACGGTGGCTCGGGTAATGCCGCTCTGAATCGTG
GTGGCCGCTATGTGCCGCCGCATCTGCGTGGTGGCGATGGTGGTGCAGCAGC
AGCTGCATCTGCTGGCGGTGATGACCGTCGCGGCGGTGCGGGCGGTGGCGGT
TATCGTCGCGGCGGTGGCAATAGTGGTGGCGGTGGCGGTGGCGGTTATGATC
GTGGTTACAATGACAACCGTGATGACCGCGATAACCGTGGCGGTTCCGGCGG
TTACGGTCGTGATCGTAATTATGAAGACCGTGGTTACAACGGCGGTGGCGGT
GGCGGTGGCAATCGTGGCTATAACAATAACCGTGGTGGCGGTGGCGGTGGCT
ACAATCGCCAGGATCGTGGTGACGGTGGCAGCTCTAACTTTTCGCGCGGTGG
CTATAATAACCGTGATGAAGGCAGCGACAACCGCGGTAGTGGCCGTTCCTAC
AATAACGATCGTCGCGACAATGGTGGCGATGGC
Yeast RGG2 (SEQ ID NO: 3)
GAATCTAACCAATCAAACAATGGCGGCTCCGGTAACGCGGCGCTTAACAGGG
GCGGTCGTTACGTGCCTCCGCACCTGAGAGGGGGCGACGGAGGTGCGGCTGC
TGCCGCGAGTGCCGGTGGTGATGACAGGCGTGGTGGTGCGGGTGGCGGGGGC
TATCGTCGTGGAGGCGGIAATTCCGGGGGAGGTGGTGGTGGGGGTTACGACA
GAGGGTACAATGATAACCGTGATGATCGTGACAACAGAGGCGGGAGTGGCG
GTTACGGTAGGGATCGTAACTATGAAGACAGAGGCTATAACGGGGGTGGTGG
TGGCGGTGGCAATAGAGGTTATAATAACAACCGTGGCGGCGGTGGAGGCGGG
TATAACAGACAGGATAGAGGGGACGGCGGCAGTAGCAACTTCTCCCGTGGCG
GATATAACAACCGTGACGAAGGCTCTGACAATAGGGGTTCTGGTAGGTCTTA
CAATAATGATAGGAGAGACAATGGCGGCGACggt
Human RGG1 (SEQ ID NO: 4)
GAATCGAATCAATCTAACAACGGTGGATCGGGAAACGCAGCATTGAATCGTG
GGGGCCGTTACGTACCACCACACTTGAGAGGCGGAGATGGTGGTGCCGCCGC
TGCTGCCTCAGCCGGAGGCGACGACAGACGCGGTGGGGCCGGGGGTGGTGGC
TATCGTAGAGGTGGTGGAAATTCAGGGGGCGGCGGCGGAGGAGGCTACGATC
GTGGATACAACGACAACCGGGATGACCGTGACAATCGGGGCGGCTCCGGCGG
TTATGGCCGGGACAGAAATTACGAGGATCGCGGTTATAACGGGGGTGGTGGG
GGAGGAGGCAATCGCGGCTACAATAATAACCGCGGTGGGGGTGGAGGCGGC
TATAATCGGCAGGACCGTGGAGACGGCGGTTCTAGTAATTTCTCGCGGGGAG
GGTATAACAACAGAGATGAGGGCTCAGACAACAGAGGCTCAGGCAGATCAT
ACAACAACGATCGGCGCGACAATGGTGGTGACGGG
Human RGG2 (SEQ ID NO: 5)
GAATCCAATCAGTCCAATAATGGGGGCAGCGGGAACGCGGCCTTGAATCGCG
GTGGGCGGTACGTTCCTCCTCATTTAcGAGGTGGAGATGGAGGCGCTGCAGCC
GCtGCaAGTGCAGGaGGTGAcGAtCGTaGAGGcGGaGCTGGAGGcGGaGGtTATAG
aCGGGGAGGGGGAACTCAGGAGGcGGcGGtGGtGGaGGgTAcGACAGAGGTTAT
AACGACAATCGAGATGATCGGGATAACCGAGGTGGGAGCGGGGGTTACGGG
CGCGATCGCAATTACGAGGACAGGGGTTATAATGGTGGGGGCGGAGGAGGC
GGAAACCGGGGGTATAACAATAATCGGGGAGGGGGAGGTGGAGGGTATAAC
CGCCAGGACCGAGGTGACGGAGGGTCAAGCAATTTTTCACGCGGGGGCTACA
ACAACCGCGACGAGGGCAGTGACAATCGGGGTAGTGGCAGGAGTTACAACA
ATGATAGAAGGGACAACGGCGGTGATGGG
Human RGG2 (SEQ ID NO: 6)
GAGTCTAATCAGAGCAATAACGGGGGTAGTGGCAATGCCGCGCTCAATCGGG
GAGGCCGCTACGTGCCTCCTCACCTCAGAGGGGGCGATGGGGGCGCCGCTGC
CGCTGCGAGCGCCGGTGGCGATGATCGGCGGGGAGGAGCGGGTGGAGGTGG
TTATCGCCGAGGTGGCGGGAACAGTGGGGGTGGAGGAGGCGGTGGATATGAT
CGAGGTTACAACGACAATCGGGACGATCGAGATAACAGAGGGGGTTCAGGT
GGATACGGAAGAGACAGGAATTACGAGGACAGAGGATACAATGGCGGAGGC
GGTGGGGGAGGTAATAGAGGATACAACAATAATCGAGGAGGCGGGGGGGGA
GGGTACAATCGCCAAGACCGCGGTGATGGAGGAAGTTCTAACTTTAGCAGAG
GTGGATACAACAATAGGGATGAAGGCTCAGACAACAGAGGTTCTGGTCGAAG
CTACAATAACGACAGACGCGACAACGGTGGCGATGGG
Coiled-Coil Pairs Sequences:
SZ1 (SEQ ID NO: 7)
Atgaacttggtagcccagcttgagaacgaagttgcgagtctggaaaacgaaaacgagacgttgaagaaaaagaacttgcacaa
gaaagatttgattgcctacttggaaaaggaaattgccaatctgcgtaaaaagattgaggaa
SZ2 (SEQ ID NO: 8)
ATGgcgcgtaacgcatatcttcgtaagaaaattgcacggctgaaaaaggacaatctacagttggaacgcgacgagcagaacc
tcgagaagatcattgctaatctgcgcgacgagattgctcgcttagaaaacgaagtcgcatcacatgagcag
TsCC(A) (SEQ ID NO: 9)
gtggaagtggctgaagaagtgaaggccgtctccgccgcgctgtccgaacgcatcacccagctggcgacagaactgaatgaca
aggcggtccgggctgcagaacgccgggttgcggaagtcacgcgtgctgccggtgaacagaccgcacaggcagagcgggag
ctggccgacgccgcgcagacagtcgacgacctggaagaaaaactggTtgaactgcaggacagatatgacagtttgacgctgg
cgctggagtcagaacgttcactgcgtcagcagcatgatgtggagatggcccagctgaaagagcgtcttgcggccgctgaagag
aatacccgtcagcgacgtgaacggtatcaggagcagaagacagtgctgcaggatgcgcttaatgcggagcaggcacagcaca
aaaacacgcgggaagacctgcagaaacgactggagcaaatttctgTcgaagctaatgcgcgtacagaagaactgaagtctga
acgcgataaagtcaatactTtccttacccgccttgaatcgcaggaaaatgcgctggcctcacgtcgtcagcagcatctggccacc
cgcgaaacgctgcagcaacgcctcgagcaggccatcgctgacacgcaggcgcgcgccggtgagattgcacttgaacgtgac
agagtcagcagcctcaccgcaaggctggaatcgcaggaaaaggcctcctcggagcaactggtgcgtatgggcagtgaaatag
ccagtctgacagagcgttgcacacagctggaaaaccagcgtgatgatgcccgtctggagacgatgggggagaaagaaacggt
cgcggcactgcgtggtgaggctgaagccctgaagcgtcagaaccagtcactgatggcggcgctttcaggcaataaacagacC
GGTGGCCAGAATGCG
TsCC(B) (SEQ ID NO: 10)
gtggaagtggctgaagaagtgaaggccgtctccgccgcgctgtccgaacgcatcacccagctggcgacagaactgaatgaca
aggcggtccgggctgcagaacgccgggttgcggaagtcacgcgtgctgccggtgaacagaccgcacaggcagagcgggag
ctggccgacgccgcgcagacagtcgacgacctggaagaaaaactggTtgaactgcaggacagatatgacagtttgacgctgg
cgctggagtcagaacgttcactgcgtcagcagcatgatgtggagatggcccagctgaaagagcgtcttgcggccgctgaagag
aatacccgtcaggaagaggaacggtatcaggagcagaagacagtgctgcaggatgcgcttaatgcggagcaggcacagcac
aaaaacacgcgggaagacctgcagaaacgactggagcaaatttctgTcgaagctaatgcgcgtacagaagaactgaagtctg
aacgcgataaagtcaatactTtccttacccgccttgaatcgcaggaaaatgcgctggcctcagaagaacagcagcatctggcca
cccgcgaaacgctgcagcaacgcctcgagcaggccatcgctgacacgcaggcgcgcgccggtgagattgcacttgaacgtg
acagagtcagcagcctcaccgcaaggctggaatcgcaggaaaaggcctcctcggagcaactggtgcgtatgggcagtgaaat
agccagtctgacagagcgttgcacacagctggaaaaccagcgtgatgatgcccgtctggagacgatgggggagaaagaaac
ggtcgcggcactgcgtggtgaggctgaagccctgaagcgtcagaaccagtcactgatggcggcgctttcaggcaataaacag
ACCGGTGGCCAGAATGCG
Photocleavable Protein and Fluorescent Tag Sequences:
Phocl 2F (SEQ ID NO: 11)
gtgatccctgactacttcaagcagagcttccccgagggctacagctgggagcgcagcatgacctacgaggacggcggcatctg
catcgccaccaacgacatcacaatggagggggacagcttcatcaacaagatccacttccagggcacgaacttcccccccaacg
gccccgtgatgcagaagaggaccgtgggctgggaggccagcaccgagaagatgtacgagcgcgacggcgtgctgaagggc
gacgtgaagatgaagctgctgctgaagggcggcggccactatcgctgcgactaccgcaccacctacaaggtcaagcagaagc
ccgtaaagctgcccgactgccacttcgtggaccaccgcatcgagatcctgagccacgacaaggactacaacaaggtgaagctg
tacgagcacgccgtggccaagacttccaccgacagcatggacgagctgtacaagggtggcagcggtggcatggtgagcaag
ggcgaggagaccattacaagcgtgatcaagcctgacatgaagaacaagctgcgcatggagggcaacgtgaacggccacgcc
ttcgtgatcgagggcgagggcagcggcaagcccttcgagggctctcagacgattgatttggaggtgaaggagggcgccccgc
tgcccttcgcctacgacatcctgaccaccgccttccactacggcaaccgcgtgttcaccaagtacccacgg
EGFP (SEQ ID NO: 12)
atggtgagcaagggcgaggagctgttcaccggggtggtgcccatcctggtcgagctggacggcgacgtaaacggccacaagt
tcagcgtgtccggcgagggcgagggcgatgccacctacggcaagctgaccctgaagttcatctgcaccaccggcaagctgcc
cgtgccctggcccaccctcgtgaccaccctgacctacggcgtgcagtgcttcagccgatatccagatcatatgaagcagcacga
tttctttaaaagtgcgatgcccgagggttatgttcaggagcgcaccatcttcttcaaggacgacgggaattataagacccgcgctg
aggttaagtttgaaggggatacccttgttaacagaattgagctgaagggtatagactttaaagaagatggcaatatattggggcac
aagctcgaatataattacaacagtcataacgtatatatcatggctgacaagcaaaagaatggcataaaagtgaattttaagatacga
cacaatatagaggatggtagcgtccagcttgcggatcattaccagcagaatacaccaattggtgacggcccagtactgctgccg
gataatcactatctcagtacccagagtgcattgtccaaagacccgaacgagaaacgcgatcacatggtactgctggaatttgtaac
ggctgctggaattacattgggaatggacgagttgtacaag
mCherry (SEQ ID NO: 13)
atggtgagcaagggcgaggaggataacatggccatcatcaaggagttcatgcgcttcaaggtgcacatggagggctccgtgaa
cggccacgagttcgagatcgagggcgagggcgagggccgcccctacgagggcacccagaccgccaagctgaaggtgacc
aagggtggccccctgcccttcgcctgggacatcctgtcccctcagttcatgtacggctccaaggcctacgtgaagcaccccgcc
gacatccccgactacttgaagctgtccttccccgagggcttcaagtgggagcgcgtgatgaacttcgaggacggcggcgtggtg
accgtgacccaggactcctccctgcaggacggcgagttcatctacaaggtgaagctgcgcggcaccaacttcccctccgacgg
ccccgtaatgcagaagaagaccatgggctgggaggcctcctccgagcggatgtaccccgaggacggcgccctgaagggcga
gatcaagcagaggctgaagctgaaggacggcggccactacgacgctgaggtcaagaccacctacaaggccaagaagcccgt
gcagctgcccggcgcctacaacgtcaacatcaagttggacatcacctcccacaacgaggactacaccatcgtggaacagtacg
aacgcgccgagggccgccactccaccggcggcatggacgagctgtacaag
Drug-Induced Dimerization Domain Sequences:
FKBP (SEQ ID NO: 14)
ATGAAAGGCGTGCAGGTGGAGACTATCTCCCCAGGAGACGGGCGCACCTTCC
CCAAGCGCGGCCAGACCTGCGTGGTGCACTACACCGGGATGCTTGAAGATGG
AAAGAAATTTGATTCCTCCCGGGACAGAAACAAGCCCTTTAAGTTTATGCTAG
GCAAGCAGGAGGTGATCCGAGGCTGGGAAGAAGGGGTTGCCCAGATGAGTG
TGGGTCAGAGAGCCAAACTGACTATATCTCCAGATTATGCCTATGGTGCCACT
GGGCACCCAGGCATCATCCCACCACATGCCACTCTCGTCTTCGATGTGGAGCT
TCTAAAACTGGAA
FRB (SEQ ID NO: 15)
GAGATGTGGCATGAAGGCCTGGAAGAGGCATCTCGTTTGTACTTTGGGGAAA
GGAACGTGAAAGGCATGTTTGAGGTGCTGGAGCCCTTGCATGCTATGATGGA
ACGGGGCCCCCAGACTCTGAAGGAAACATCCTTTAATCAGGCCTATGGTCGA
GATTTAATGGAGGCCCAAGAGTGGTGCAGGAAGTACATGAAATCAGGGAATG
TCAAGGACCTCACCCAAGCCTGGGACCTCTATTATCATGTGTTCCGACGAATC
TCAAAGCAG
Protein Sequences:
Laf-1 RGG (SEQ ID NO: 16)
ESNQSNNGGSGNAALNRGGRYVPPHLRGGDGGAAAAASAGGDDRRGGAGGGG
YRRGGGNSGGGGGGGYDRGYNDNRDDRDNRGGSGGYGRDRNYEDRGYNGGG
GGGGNRGYNNNRGGGGGGYNRQDRGDGGSSNFSRGGYNNRDEGSDNRGSGRS
YNNDRRDNGGDG
SZ1 (SEQ ID NO: 17)
MNLVAQLENEVASLENENETLKKKNLHKKDLIAYLEKEIANLRKKIEE
SZ2 (SEQ ID NO: 18)
MARNAYLRKKIARLKKDNLQLERDEQNLEKIIANLRDEIARLENEVASHEQ
TsCC(A) (SEQ ID NO: 19)
VEVAEEVKAVSAALSERITQLATELNDKAVRAAERRVAEVTRAAGEQTAQAERE
LADAAQTVDDLEEKLVELQDRYDSLTLALESERSLRQQHDVEMAQLKERLAAA
EENTRQRRERYQEQKTVLQDALNAEQAQHKNTREDLQKRLEQISVEANARTEEL
KSERDKVNTFLTRLESQENALASRRQQHLATRETLQQRLEQAIADTQARAGEIAL
ERDRVSSLTARLESQEKASSEQLVRMGSEIASLTERCTQLENQRDDARLETMGEK
ETVAALRGEAEALKRQNQSLMAALSGNKQTGGQNA
TsCC(B) (SEQ ID NO: 20)
VEVAEEVKAVSAALSERITQLATELNDKAVRAAERRVAEVTRAAGEQTAQAERE
LADAAQTVDDLEEKLVELQDRYDSLTLALESERSLRQQHDVEMAQLKERLAAA
EENTRQEEERYQEQKTVLQDALNAEQAQHKNTREDLQKRLEQISVEANARTEEL
KSERDKVNTFLTRLESQENALASEEQQHLATRETLQQRLEQAIADTQARAGEIAL
ERDRVSSLTARLESQEKASSEQLVRMGSEIASLTERCTQLENQRDDARLETMGEK
ETVAALRGEAEALKRQNQSLMAALSGNKQTGGQNA
Phocl 2F (SEQ ID NO: 21)
VIPDYFKQSFPEGYSWERSMTYEDGGICIATNDITMEGDSFINKIHFQGTNFPPNGP
VMQKRTVGWEASTEKMYERDGVLKGDVKMKLLLKGGGHYRCDYRTTYKVKQ
KPVKLPDCHFVDHRIEILSHDKDYNKVKLYEHAVAKTSTDSMDELYKGGSGGM
VSKGEETITSVIKPDMKNKLRMEGNVNGHAFVIEGEGSGKPFEGSQTIDLEVKEG
APLPFAYDILTTAFHYGNRVFTKYPR
EGFP (SEQ ID NO: 22)
MVSKGEELFTGVVPILVELDGDVNGHKFSVSGEGEGDATYGKLTLKFICTTGKLP
VPWPTLVTTLTYGVQCFSRYPDHMKQHDFFKSAMPEGYVQERTIFFKDDGNYKT
RAEVKFEGDTLVNRIELKGIDFKEDGNILGHKLEYNYNSHNVYIMADKQKNGIK
VNFKIRHNIEDGSVQLADHYQQNTPIGDGPVLLPDNHYLSTQSALSKDPNEKRDH
MVLLEFVTAAGITLGMDELYK
mCherry (SEQ ID NO: 23)
MVSKGEEDNMAIIKEFMRFKVHMEGSVNGHEFEIEGEGEGRPYEGTQTAKLKVT
KGGPLPFAWDILSPQFMYGSKAYVKHPADIPDYLKLSFPEGFKWERVMNFEDGG
VVTVTQDSSLQDGEFIYKVKLRGTNFPSDGPVMQKKTMGWEASSERMYPEDGA
LKGEIKQRLKLKDGGHYDAEVKTTYKAKKPVQLPGAYNVNIKLDITSHNEDYTI
VEQYERAEGRHSTGGMDELYK
FKBP (SEQ ID NO: 24)
MKGVQVETISPGDGRTFPKRGQTCVVHYTGMLEDGKKFDSSRDRNKPFKFMLG
KQEVIRGWEEGVAQMSVGQRAKLTISPDYAYGATGHPGIIPPHATLVFDVELLKL
E
FRB (SEQ ID NO: 25)
EMWHEGLEEASRLYFGERNVKGMFEVLEPLHAMMERGPQTLKETSFNQAYGRD
LMEAQEWCRKYMKSGNVKDLTQAWDLYYHVFRRISKQ

### D. Protein Condensate Formation Via Controlled Multimerization of Intrinsically Disordered Sequences

Proteins harboring low complexity or intrinsically disordered sequences (IDRs) are capable of undergoing liquid-liquid phase separation to form mesoscale condensates that function as biochemical niches with the ability to concentrate or sequester macromolecules and regulate cellular activity. Engineered disordered proteins are used to generate programmable synthetic membraneless organelles in cells. Phase separation is governed by the strength of interactions among polypeptides, with multivalency enhancing phase separation at lower concentrations. Enzymatic control of IDR valency from multivalent precursors was demonstrated to dissolve condensed phases. Herein, noncovalent strategies were developed to multimerize an individual IDR, the RGG domain of LAF-1, using protein interaction domains to regulate condensate formation in vitro and in living cells. First, modular dimerization of RGG domains at either terminus were characterized using cognate high-affinity coiled coil pairs to form stable condensates in vitro. Second, temporal control was demonstrated over phase separation of RGG domains fused to FRB and FKBP in the presence of dimerizer. Further, using a photocaged dimerizer, optically induced condensation was achieved both in cell-sized emulsions and within cells. Collectively, these modular tools allowed multiple strategies to promote phase separation of a common core IDR for tunable control of condensate assembly.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Designer Membraneless Organelles Sequester Native Factors for Control of Cell Behavior

### Materials & Methods

### Molecular Biology.

All plasmids were constructed using InFusion cloning (Takara Bio) and were verified by DNA sequencing. Yeast plasmids expressing RGG domain scaffolds were encoded in the integrating Yiplac211 (URA3, ampicillin resistance (AmpR) plasmid backbone downstream of the inducible GAL1 promoter. GALI, interaction tag, RGG and GFP sequences were generated by PCR and were cloned into the plasmid backbone between the XbaI and AgeI restriction sites. Plasmids expressing exogenous client (mScarlet) fused to a C-terminal interaction domain were encoded in the integrating Yiplac128 (LEU2, AmpR) plasmid backbone downstream of a constitutive MET17 promoter. PCR products encoding the MET17 promoter sequence, mScarlet and an interaction tag were cloned into Yiplac128 between the XbaI and AgeI cut sites. To generate PCR products for yeast knock-ins, fluorophore and interaction domain sequences were cloned into pfa6a::KANMX6 or pfa6a::HIS3 plasmid backbones. PCR products of TsCC(B), mScarlet-TsCC(B) and mScarlet-FKBP were generated from the previously cloned Yiplac 128 plasmids above and were cloned into the pfaba vectors using the PacI and AscI restriction sites. To generate a plasmid to knock-in PhoCl-TsCC(B), PCR products of the PhoCl and TsCC(B) sequences were cloned into pfa6a::KANMX6 between PacI and AscI restriction sites via InFusion ligation. For mammalian cell work, plasmids encoding scaffolds with interaction domains were cloned into pcDNA vectors downstream of a cytomegalovirus (CMV) promoter. GFP and RGG domains were cloned from gene fragments codon optimized for human expression (Integrated DNA Technologies). Sequences were cloned into the pcDNA backbone sequentially between the BamHI and XbaI restriction sites. For mammalian CRISPR knock-ins, Cas9 plasmids with the appropriate guide RNA (gRNA) and donor plasmids encoding a fluorophore and coiled-coil interaction domains were generated. To construct Cas9 plasmids, the pCas9-guide (OriGene Technologies) was used as a backbone, and a 20-nucleotide sequence encoding the gRNA targeting the C-terminal end of the gene of interest was assembled using duplexed oligos and was cloned between BamHI and BsmBI restriction sites according to the manufacturer's instructions. Donor plasmids were constructed using the pUC19 donor backbone (Takara) and encoded 600-1,000-base pair (bp) homology arms along with mCherry-TsCC(B) and a nourseothricin N-acetyl transferase resistance (NATR) cassette in between the homology arms. The mCherry-TsCC(B) sequence was first cloned into a pcDNA backbone using the BamHI and XbaI cut sites. A 1,000-bp 5′ homology arm was generated by PCR from synthesized gene fragments (Integrated DNA Technologies) and was cloned upstream of the mCherry sequence using the NheI and BamHI restriction sites. The NATR cassette and a 600-800-bp 3′ homology arm were then amplified and fused by two-step PCR. These 5′ and 3′ sequences were then PCR amplified and cloned into pUC19 between the HindIII and SacI restriction sites. In each case, the PAM site, located in one of the homology arms, was changed to prevent persistent cleavage by Cas9.

### Yeast Procedures.

Standard methodologies were followed for all experiments involving Saccharomyces cerevisiae. In all cases, the scaffold was under the control of the galactose inducible GAL1 promotor and was incorporated into the yeast URA3 locus using an integrating vector (Yiplac211) cut with EcoRV and standard lithium acetate transformation. Exogenously expressed clients under the control of the MET17 promotor were similarly integrated into the LEU2 locus with an integrating vector (Yiplac 128) after EcoRV digestion. To tag native genomic loci, PCR products of tags and drug resistance cassettes containing 40 to 50 bp of homology on either end of the C terminus of the target gene were transformed into yeast cells by lithium acetate/PEG transformation as previously described. BNI1 was deleted by replacing the open reading frame (ORF) with a TRP1 marker. The DAD domain of Bnr1 was internally deleted by Cas9-mediated gene editing by cotransforming yeast strains with a plasmid expressing Cas9 and a gRNA targeting the DAD domain of BNR1 and an 80-nucleotide oligo with homology to sequences upstream and downstream of the DAD domain.

For scaffold induction, yeast cells were first grown to saturation overnight in liquid YPD medium in a 25° C. shaking incubator. Cells were then washed three times in sterile water, diluted in YP+2% raffinose and incubated in a 25° C. shaking incubator for 6 to 8 h. Finally, yeast cells were diluted to an optical density at 600 nm (OD600) of 0.3 in YP+2% galactose, and induction proceeded overnight in the same shaking incubator or for hours on a microscope slide to track scaffold induction and cargo recruitment in the same cells. The final OD600 values of cultures used for experiments were between 0.4 and 0.8 except for the strain harboring TsCC(A)-scaffold and endogenous Cdc24 or Cdc5 tagged with mScarlet-TsCC(B) as their growth arrests with scaffold induction. For client partitioning studies in the presence of scaffold, cells were incubated overnight in galactose for ˜14 h.

For thermal reversal experiments, yeast cells harboring TsCC(A)-scaffold and Cdc24-TsCC(B) were grown in YPD medium as above and were washed and transferred to YP+2% raffinose overnight. Cells were then transferred into YP+2% galactose to trigger scaffold induction. Thermal reversal was performed after 6 h of scaffold expression by transferring 1 ml of each cell culture to a heated water bath for 1 and 2 h. For overnight thermal reversion, cells were maintained at 37° C. or 42° C. Samples were taken at the indicated time points, and cells were fixed with 4% paraformaldehyde (PFA) for 10 min (Ricca Chemical Company), centrifuged and washed three times with 1 ml of PBS and stored at 4° C. until imaging. For light-induced client release, yeast cells harboring a combination of TsCC(A)-PhoCl2f-scaffold and Cdc24-mScarlet-TsCC(B) or TsCC(A)-scaffold and Cdc24-PhoCl-TsCC(B) were grown, and scaffolds were expressed by switching to galactose, as above. After 4 h of scaffold expression, cells expressing TsCC(A)-PhoC12f-scaffold and Cdc24-mScarlet-TsCC(B) were imaged and exposed to 10-s pulses of 405-nm light on an Olympus IX81 inverted confocal microscope (Olympus Life Science) with a Yokogawa CSU-XI spinning disk, mercury lamp, 488- and 561-nm lasers and an iXon3 EMCCD camera (Andor) controlled by MetaMorph software (Molecular Devices). Cells expressing TsCC(A)-scaffold and Cdc24-PhoCl-TsCC(B) were induced as above, heated to 37° C., exposed to a 10-min pulse of UV light and then allowed to continue to grow in YP+2% galactose medium. Samples were taken at the indicated time points and fixed/stored as above until imaging. For phalloidin staining, 1 ml of cell culture was fixed with 4% PFA for 1 h and centrifuged and washed three times in 1 ml of PBS. Fixed cells were resuspended in 49 μl of PBS plus 1 μl of AlexaFluor568-phalloidin (Invitrogen) and rotated in the dark at room temperature overnight. Before imaging, cells were centrifuged and washed twice with PBS.

### Mammalian Cell Procedures.

U2OS human osteosarcoma cells were cultured in Eagle's minimal essential medium (EMEM: Quality Biological) supplemented with 10% fetal bovine serum (Gibco), 2 mM l-glutamine (Gibco) and 10 U ml-1 penicillin-streptomycin (Gibco) and were maintained at 37° C. in a humidified atmosphere with 5% CO2. Cells were split in a 1:3 ratio every 3 d, had been passaged for less than 2 months and were negative for known infection. Experiments were done with a confirmed viability of >95%, as determined by trypan blue staining (Gibco). For drug selection, cells were cultured in EMEM supplemented with 10% fetal bovine serum, 2 mM l-glutamine and 0.75 mg ml-1 G418 sulfate (MediaTech).

A CRISPR knock-in strategy was implemented to tag Rac1 and ERK1 at their native genomic loci. pUC19 donor plasmids (Takara Bio) were cloned harboring mCherry-TsCC(B) and a neomycin resistance cassette along with 600- to 1,000-bp homology arms as described above. Donor plasmids were cotransfected with Cas9 plasmids (OriGene Technologies) cloned with the two to three distinct gRNAs to target the gene of interest. Cotransfection of donor plasmids and pCas-gRNA plasmids was performed using Lipofectamine 2000 (Invitrogen) according to the manufacturer's protocol. Briefly, cells were seeded at 70% confluency in six-well flat-bottom tissue culture plates (CELLTREAT) 24 h before the transfection. On the day of transfection, 1,500 ng of donor plasmids and 500 ng of each pCas-gRNA plasmid were mixed in Opti-MEM reduced serum medium (Gibco). Lipofectamine 2000 was added at a 1:5 DNA-to-reagent ratio and incubated for 15 min before adding to the cells dropwise. Twenty-four hours after transfection, cells were trypsinzed and moved to 100-mm cell culture dishes (ThermoFisher Scientific). Cells were selected with drug for 7 d. After selection, cells were rested in medium without drug for 24 h and sorted based on mCherry expression using a BD FACSAria III cell sorter (BD Bioscience) with help from the flow cytometry core at the University of Pennsylvania. Briefly, cells were resuspended at 10×106 cells per ml in medium supplemented with 25 mM HEPES (Gibco). Before sorting, 1 μl of 1 μg ml-1 DAPI (Invitrogen) was added to the sample for live/dead staining. Cells were sorted into medium-expression and high-expression bins and were maintained for 2 weeks in complete medium until confluent. mCherry-positive cells were confirmed by fluorescence microscopy. CRISPR knock-in of tags to endogenous loci was confirmed via PCR.

For scaffold expression, postselection cells were seeded on 24-well glass-bottom plates (Greiner Bio-One) at 70% confluency. Twenty-four hours later, cells were transfected with 1,000 ng of GFP-tagged scaffold cloned into a pcDNA vector using X-tremeGENE 9 DNA transfection reagent (Sigma-Aldrich) at a 1:3 DNA-to-reagent ratio according to the manufacturer's protocol. In all cases, cells imaged were first tested for mycoplasma using a MycoAlert mycoplasma detection kit (Lonza) according to the manufacturer's protocols. All of the cells reported in this study were determined to be mycoplasma free.

### Microscopy.

Fluorescence microscopy imaging of yeast and mammalian cells was performed on an Olympus IX81 inverted confocal microscope (Olympus Life Science) equipped with a Yokogawa CSU-XI spinning disk, mercury lamp, 488- and 561-nm laser launches and an iXon3 EMCCD camera (Andor). Multidimensional acquisition was controlled by MetaMorph software (Molecular Devices). Samples were illuminated using a 488-nm laser and/or a 561-nm laser and were imaged through a ×100, 1.4-NA oil immersion objective. Z stacks were collected at a sampling depth appropriate for three-dimensional reconstitution. Brightfield transmitted light images used to assess yeast cell morphologies were also captured on a Nikon Eclipse Ti-U confocal microscope (Nikon) equipped with a Yokogawa CSU-XI spinning disk and a Photometrics Evolve Delta EMCCD camera (Teledyne Photometrics).

To image mesoscale condensates, budding yeast in YP medium containing 2% galactose were immobilized to glass coverslips treated with concanavalin A (ConA). For chemogenic induction of client recruitment, yeast cells in the same medium were first allowed to adhere to glass coverslips coated with ConA, and, subsequently, Rap was added to a final concentration of 20 μM.

For yeast photobleaching experiments (fluorescence recovery after photobleaching (FRAP) and fluorescence loss in photobleaching (FLIP)), a Roper iLas2 photoactivation system controlling a 405-nm laser was used. For FRAP, individual condensates were selected and photobleached, and fluorescence recovery in the bleached region was analyzed in ImageJ. For FLIP, half of a cell body was photobleached, and fluorescence loss from the condensate on the opposite half of the cell was analyzed in ImageJ. Mammalian U2OS cells were imaged 40 h after transient transfection with the after adhering to a 24-well glass-bottom plate (Greiner.Bio-One). In all cases, z stacks were collected to visualize the scaffold at the 488-nm wavelength and the client at a 561-nm wavelength using a ×100, 1.4-NA oil immersion objective.

### Image Analysis.

Analysis of condensates and clients in cells was performed in ImageJ. To quantify in vivo phase plots and determine Cₛₐₜ, cells expressing scaffold were imaged alongside wells containing purified GFP fusion proteins to generate a standard curve for fluorescence. C_{cyto} was calculated from the average background-subtracted fluorescence intensity of cytoplasmic signal and was converted to concentration using the calibration curve. To quantify scaffold and client recruitment to synthetic condensates in yeast and U2OS cells, we segmented cells and condensates using ImageJ. Objects were masked in the z plane of the image stack containing the largest portion of cells. Because U2OS cells are adherent and spread, masks were generated in the 488-nm channel by automatic thresholding using the MaxEntropy algorithm in ImageJ, and the lower boundary was manually set to be threefold higher than the average cytosolic signal. The particle analysis function in ImageJ was used to segment condensates larger than a five-pixel area. Background-subtracted measurements of 488-nm and 561-nm pixel intensity for masks for the condensates and cells were used to calculate an enrichment index (background-corrected fluorescence intensitycondensates/intensitycytosol). To estimate the fraction of scaffold or client partitioned to the organelles, the background-subtracted integrated pixel intensity for condensate mask areas was divided by the background-subtracted integrated pixel intensity of the cell mask (Σintensity condensates/Σintensity cell). Par6 signal at the cell cortex was analyzed by line scans in ImageJ using a line 30 pixels in length and thickened by 10 pixels. Line scans were then averaged, and background intensity was subtracted.

Quantification of cellular phenotypes (for example, budding indices and cell size) was performed in ImageJ using brightfield images of live cells or of fixed cells from time course experiments. Multiple FOVs were captured per experiment, and budding indices were generated by counting the fraction of cells that had a daughter cell (bud). Cell size measurements were performed by manual tracing of the outline of the mother cell to determine cell area. Distribution of AlexaFluor561-phalloidin staining was quantified by drawing a box that encompassed the entire cell body along the long axis of the cell and by plotting summed intensity as a function of position. Box position was determined by the position of the bud or location of polarized signal to the end of the mother cell. The longest cell axis was used in cases where a polarity axis could not be determined, such as in cells with sequestered Bnr1. Datasets were normalized to average mother cell fluorescence in cells that lacked condensates. The fluorescence profiles for at least 50 individual cells from each strain were rescaled by defining the back of the mother cell as 0 and the tip of polarized signal in G1 cells, or tip of the bud in other cell cycle stages, as 1.

### Statistics and Reproducibility.

Experiments were reproducible. All statistical analyses were performed in GraphPad Prism 9. To test the significance of two categories, an unpaired two-tailed t-test was used. To test significance of more than two categories, a one-way ANOVA was used. To compare differences in growth curves, significance was determined by linear regression analysis. In all cases, NS indicates not significant: *P≤0.05, **P≤0.01, ***P<0.001 and ****P<0.0001.

### Results

### Targeting Clients to Synthetic Membraneless Organelles

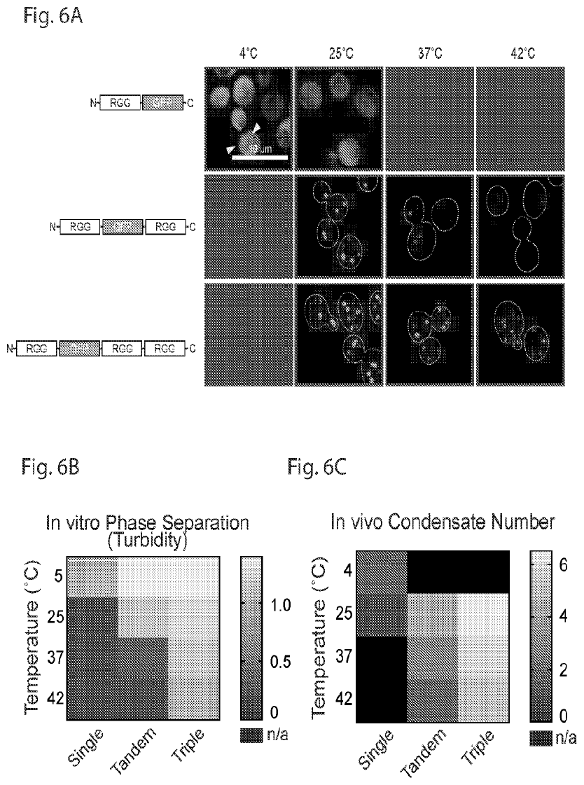

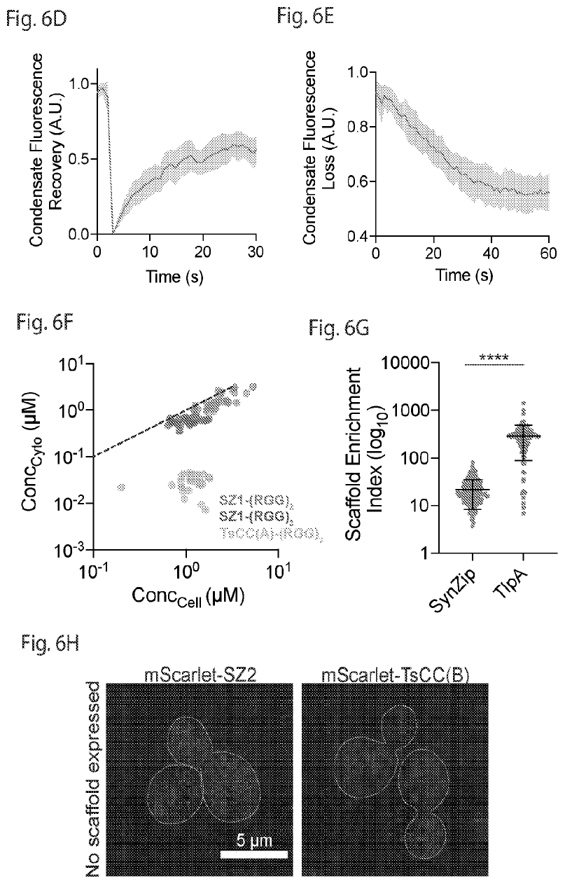

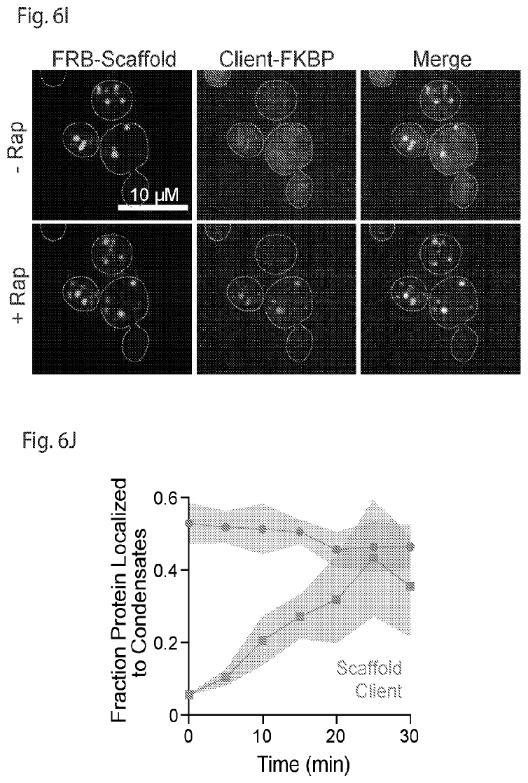

The first goal of the study was to augment living cells with synthetic compartments, screening them for temperature stability and critical concentration to achieve a high fraction of intrinsically disordered protein (IDP) scaffold in condensates. Constructs containing a single RGG domain have poor LLPS activity in vivo, consistent with previous in vitro findings (FIGS. 6A-6C). Addition of a second RGG domain allowed condensate formation at 25° C., but was not stable at higher temperatures (FIGS. 6A-6C). A scaffold encoding three RGG domains, however, allowed for robust condensate formation and stability over a wide range of temperatures (FIGS. 6A-6C). Importantly, these in vivo structures maintained liquid-like features (FIGS. 6D and 6E).

The second goal was to test various protein interaction motifs for tagging clients to stably or reversibly enforce their proximity to the synthetic organelle (FIG. 1A). Cognate interaction motifs were encoded on the N terminus of the IDP scaffold protein and C terminus of client proteins. The testing set included (1) short coiled-coil SYNZIP pairs SZI (SEQ ID NOs: 7 & 17) and SZ2 (SEQ ID NOs: 8 & 18), (2) thermally reversible coiled-coil domains, TsCC(A) (SEQ ID NOs: 9 & 19) and TsCC(B) (SEQ ID NOs: 10 & 20), which are shortened forms of a bacterial thermometer (TlpA) engineered to form heterodimers whose DNA-binding domain has been removed, and (3) small molecule-inducible dimerization domains FRB (SEQ ID NOs: 15 & 25) and FKBP (SEQ ID NOs: 14 & 24) (FIG. 1B).

Next, the in vivo phase boundaries were determined for the various RGG scaffolds and the number and size of condensates per cell were characterized. When fused to an N-terminal SZI coiled, the (RGG)₃-GFP scaffold formed an average of five condensates per cell (FIGS. 1C-1D). Addition of the TsCC(A) domain to (RGG)₃-GFP scaffolds led to fusion and formation of one to two large conden-sates per cell (FIGS. 1C-1D). To better evaluate the phase behavior of these condensates in vivo, the intracellular phase boundaries were measured for scaffolds containing various RGG domains and tags (FIG. 1E). An SZ1-(RGG)₂-GFP scaffold protein had a saturation concentration (Cₛₐₜ) of approximately 1,610 nM. Addition of a third RGG domain lowered the Cₛₐₜ to ˜600 nM, in agreement with previous in vitro findings. The TsCC(A) (RGG)₃-GFP scaffold demonstrated an even larger reduction of Cₛₐₜ to ˜29 nM, likely due to some coiled-coil homodimerization activity (FIG. 1E and FIG. 6F).

The steady-state fraction of scaffold protein that will partition to the condensate versus remain in the cytosol was determined by the Cₛₐₜ and protein expression levels. This parameter is essential because it may impact the fraction of client recruited via cognate interaction motifs. The fraction of total scaffold and client-integrated intensity in cells was measured after inducing scaffold expression. Over 95% of total TsCC(A)-(RGG)₃-GFP scaffold protein and approximately 72% of total SZI-(RGG)₃-GFP scaffold protein localized to condensates (FIG. 1F and FIG. 6G). Importantly, while an exogenously expressed mScarlet client fused to an interaction motif was diffusely localized through the cell (FIG. 6H), expression of a scaffold with the cognate protein interaction motif resulted in robust localization of mScarlet to the synthetic condensates. Over 91% of the client tagged with TsCC(B) was recruited to TsCC(A)-(RGG)₃-GFP condensates (FIG. 1G), demonstrating sequestration of a vast majority of a client protein in cells at room temperature under normal growth conditions.

The feasibility of induced cargo recruitment was also tested. The rationale was to allow a tagged client to localize and function normally in the presence of synthetic condensates under basal conditions and then rapidly induce dimerization and sequestration to the synthetic condensates. FRB was fused to the scaffold and FKBP to a client. In the absence of dimerizer, the tagged client diffused freely throughout the cytosol (FIG. 1H), and upon the addition of rapamycin (Rap), the client was quickly relocalized to the condensates (FIG. 6I). This strategy could partition approximately 50% of cargo with a time for half maximal recruitment of ˜12 min (FIG. 6J).

Collectively, both stable and inducible client recruitment to the synthetic condensates were achieved and the TsCC(A)-(RGG)₃-GFP scaffold was capable of recruiting over 90% of a client tagged with the cognate interaction motif. Based on these results, the study proceeded with the TsCC(A)-(RGG)₃-GFP scaffold for sequestering native enzymes to control cell behaviors.

### Control of Cell Behavior by Sequestering Native Enzymes

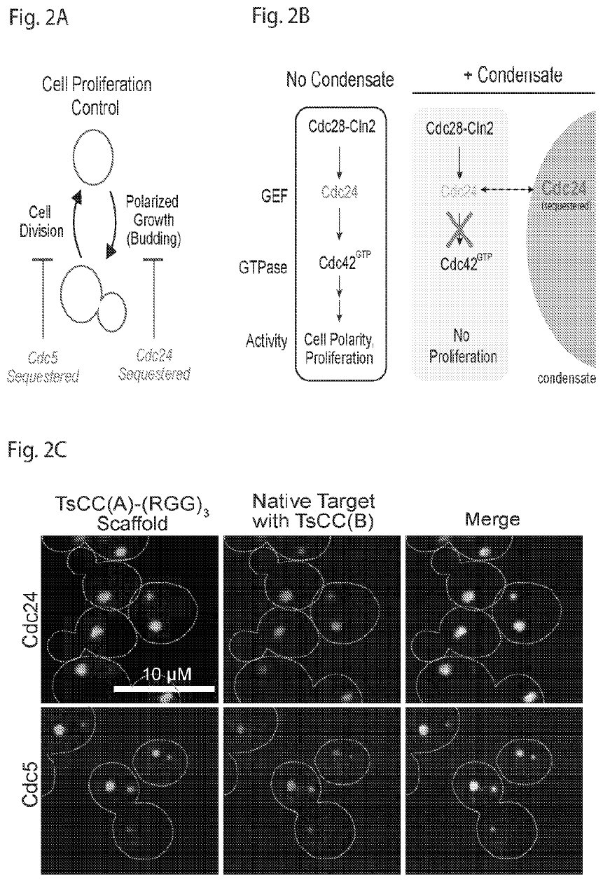

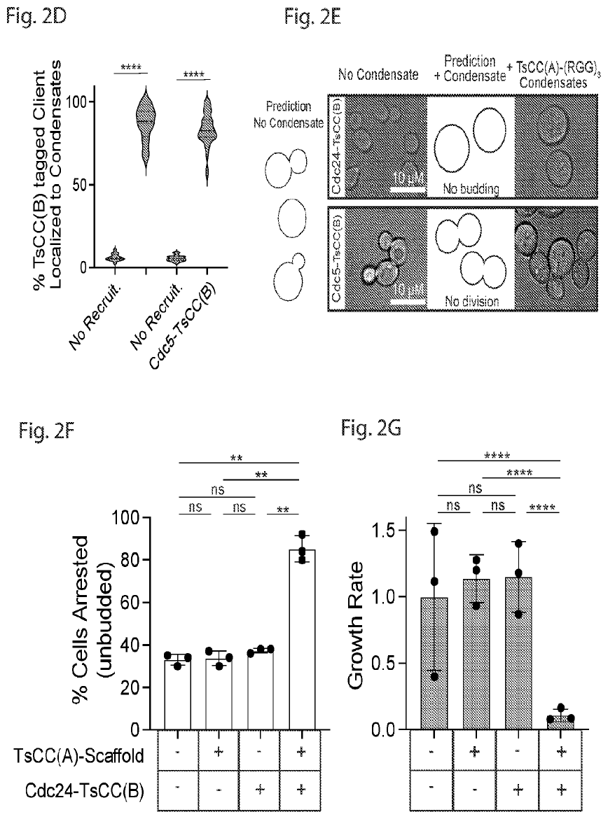

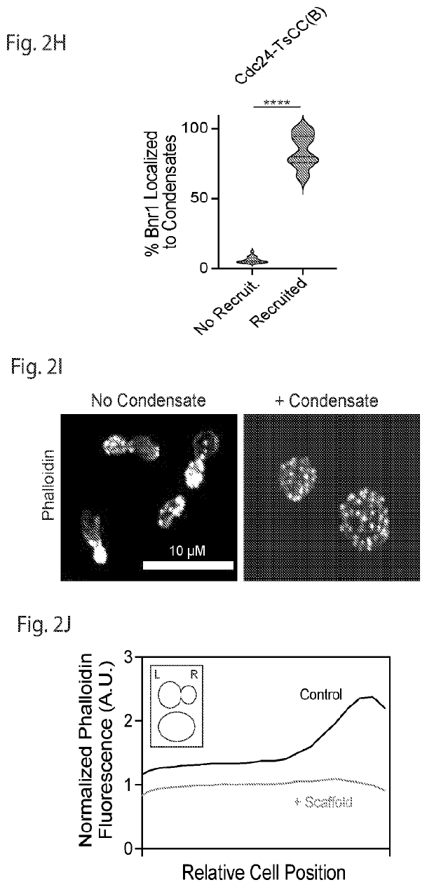

The utility of the synthetic membraneless organelle platform was tested as a protein based switch to regulate cell decision making. To modulate both sides of the cell proliferation control system, the guanine nucleotide exchange factor (GEF) Cdc24 and the kinase Cdc5 were chosen as targets for sequestration (FIG. 2A). Knockout or depletion of Cdc24 prevents polarized growth and proliferation, and loss of Cdc5 prevents cells from undergoing cell division. The hypothesis was that by tagging these proteins with coiled coils at their genomic loci, a sufficient fraction of the endogenous enzyme would be sequestered to the designer condensates, functionally insulating them and preventing pathway activity (FIG. 2B).

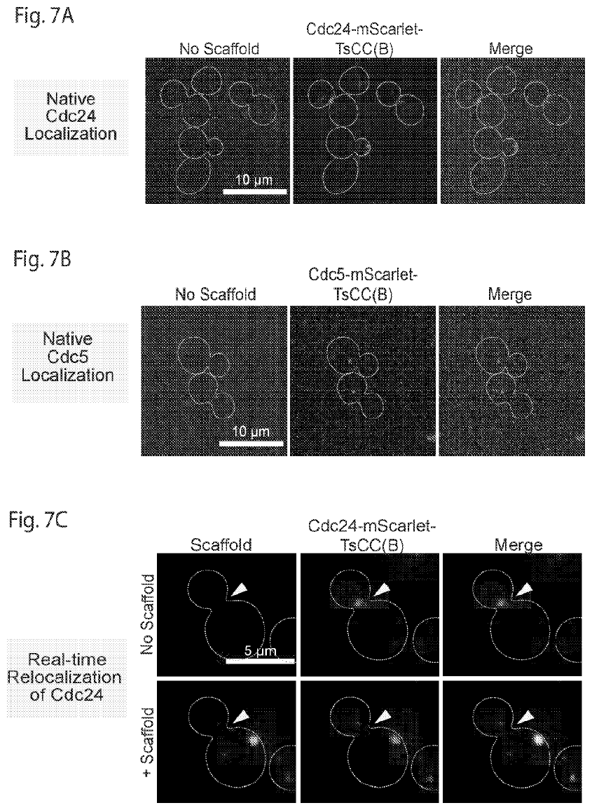

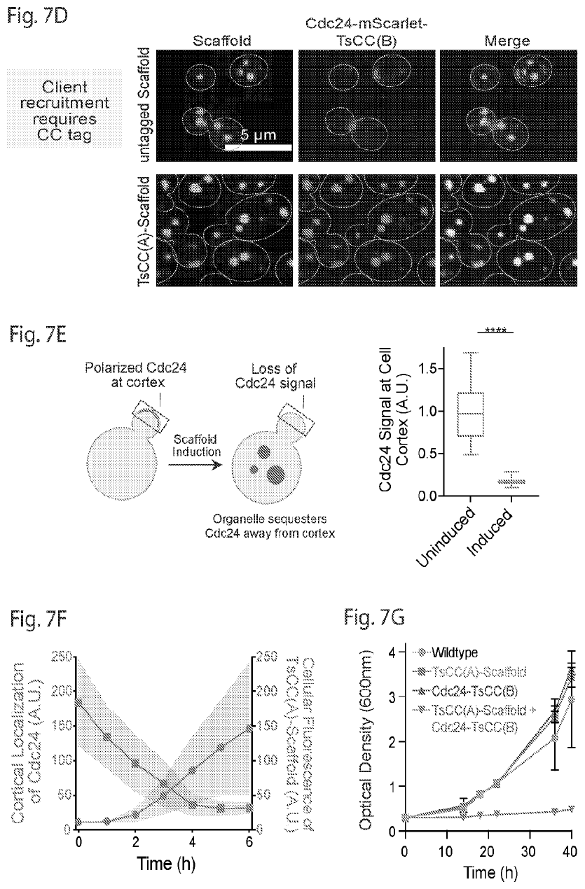

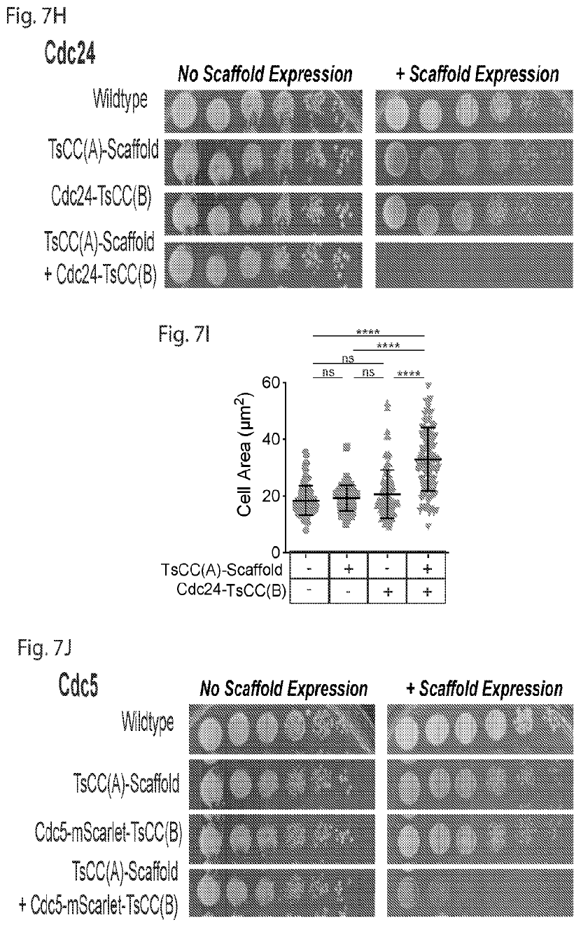

Tagging with a fluorophore and the TsCC(B) coiled coil did not affect the normal localization of Cdc24 to polarity sites like the yeast bud neck and tip or that of Cdc5 at spindle pole bodies (FIGS. 7A-7B). Natively expressed Cdc24-mScarlet-TsCC(B) was strongly recruited to condensates formed from TsCC(A)-(RGG)₃-GFP expression (FIG. 2C and FIG. 7C), but was not recruited to control scaffolds that lack the cognate coiled-coil tag (FIG. 7D). Enforced localization of Cdc24-mScarlet-TsCC(B) to the synthetic condensates competed it away from its native localization sites (FIG. 5E), and this relocalization could be observed in real time following induced expression of the scaffold (FIG. 7F). Importantly, both tagged Cdc24 and tagged Cdc5 were efficiently recruited (FIGS. 7C-7D), demonstrating that greater than 86% and 83% of the native enzymes, respectively, could be sequestered within condensates.

The behavior of cells containing endogenously tagged clients was dramatically altered by the expression of synthetic condensates functionalized with cognate recruitment tags. Cells containing tagged Cdc24 or Cdc5 grew and proliferated normally in the absence of TsCC(A) (RGG)₃-GFP condensates. However, their cell cycle control systems were blocked following the formation of condensates. Cdc24-mScarlet-TsCC(B) cells could no longer polarize or bud (FIG. 2E). Thus, localization of Cdc24 to condensates arrested cells (FIG. 2F and FIGS. 7G and 7H), leading to a nearly 12-fold drop in the rate of cell proliferation in liquid culture (FIG. 2G and FIG. 7G) and caused the cells to significantly expand in area (FIG. 7I). Importantly, only cells expressing both a tagged Cdc24 and TsCC(A)-(RGG)₃-GFP scaffold showed growth arrest: other cells behaved similar to wild-type cells. Sequestration of Cdc5-mScarlet-TsCC(B) disrupted cell division, and cells remained dumbbell shaped (FIG. 7E). As a result, sequestration and functional insulation of Cdc5 also stalled cell proliferation (FIG. 7J).

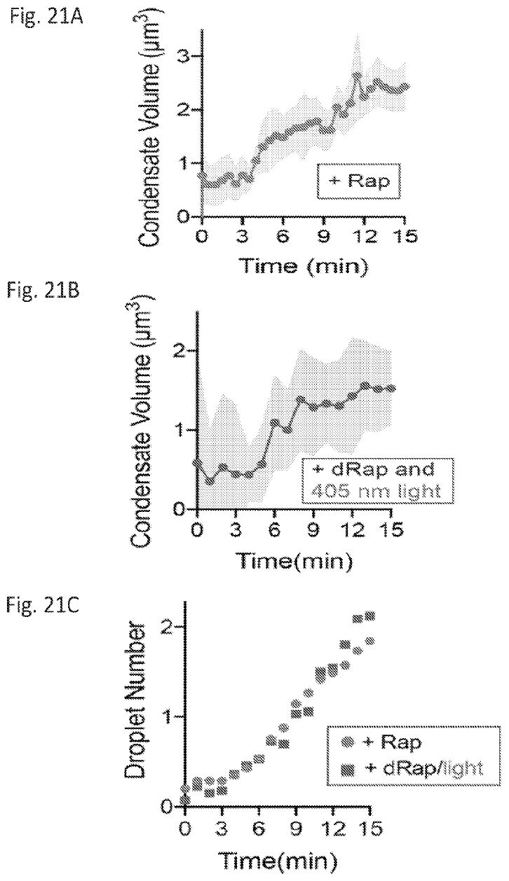

In addition to switching cell growth control, regulation of the spatial organization of the actin cytoskeleton by the synthetic condensates was also tested. To do this, a yeast formin, Bnr1, which generates linear actin cables for intracellular trafficking and polarized cell growth, was targeted. By tagging a native, constitutively active form of Bnr1 in a cell that otherwise lacks formins, 83% of it was efficiently sequestered to TsCC(A)-(RGG)₃-GFP condensates (FIG. 2H). This functional insulation of the formin prevented normal formation of actin cables and spatial polarization of the cytoskeleton (FIGS. 21 and 2J).

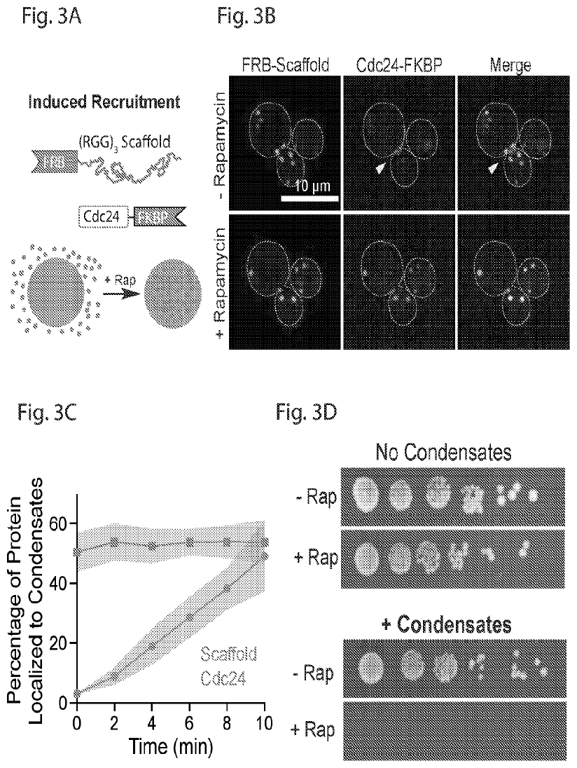

To demonstrate rapid, inducible recruitment of a native enzyme, the endogenous locus of Cdc24 was tagged with an FKBP tag in cells containing FRB-(RGG)₃-GFP condensates (FIG. 3A). In the absence of dimerizer, the native enzyme localized normally. Following the addition of Rap, Cdc24-mScarlet-FRB protein was relocalized to the synthetic condensates (FIG. 3B). Nearly 54% of the total cellular pool of tagged Cdc24 protein was sequestered to the synthetic organelle within approximately 10 min (FIG. 3C). Further, cell proliferation was effectively stalled in the presence of Rap and expressed condensates (FIG. 3D), whereas no phenotype was observed when the scaffold was expressed in the absence of dimerizer or when Rap was added to cells that lack condensates.

These results demonstrated the utility of the disordered domain-based scaffold to generate orthogonal membraneless organelles in vivo. With the addition of high-affinity coiled-coil interaction domains or inducible recruitment tags, endogenous clients were effectively sequestered and insulated in membraneless organelles. As a result, modular control over cell decision-making was demonstrated via designer compartments.

Controlled Release of Clients from Synthetic Condensates

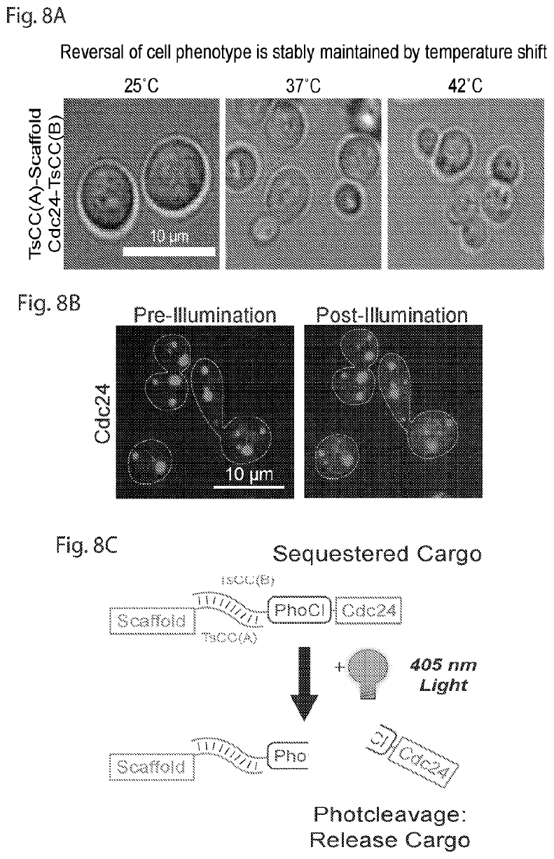

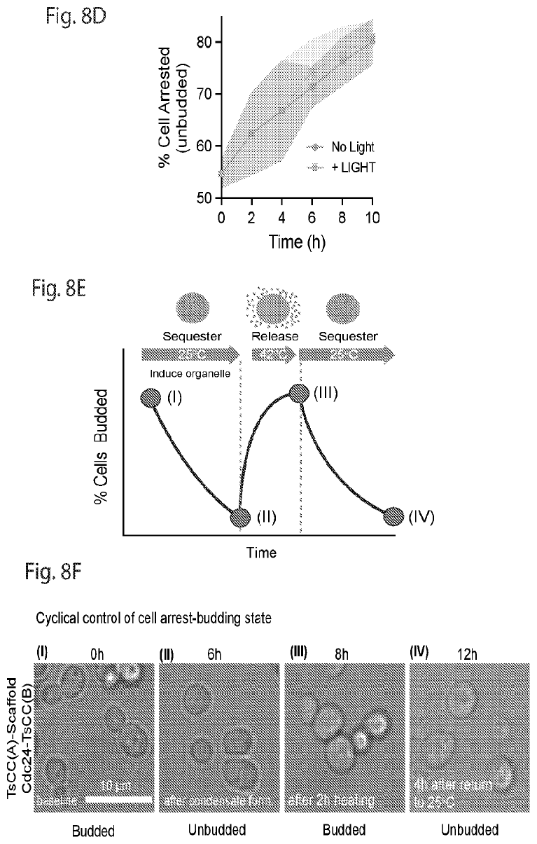

Having demonstrated efficient functional insulation of endogenous enzymes in synthetic organelles, the next step was to develop handles for controlled intracellular release. By utilizing the thermally responsive TsCC(A)-TsCC(B) coiled-coil interaction pair, it was hypothesized that client recruitment would be reversed above a critical temperature (FIGS. 4A and 4B). Cdc24-mScarlet-TsCC(B) cells were used and the cognate scaffold was expressed for 6 hours at room temperature, during which the client was sequestered and cells were arrested, while control cells that did not express the scaffold were unresponsive (FIG. 4C). Client recruitment was then reversed by raising the temperature to 37° C. or 42° C. (FIG. 4C), temperatures known to dissociate the heterodimer pair in vitro and in vivo. Strikingly, thermal induction successfully reversed the arrest phenotype of Cdc24-mScarlet-TsCC(B) cells expressing TsCC(A)-(RGG)₃-GFP condensates (FIG. 4C). This reversal was dose dependent and concomitant with a reduction of Cdc24 sequestered to the organelle (FIG. 4D): a higher temperature restored nearly wild-type levels of polarized cells. Additionally, temperature reversal of the phenotype was maintained overnight (FIG. 4C and FIG. 8A), indicating that cells could polarize at these higher temperatures, sustaining client release.

A light-based client release strategy was developed from the synthetic organelles. Optogenetic dimerization domains have been leveraged to reverse condensate clustering or to release exogenous cargoes. However, these strategies require sustained illumination and have not been demonstrated as effective in sequestering a large fraction of endogenous clients. Therefore, an optogenetic strategy was tested that would require short durations of illumination to achieve cargo release and reverse the programmed cell phenotype. In one strategy, a photocleavable domain, PhoCl, was encoded between the interaction tag and disordered domains of the scaffold and Cdc24 was fluorescently tagged to monitor light-induced client release (FIG. 4E). Following short pulses of illumination, Cdc24 quickly accumulated in the cytosol (FIG. 8B), achieving half client release from condensates in approximately 100 s (FIG. 4F). In a second strategy, a photocleavable domain, PhoCl, was encoded between the endogenous Cdc24 client and the TsCC(B) interaction tag (FIG. 8C) and the functional effect of client release was tested on switching cells between arrested and proliferative states. Condensate formation was initiated to arrest the cell cycle by sequestering Cdc24 and then a pulse of illumination was tested to see whether it was sufficient to reverse the effect. Illumination was sufficient to stably reverse the arrest phenotype, returning cells to near-normal levels of arrest, and this was maintained for up to 6 h after light exposure (FIG. 4G). Importantly, cells containing condensates and tagged client, but lacking a photocleavable domain, did not respond to illumination (FIG. 8D).

To determine whether it was possible to achieve cyclical control of client sequestration and release, a multistep proof-of-concept experiment was devised to cycle through cell proliferation and arrest. Scaffold expression was induced at 25° C. to first sequester client in condensates and arrest the cell cycle. In the next step, cargo was thermally released to reverse the imposed arrest, and, finally, the arrest would be reinduced by returning the temperature to 25° C. This strategy was tested using Cdc24-mScarlet-TsCC(B) as the client and quantified cell arrest throughout the induction and release cycles. Indeed, robust arrest was achieved following organelle induction at room temperature, followed by thermal reversal of the phenotype via heating and finally restoration of the arrest by returning the system to 25° C. (FIG. 4H and FIGS. 8E-8F).

Taken together, two distinct approaches were demonstrated for the release of native clients from synthetic organelles. The use of thermally responsive coiled coils enabled cyclical modulation of cellular control systems through client sequestration-release-sequestration and an optogenetic approach for irreversible client release from condensates, which required only a short period of illumination to stably reverse the imposed cell phenotype.

### Sequestrating CRISPR-Tagged Targets in Mammalian Cells

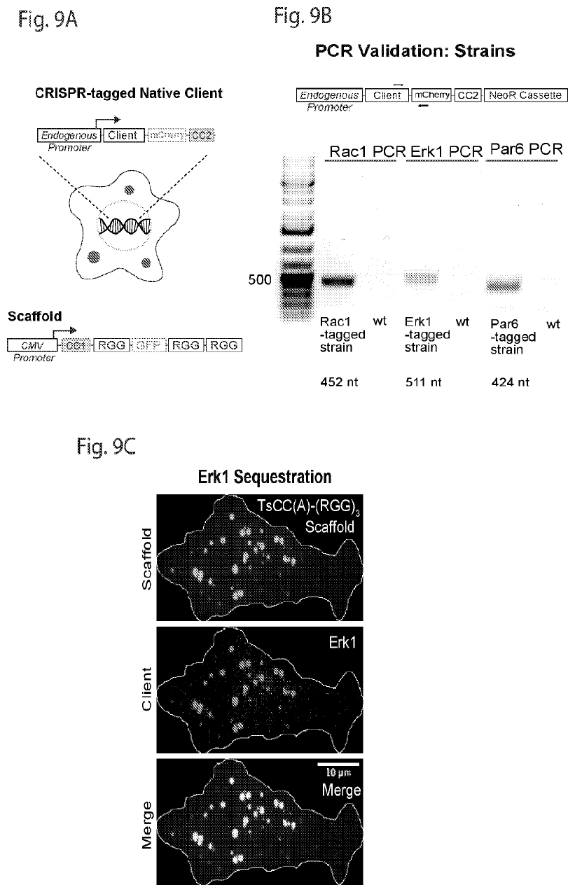

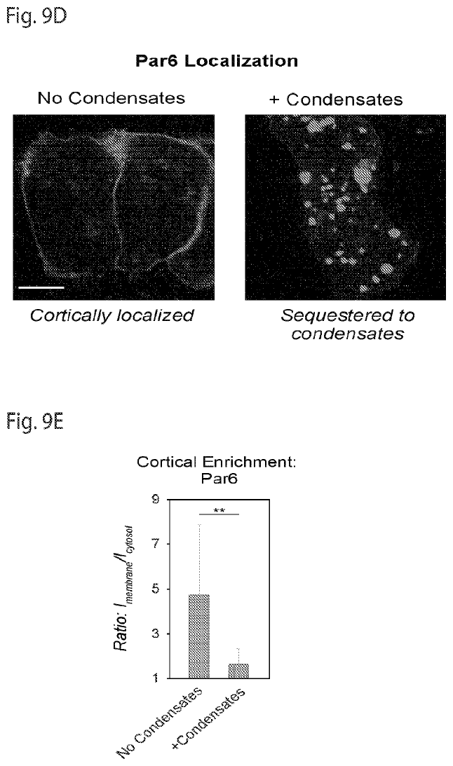

In addition to single-cell organisms with industrial applications, the platform was tested for the ability to sequester native enzymes within synthetic membraneless organelles in mammalian cells. A CRISPR knock-in approach was used to tag the 3′ end of genomic loci in U2OS cells (FIGS. 9A-9B) so that clients are expressed from their endogenous promoters. The enzyme GTPase Rac1 and the kinase ERK1, which have central roles in the control of cell signaling pathways regulating cell motility and proliferation, were selected. In migrating cells, Rac1 activity is required at the leading edge, and in proliferating cells, Erk1 is required to transmit mitogen signals from surface receptors to downstream transcriptional effectors. Cells harboring mCherry-TsCC(B)-tagged Rac1 and ERK1 showed diffuse localization throughout the cytosol, which were not recruited to condensates that lacked the correct interaction motif (FIG. 5A and FIG. 9C). By contrast, when scaffold containing the cognate TsCC(A) tag was expressed, clients robustly localized to condensates and showed substantial enrichment in the organelles relative to the cytosol (FIGS. 5A-5B). Quantitation of the fraction of enzyme protein partitioned to the condensates revealed that, on average, over 70% of the scaffold protein and nearly 50% of each endogenous tagged client were localized to the synthetic organelle (FIG. 5C). To determine whether client sequestration impacts native pathway organization, the polarity protein Par6 was tagged, which is normally enriched on the plasma membrane. After expressing synthetic condensates, Par6 was de-enriched from its native sites of localization and was sequestered within the condensates (FIGS. 5D-5E and FIGS. 9D-9E).

Taken together, these data demonstrate the utility of the synthetic membraneless organelle system for modular control of essential proteins and activities in multiple cell types. By combining the expression of a designed scaffold and tagging an endogenous genomic locus with high affinity coiled-coil interaction motifs, it is feasible to impose cell behavioral states in real time through functional sequestration of enzymes to designer membraneless compartments in cells.

### Discussion

Protein engineers have only recently begun to target the self-assembly of polypeptides into mesoscale membraneless compartments expressed in living cells. Concurrently, metabolic engineers have leveraged these and other compartments to cluster exogenous enzymes to produce novel products. Cellular engineers interested in programming cellular behaviors and decision making embed new molecules into cells that function as receptors or switches to augment or redirect native behaviors. Herein, the toolkit for cellular engineering was expanded by constructing a designer membraneless organelle system from disordered proteins that is capable of efficient client sequestration and release. When recruited to synthetic condensates, a targeted client is insulated from its native pathway, thereby generating predictable switching of cell behavior. Additionally, controlled release of sequestered clients from synthetic organelles was demonstrated using optical and thermal induction, which complement existing strategies such as light regulated condensate disassembly. The platform is generalizable to control of a variety of native components and pathways, and its application was demonstrated in multiple cell types, including cells used for bioproduction and for mammalian tissue culture.

Cells enhance pathway flux and selectivity by enforcing the proximity of pathway components. This can be achieved by binding the components to platforms, such as macromolecular scaffold proteins, or by anchoring them to the plasma membrane. Colocalization increases the effective concentration of proteins and reduces interactions with other competing factors in the cells. Additionally, cells achieve even higher levels of specificity through physical compartmentalization, trafficking components into membrane bound organelles such as the nucleus or lysosome. Although these are attractive strategies for reengineering subcellular reaction schemes, they have a number of draw backs. It is currently not feasible to rewire native lipid metabolism to create a new orthogonal compartment bounded by a lipid bilayer membrane. Also, although assembly of enzymes and substrates onto a single nanometer size macromolecular scaffold protein can enhance flux, this reaction scheme is quite sensitive to scaffold protein concentrations and titration effects, and, thus, fluctuations in protein levels may lower reaction efficiency. Protein based compartments offer a number of potential solutions to these engineering challenges.

Construction of synthetic subcompartments inside a cell from protein based materials relies on polypeptides that assemble into nanocapsules or mesoscale condensates. At the nanoscale, exogenous assemblies of encapsulins or designer protein cages provide one avenue for targeting components. However, these compartments are tens of nanometers in diameter, limiting their cargo capacity. Further, their highly restrictive permeability often prevents the diffusion of macromolecules in and out. At the microscale, multivalent binding proteins can undergo complex coacervation, or disordered polypeptide polymers can self-assemble to form mesoscale condensates. Native membraneless organelles, such as P granules and the nucleolus, contain disordered protein components that condense and contribute to proteinaceous compartment self-assembly. These dynamic liquid-like compartments demonstrate selective composition and restricted permeability but are also highly porous to molecular and macromolecular clients. An important feature of designer protein condensates is that they can achieve large sizes and therefore offer high payload capacities. Additionally, the dimensions and permeability of protein condensates are tunable, for example, by increasing protein polypeptide length or expression levels above the saturation concentration. Therefore, membraneless organelles provide a means to scale the output of reactions localized to the compartment, something that is harder to achieve via endogenous membrane-bound organelles.

Disordered protein sequences have been leveraged to generate synthetic liquid-like condensates in living systems. Examples in model eukaryotic culture systems include Corelets, OptoDroplets, REPS and SPLIT among others. More recently, resilin-like polypeptide sequences have been redesigned to assemble designer condensates in prokaryotic systems. In the present disclosure, a disordered RGG domain from Laf-1 was leveraged, whose sequence displays upper critical solution temperature behavior, and phase separation can be tuned by sequence mutation or by controlling domain valency and is amenable to engineering cytosolic condensates. The Cₛₐₜ was optimized by changing RGG polymer valency and through interaction motifs, generating a robust condensate system that partitions more than 90% of the cellular pool of scaffold to the synthetic organelle in budding yeast. Many phase-separating proteins, including those of the FET family, possess RNA-binding RGG domains, which have been shown to enhance LLPS alone and in the presence of RNA52. Although the idea that the RGG platform may still interact with RNAs cannot be excluded, it does not require RNA to phase separate in biochemical reconstitution experiments, and the temperature-dependent phase behavior in cells matches behaviors from in vitro experiments (FIGS. 6B-6C).

There are a variety of strategies to enrich clients in synthetic compartments, although there are strengths and limitations of each approach. Similar to localization motifs used in cells, short coiled-coil sequence pairs can be used to target a client protein to a disordered scaffold. Alternatively, a disordered sequence can be appended directly to a protein of interest to target its partitioning to the scaffold only in the condensed state. A challenge of fusing low-complexity polypeptide sequence to a native protein is that it may alter stability or endogenous interactions and functions. Because the goal of this study was to target essential proteins at their native encoding genomic loci, coiled-coil interaction pairs were chosen for use. These high-affinity tags have been shown to be functional and orthogonal in vivo in other cellular-engineering studies, and it was demonstrated herein that tagging of the GEF Cdc24 or the kinase Cdc5 with coiled-coil interaction domains does not disrupt localization and essential activities.

Additional challenges to which the system is also subject are design considerations, including the intrinsically disordered region/folded protein ratio of the scaffold and the limitations to protein expression inherent to in vivo studies. Because coacervation is relied on to form condensates capable of sequestering high levels of native clients, the scaffold must necessarily be expressed at levels well above its Cₛₐₜ. In yeast, GAL1 promoters lead to high expression levels, allowing up to 90% client partitioning and control over cell behavior to be achieved. However, in transient transfections of mammalian cells, as high a level of scaffold expression is not achieved and only approximately 80% scaffold partitioning to condensates is obtained. This reduced partitioning relative to expression in yeast helps explain the lower client partitioning in mammalian cells. Future work that enhances scaffold expression, for example, via multicopy viral integration, would ensure higher fractional client partitioning. Nevertheless, using the current iteration of the platform transiently expressed in mammalian cells, substantial amounts of native enzymes Rac1 and Erk1 were recruited and Par6 sequestered, insulating it from its normal localization along the cell cortex.

Further, one must also consider that client size, subcellular localization and stoichiometry relative to the disordered sequences of the scaffold may affect the levels of client partitioning. Efficient functional insulation of the GTPase Cdc24 and kinase Cdc5 was demonstrated herein. Efficacy is likely high because the substrates of these enzymes are dozens of kilodaltons and therefore do not easily diffuse inside the condensates. Additionally, the normal subcellular positioning of Cdc24 to the plasma membrane and Cdc5 to spindle pole bodies likely enhances the functional effect of sequestration on shutting down pathway activity. It may be more challenging to insulate metabolic enzymes whose reactants and products are small molecules that more readily diffuse in and out of synthetic condensates. One additional unknown is whether client sequestration to synthetic condensates will exhibit an inverse size dependence at some critical size. In the current study, clients whose molecular weight is greater than 100 kDa of folded domains were effectively sequestered when including recruitment tags and fluorophores.

Other inducible sequestration or inducible knockdown systems are often less effective for achieving functional knock-down of highly expressed cytoplasmic proteins, and anchoring targets to native structures, such as the plasma membrane, endoplasmic reticulum or Golgi membranes. Additionally, achieving reversibility of these systems by small molecular washout is challenging. RNA interference (RNAi) strategies, although useful, can be incomplete and quite slow, taking days to sufficiently clear preexisting transcripts. Auxin-induced degradation systems overcome the time limitations of RNAi, enabling knockdown of protein levels within tens of minutes to hours. However, these systems are difficult to reverse, often requiring extensive washing out of the small molecule and multiple rounds of cell division to restore protein levels. The synthetic membraneless organelle system developed herein has a number of advantages. It is orthogonal, offers a high payload capacity, is capable of ultrahigh sequestration of targeted clients and demonstrates controlled client release, readily reversing the cell activity state both by thermal and optical means.

Unique features of this condensate platform include regulatory handles for thermal and optical control of client release. Using thermally responsive coiled coils as interaction motifs, reversal of client recruitment to synthetic condensates can be achieved by transient shifts to elevated temperatures of 37-42° C. Although yeast grow normally at 37° C., maintaining temperatures as high as 42° C. for long periods of time is not advisable and will produce a heat shock stress response. Additionally, although temperature transients are possible through ultrasound heating of mammalian cells, we would largely recommend thermal client release only for yeast. However, light-based client release is highly effective in both yeast and mammalian cells and has a number of clear applications for cell biology and cellular engineering. A simple experimental setup would be to express the disordered scaffold along with an exogenous client that one would like to release for the regulation of cellular behavior or cell fate and to illuminate the system to achieve sustained client release on the timescale of minutes. For example, sequestered signaling enzymes or transcription factors could be rapidly released to modulate a cellular decision. In effect, this system can be considered an intracellular drug delivery or controlled release platform, one in which the kinetics of client accumulation in the cytoplasm would be substantially faster than inducible transcription and translation.

A new strategy is disclosed herein for the programmed control of cellular decision making by modular targeting of cellular machinery to synthetic membraneless compartments. Near complete targeting and insulation of endogenously expressed enzymes following organelle induction was demonstrated. Sequestration to the designer organelles blocks its native localization and function, thereby switching off cell polarity and proliferation control systems in a single-cell system with industrial applications. Using thermosensitive interaction motifs or photocleavable domains, effective and cyclical reversal of client recruitment is shown as well as subsequent reversal of cellular phenotypes. Further, this platform was extended to mammalian cells and efficient client recruitment and insulation from native targeting sites was shown, demonstrating the membraneless organelle system as generalizable across cell types and applications. This study revealed that de novo compartmentalization of native enzymes can be used to engineer cellular systems capable of responding to specific stimuli with predictable outcomes. This synthetic organelle approach can be leveraged as a hub to insulate and rewire native biochemical pathways to reveal principles of pathway organization or as a protein switch based for cellular engineering.

### Example 2: Lentivirus Delivery System, Cell Lines, Drug-Inducible and Optical Control

Synthetic proteinaceous organelles were generated in mammalian cells for applications in biomedicine, including cellular engineering. The technology disclosed herein was expanded to include: (1) a lentivirus delivery system: encoding a disordered protein capable of self-assembly into micron-size condensates upon transduction into human cells, (2) generation of stable monoclonal cells lines capable of long-term expression of synthetic organelles, (3) drug-inducible, temporal control of condensate formation in cells, (4) expression of synthetic organelles in primary and undifferentiated cell types used in reprogramming, and (5) demonstration of optical controlled release of client protein from synthetic organelles in mammalian cells.

### Construction of Lentiviral Vectors for Efficient Transduction and Expression of Synthetic Organelles in Human Cells

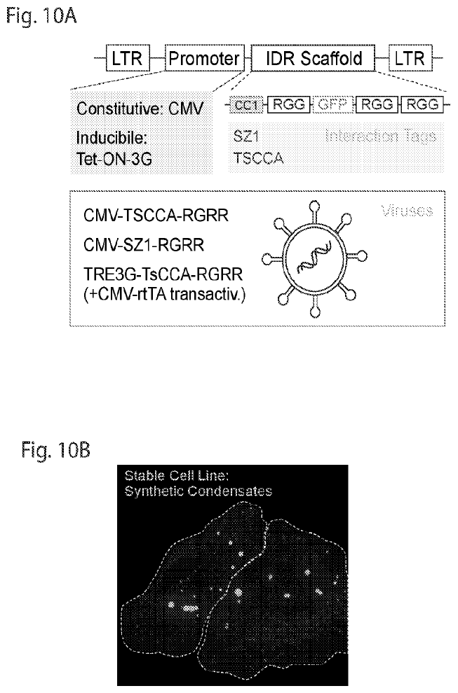

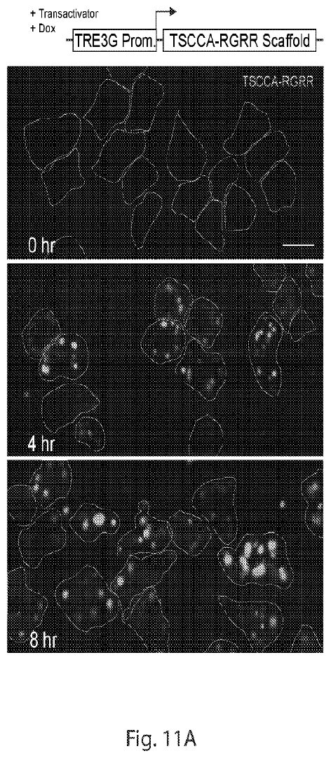

Herein, a disordered protein scaffold was transiently transfected into human transformed tissue culture cell lines to form micron-size protein condensates capable of functioning as synthetic organelles. The efficiency and functionality of this modular platform was further expanded for mammalian cell engineering by generating a toolkit of lentiviruses. Lentiviruses are a genus of retroviruses that allow DNA delivery to a wide variety of human cell lines, achieving more homogenous expression profiles compared to standard liposomal DNA transfection. Further viral transduction facilitates delivery and expression of orthogonal genetic components in primary human cell lines that often cannot be achieved by other methodologies. An optimized set of disordered Laf-1 RGG constructs that achieve high expression and partitioning into synthetic condensates in cell culture, were inserted into viral transfer vectors (FIG. 10A). This included RGG-GFP-RGG-RGG tagged at the N-terminus with either a SynZIP1 or TsCCA coiled coil, tags used for client recruitment to the synthetic organelles. Further, viral transfer vectors were generated that contain the IDR scaffold with either a constitutive CMV viral promoter or a Tet-ON-3G drug-inducible promoter. By co-transfecting the plasmids with packaging and envelope plasmids in HEK293T cells, high-titer lentiviruses were generated.

### Generation of Stable Monoclonal Cell Lines Expressing the Synthetic Organelles

To date, stable, long-term expression of synthetic condensates has not been demonstrated. This is an essential step for demonstration of orthogonality and for regenerative medicine in which implanted cells survive for a duration of weeks to months. A stable monoclonal cell line was generated that expresses the synthetic condensate platform disclosed herein, which can be used for additional rounds of genetic engineering or CRISPR screening. Following viral transduction of U2OS cells using a lentivirus encoding CMV-TsCCA-RGG-GFP-RGG-RGG and a puromycin resistance cassette, cells were grown in the presence of drug for one week to select for genomic integration. Cells were then detached, and high expressing cells (GFP) were sorted as single cells into 96-well plates using a flow cytometer. Monoclonal cultures were grown for 18-21 days until reaching 70% confluence, trypsinized, expanded and frozen as aliquots in liquid nitrogen. Stable-high expression of GFP condensates was determined by confocal microscopy. These stable lines showed near 100% formation of condensates across cells and maintained steady high-level synthetic organelle expression for 37 days (FIG. 10B). These monoclonal cultures are capable of further transfection with exogenous cargo proteins, tagged with the TsCCB coiled coil, for targeting to the synthetic organelle.

### Drug-Inducible Formation of Synthetic Organelles

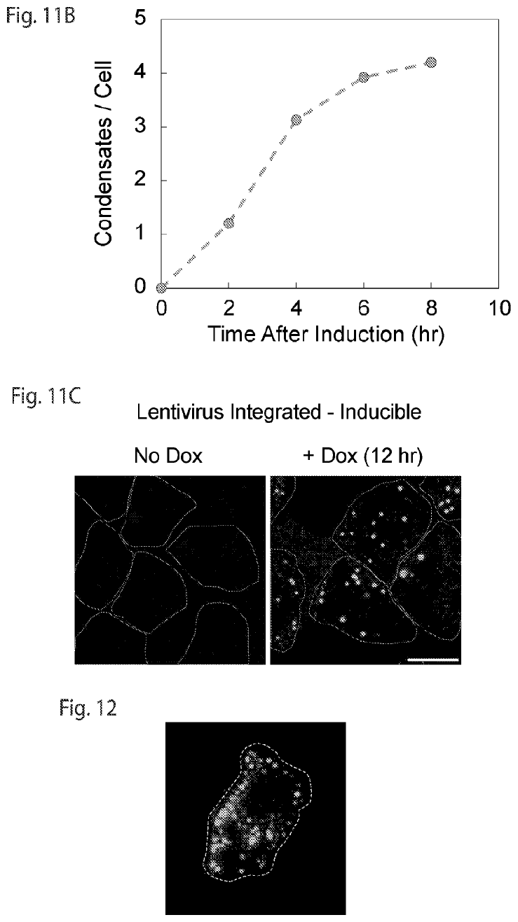

Expression of recombinant proteins in cell lines can cause toxicity and reduce proliferation. This toxicity is particularly an issue for intrinsically disordered proteins that self-assemble into condensates. To overcome this challenge, expression plasmids were constructed for the synthetic organelle platform, in which transcription is repressed under normal culture conditions and organelle formation is stimulated by the addition of a drug. Plasmids were built for transient transfection and lentiviral transduction, which contained the tetracycline responsive TRE3G promoter whose activity can be upregulated in the presence of the rtTA transactivator (expressed from a separate plasmid via CMV promoter) and doxycycline, and the scaffold construct, TsCCA-RGG-GFP-RGG-RGG. HEK293T cells were transiently transfected with the TRE3G-TsCCA-RGRR and the CMV-rtTA plasmids. After 2 days, transfection cells showed essentially no expression of the GFP-tagged scaffold and no formation of synthetic condensates (FIG. 11A). Upon addition of doxycycline to the culture media, fast assembly of synthetic organelles was observed. Condensates were visible within 2 hours of drug addition and were present in most cells by 6-8 hours, showing a temporal rise in number, size and intensity (FIG. 11B). Separately, U2OS cells were transduced with a set of lentiviruses to generate stable cell lines that displayed drug-inducible organelle formation. Lentiviruses separately contained TRE3G-TsCCA-RGRR and the CMV-rTTA. Transduced cells had no condensate expression while passaging in culture. Upon overnight addition of doxycycline, excellent expression of condensates was observed in nearly all cells (FIG. 11C). Altogether, tight chemogenic regulation of synthetic organelle formation in human cells lines was demonstrated. Among other things, these tools are valuable for applications in which native enzymes have already been CRISPR tagged with TSCCB, enabling drug-inducible sequestration of targets to the synthetic organelle.

### Demonstration of Synthetic Organelle Formation in Primary and Undifferentiated Human Cell Lines, for Applications in Regenerative Medicine

A major hurdle in the engineering of human primary cells is the efficient transfer of exogenous DNA. Viral transduction or LNP delivery of DNA is required to achieve high expression of recombinant proteins in these cells. Leveraging the lentiviral condensate-expressing toolkit disclosed herein (FIGS. 10A-10B), synthetic organelles were generated in a variety of primary human cell lines, or in cells that have yet to undergo full differentiation.

Human foreskin fibroblasts are derived from the foreskin of a neonatal male and are a workhouse of stem cell research. They are commonly used for reprogramming to a naïve state with iPSC methodologies, and subsequent differentiation into a cell type of interest for individualized cell replacement therapies. It was investigated whether these cells would replace the synthetic condensates, for later application to the sequestration and release of Yamanaka factors, a set of transcription factors sufficient to reprogram cells to pluripotency. These primary fibroblasts were transduced, and robust condensate formation was observed within 2 days of infection (FIG. 12). Additionally, the synthetic organelles were deployed as a hub to control the motility of neutrophil-like HL60 cells, which have been used to study myeloid differentiation. In vitro they resemble promyelocytes but can be induced to differentiate terminally in vitro. Upon differentiation they are capable of highly efficient chemotaxis. Undifferentiated HL-60 cells, which appear round in shape, were transduced and formation of condensates was observed in the thin rim of cytoplasm around the cell nucleus. Together, these data demonstrate the feasibility of forming synthetic organelles in a variety of human cell types using the lentiviral scaffold-expressing toolkit. These tools are broadly useful for cellular engineers interested in modulating the function and fate of immune cells for clinical therapies or cell replacement strategies for regenerative medicine.

Controlled-Release of Client Proteins from Synthetic Organelles in Human Cell Lines, Using Light

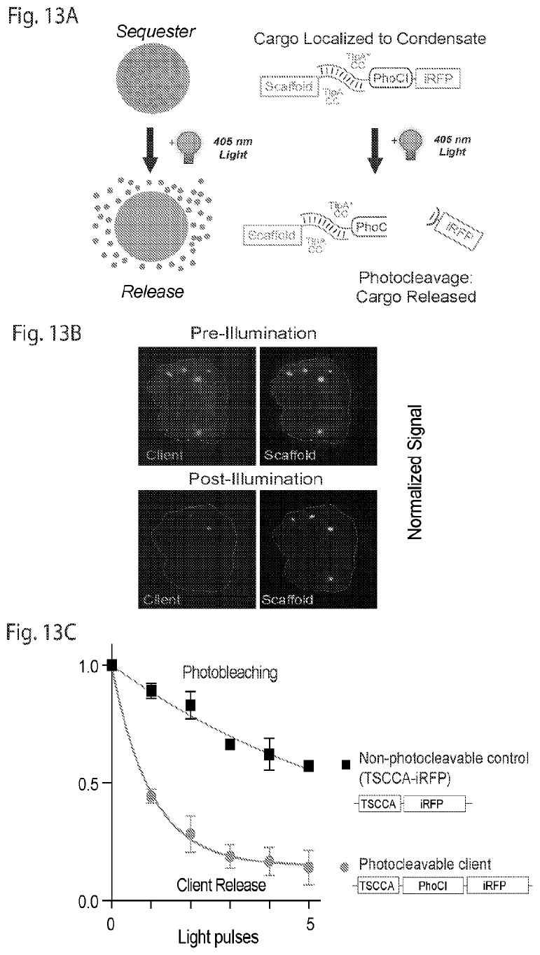

Herein, optical release of client proteins sequestered to synthetic organelles in the industrial model eukaryotic cell, Saccharomyces cerevisiae, was demonstrated. It was investigated whether this light-regulated controller would function in mammalian cells for stimulated release of cargos from synthetic condensates. The photocleavable protein PhoCl-2f, was cloned into both scaffold and client constructs, between the domain of interest and the coiled coil interaction tag. Both SZI and TsCCA coiled coil pairs attached to the scaffold were tested for client recruitment, and pulses of 405 nm laser light were used to induce client release (FIG. 13A). Client release from synthetic organelles was monitored with the far-red protein iRFP713. Tethered TsCCA-iRFP client was quickly released from TsCCA-RGRR synthetic condensates expressed via transient transfection of U2OS cells (FIG. 13B). Release kinetics showed over 90% reduction of fluorescent client signal from condensates within 10 minutes, and a t½ of release of less than 1 minute (FIG. 13C). The reduction in client signal was not caused by photobleaching as a control construct showed less than a 50% drop in condensate localized intensity over the first 6 light pulses. These results provide a novel paradigm for rapid, stimulated intracellular ‘drug-release’ of select enzymes from synthetic organelles with clear applications in sequestration and release of clinically relevant protein therapeutics.

### Example 3: Protein Condensate Formation Via Controlled Multimerization of Intrinsically Disordered Sequences

### Materials & Methods

### Protein Expression and Purification.

Plasmids encoding N-terminally 6×His-tagged RGG constructs with coiled coil tags and 6×His mCherry-FRB were transformed into BL21(DE3) E. coli cells (Thermo Fisher Scientific: Waltham, MA). Cultures were grown in Luria Broth (LB) containing kanamycin at 37 C to an OD₆₀₀ of 0.6 to 0.8 and expression was induced by 0.5 mM Isopropyl β-D-1-thiogalactopyranoside (IPTG) at 16 C overnight. Cell pellets were collected and stored at-80 C.

### RGG-FKBP and RGG-FRB Purification:

Bacterial cell pellets were thawed, resuspended in lysis buffer (50 mM HEPES, pH 6.8, 1 M NaCl, 20 mM Imidazole, 1 mM β-mercaptoethanol) containing Complete EDTA-free protease inhibitor cocktail (Roche: Mannheim, Germany), and lysed by a total of 3 minutes of sonication at 50% power, using a Branson Sonifier. Lysates were clarified by centrifugation at 13,000 rpm (20,064×g) for 20 min in an F21S-8x50y rotor (Thermo Fisher Scientific) at 37 C, and incubated with 0.5 mL of Ni-NTA beads (Thermo Fisher Scientific) and at room temperature for 1 hr. Beads were then washed three times with 10 mL of lysis buffer. Proteins were eluted by addition of lysis buffer containing 500 mM imidazole and 1 mM DTT. Elutions were diluted to 3 mg/mL in lysis buffer containing 1 mM DTT and dialyzed overnight into single RGG storage buffer (500 mM NaCl, 20 mM HEPES, pH 6.8, 1 mM DTT) using 10 kDa cutoff Slide-A-Lyzer membrane cassettes (Thermo Fisher Scientific). Proteins were concentrated by centrifugation in 4 ml Amicon filter concentrators with 10 kDa cutoff (Millipore Sigma: Burlington, MA). 1 mM TCEP added prior to snap freezing and storage at-80 C.

Purification of Tandem, RGG-RGG, Fluorescent Tracer (RGG-GFP-RGG), and 6×his-RGG Domains Tagged with Coiled Coil Dimerizers (P3-6, SZ1/2), and 6×his-mCherry-FRB:

Pellets were thawed and resuspended in lysis buffer. RGG polypeptides that included P3-6 or tags and mCherry-FRB were resuspended in lysis buffer containing 50 mM Tris-HCl, pH 8.5, 1 M NaCl, 20 mM Imidazole, 1 mM β-mercaptoethanol, and a dissolved tablet of protease inhibitor cocktail (Roche). RGG constructs with SZ1/2 tags were treated similarly but lysis buffer contained 20 mM HEPES PH6.8. Cells were lysed, cleared by centrifugation, and incubated with Ni-NTA beads as above. Beads were then washed in three column volumes of lysis buffer and eluted with lysis buffer containing 500 mM imidazole and 1 mM DTT. Elutions were diluted to 3 mg/mL in lysis buffer containing 1 mM DTT as above and dialyzed overnight into storage buffer: Constructs with P3-P6 and mCherry-FRB: 1 M NaCl, 20 mM Tris-HCl, pH 8.5, 1 mM DTT: constructs with SZI, SZ2: 1 M NaCl, 20 mM Tris-HCl, pH 6.8, 1 mM DTT. Proteins were concentrated by centrifugation in Amicon filter concentrators with a 10 kDa cutoff before addition of 1 mM TCEP and storage at-80 C. In all cases, protein concentrations were determined by A280) and Bradford assay (BioRad).

### Turbidity Measurements:

Protein aliquots were thawed and solubilized at 50 C and then diluted to 6 μM in buffer to adjust to a final salt concentration of 150 mM in 20 mM, Tris-HCl pH 8.5. SYNZIP tagged constructs were similarly adjusted to 10 μM in 150 mM NaCl, 20 mM HEPES pH 6.8. 60 μL of protein was added to quartz. microcuvettes (10 mm path length) (Starna Cells, Inc. Atascadero, CA). Cuvettes were inserted into a Cary 3500 UV-Vis spectrophotometer controlled by an Agilent multizone peltier temperature controller (Agilent Technologies: Santa Clara, CA). For kinetics tests of rapamycin-induced dimerization of FRB and FKBP tagged RGG constructs, rapamycin (Sigma-Aldrich: St. Louis, MO) was spiked into the protein mixtures to a final concentration of 10 μM and absorbance at 600 nm was measured over time. For mapping temperature dependent phase separation, protein mixtures were applied to quartz cuvettes preincubated at 50 C. Cuvettes were then inserted into the pre-heated spectrophotometer, set to 50 C, and samples were cooled to 5 C at a rate of 1 C per min while measuring absorbance at 600 nm.

### Imaging of Protein Condensation In Vitro:

Fluorescence microscopy imaging of protein droplet formation was performed at ambient temperatures (approximately 22° C.) on an Olympus IX81 inverted confocal microscope (Olympus Life Science: Tokyo, Japan) equipped with a Yokogawa CSU-XI spinning disk, Mercury lamp, 488 and 561 nm laser launches, iLas targeted laser system for photobleaching, and an iXon3 EMCCD camera (Andor: Belfast, UK). Multidimensional acquisition was controlled by MetaMorph software (Molecular Devices: Downingtown, PA). Samples were illuminated using a 488 nm laser and imaged through a 100x/1.4 NA oil-immersion objective. To image in vitro droplet formation, proteins were thawed at 50 C and diluted to 4 μM in a buffer containing 150 mM NaCl and 20 mM Tris-HCl (pH 8.5 unless otherwise specified), and placed custom fabricated acrylic gasket chambers adhered to glass cover slips.

### Chemical Dimerization.

The fluorescent tracer, RGG-GFP-RGG, was present in the protein mixture at 0.1-0.2 μM to track condensation in the 488 nm channel. Chamber wells had been previously passivated overnight in solution of 10 mg/mL BSA (Thermo Fisher Scientific) at room temperature and then rinsed with sterile ddH₂O immediately prior to use. Condensate formation of FRB and FKBP tagged proteins was induced by addition of rapamycin (Sigma-Aldrich) at a final concentration of 10 μM per reaction. Tandem RGG constructs in the same buffer conditions noted above formed condensates in the absence of additional components. Condensate formation was monitored by timelapse imaging with brightfield transmitted light and via 488 nm fluorescence. FRAP experiments were conducted on the same microscope using 405 nm light from an iLas targeted laser system. For photobleaching of internal regions of droplets, ROIs of similar sizes were selected and bleached. For photobleaching of whole droplets, a circular ROI encompassing an entire droplet was selected and photobleached as above.

Optical Uncaging of dRap for Light Induced Condensation.

Proteins mixtures were assembled in a dark room. Proteins were diluted to 10 μM in a reaction in a buffer containing 150 mM NaCl and 20 mM HEPES, pH 6.8, and supplemented with 5 μM dRap. Each molecule of dRap, upon uncaging, liberates two molecules of Rap. The protein mixture was then encapsulated inside cell-size water-in-oil emulsions by repeated pipetting of a 1 μL of aqueous phase within 50 μl of a 5% (w/v) mixture of Cithrol DPHS (Croda, Inc. Edison, NJ) dissolved in mineral oil (Sigma Aldrich). This emulsion mixture was then applied to wells in custom imaging chambers that had been pre-treated overnight with mineral oil. Emulsions were allowed to settle for 20 min in the dark. To induce condensation, emulsions were subjected to 30 sec total of continuous 405 nm light from a Mercury lamp applied in steps through the Z-planes. Droplet formation inside emulsions was then monitored via timelapse microscopy. Occasionally, emulsions would drift and the field of view was re-centered manually between imaging intervals.

### Yeast Procedures:

Standard methodologies were followed for all experiments involving S. cerevisiae. For Rap and dRap mediated droplet assembly, a tor 1-1 fpr1Δ::KANMX6 strain in the BY4741 genetic background was used. All other yeast strains were of the YEF473 genetic background. RGG constructs with coiled coil or FRB and FKBP tags were cloned downstream of a galactose inducible GAL1 promotor and integrated into the yeast URA3 locus using the Yiplac211 integrating vector or LEU2 locus using the Yiplac 128 integrating vector by linearizing plasmid with EcoRV just before transformation. All yeast transformations were performed by the standard lithium acetate method.

To induce expression of RGG constructs in yeast, cells were first grown to saturation overnight in liquid YPD media in a 25 C shaking incubator. Cells were then washed three times in sterile water and diluted in YP+2% Raffinose and incubated in a 25 shaking incubator for 6 to 8 hours. Finally, yeast cells were diluted to an OD₆₀₀ of 0.3 in YP+2% Galactose induction was allowed to proceed overnight in the same shaking incubator or for hours on a microscope slide to track scaffold induction and cargo recruitment in the same cells. Final OD₆₀₀ of cultures used for experiments were between 0.4-0.8.

### Image and Data Analysis: Quantitation of In Vitro Protein Condensate Formation.

Image segmentation in ImageJ was used as follows: 2D maximum intensity projections in the 488 nm channel (RGG-GFP-RGG tracer) were generated for each time point and converted to an 8-bit images. A binary mask was generated by automated thresholding with the Internodes algorithm, objects were cleared from the boundary, and a watershed function used to split objects. The particle analysis function in ImageJ was used to segment condensates. This provided droplet number and areas from maximum intensity projections at each timepoint. Conversion of droplet areas to volumes was performed assuming a spherical shape.

FRAP experiments were analyzed by placing an appropriately sized ROI over the photobleached area of each droplet. The fluorescence profile over time for each ROI over time was recorded and the maximum value prior to photobleaching was set to one. Results from FRAP experiments for each type of droplet were then pooled and the average recovery is shown with standard deviation.

Analysis of condensate formation in cells was performed in similar manner in ImageJ. Time-lapse images were converted to maximum intensity projections. Individual objects (cells) were first cropped to facilitate condensate segmentation. Images were corrected for bleaching using an exponential fit. Condensate masks were generated from 8-bit images thresholded by the internodes algorithm. In a small number of cases, despeckling was required prior to thresholding. Cells that could not be reliably thresholded were excluded from analysis. After generating a mask, the particle analysis was performed in ImageJ as above to generate object number and size. Number of droplets are reported as generated by this analysis and volumes were calculated from the 2D areas assuming objects are spherical.

### Results

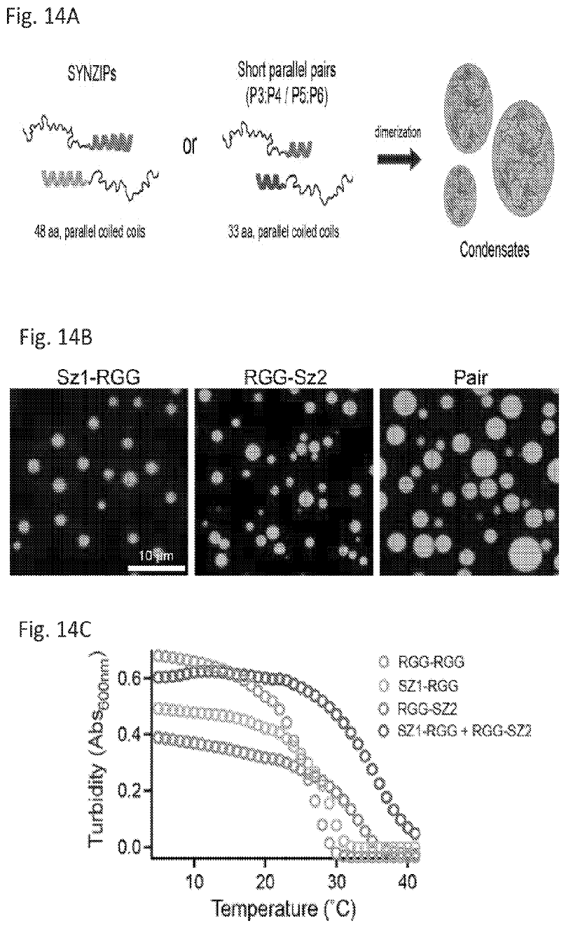

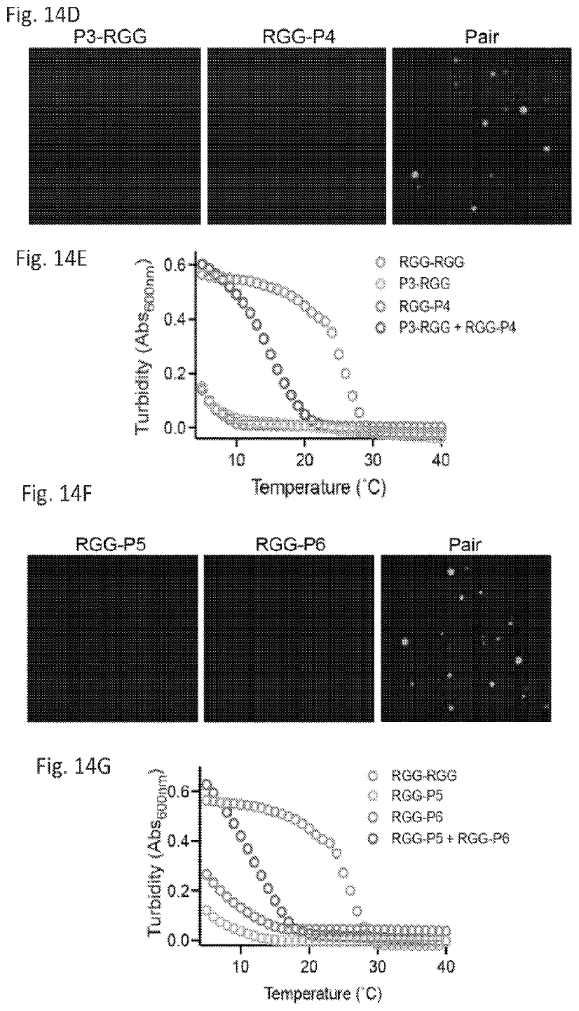

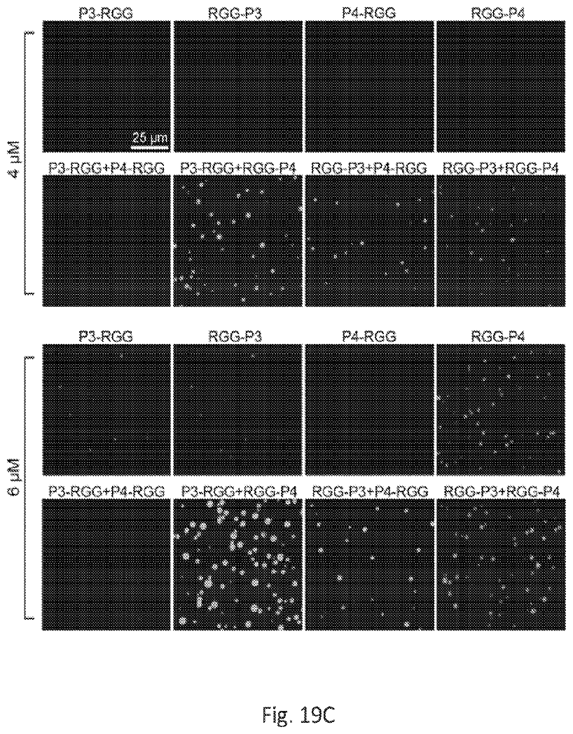

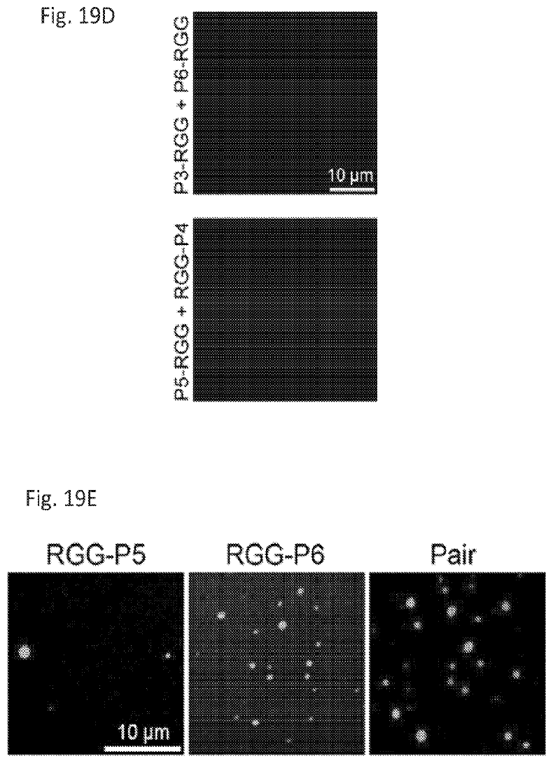

Modular, short protein helical coiled-coil bundles have been used to dimerize and target components in cells for nanoscale origami of protein structures and to generate switches via protein engineering. Results presented in Example 1 demonstrate the feasibility of recruiting exogenous proteins to an IDP condensate by tagging scaffold and client proteins with cognate SYNZIP (SZI and SZ2) coiled coils. Whether these coiled-coils could be used to stitch together higher order assemblies of the model IDR from LAF-1, the RGG domain, was investigated. A handful of coiled coils were tested, including the 48 aa SYNZIPs (Thompson, K. E. et al., ACS Synth Biol 2012, 1 (4), 118-29) and 33 aa Parallel Peptide Pairs (Lebar, T. et al., Nat Chem Biol 2020, 16 (5), 513-519). These coiled coils were cloned on the N- or C-terminus of the LAF-1 RGG domain and the phase separation behavior of individual and paired sets of proteins were characterized using microscopy and spectrophotometric turbidity assays (FIGS. 14A-14G). The LAF-1 RGG IDP sequence displays upper upper critical solution temperature (UCST) behavior: it is miscible at high temperatures and as the temperature is lowered, will condense at its phase boundary or “cloud point”, resulting in turbid solutions. Using microscopy at ambient temperature, it was possible to image condensate assembly either in brightfield or with fluorescent using a small amount of fluorescent RGG tracer (RGG-GFP-RGG) significantly below its Cₛₐₜ, such that it would not condense alone. As a validation of the biochemical conditions, RGG-RGG (constitutive dimer) formed robust condensates that could be visualized by fluorescence microscopy (FIG. 19A). Under these same conditions, the single RGG domain will not condense because of its much higher Cₛₐₜ.

Whether tagged-RGG remains miscible (OFF) in the monomer form, but could condense (ON) as a mixed heterodimer, was next tested using these assay conditions. A single RGG domain tagged with a folded SZI or SZ2 coiled coil was tested under physiological conditions at 4 μM protein concentration. Somewhat unexpectedly, SZ1-RGG and RGG-SZ2 both formed condensates on their own (FIG. 14B), suggesting a lower Cₛₐₜ than that of the untagged RGG alone. Upon mixing this cognate pair, the RGG domain dimerized and condensates grew larger. Additionally, a shift in critical temperature was observed in the turbidity assay (FIG. 14C). The lowered Cₛₐₜ of SYNZIP-tagged single RGG constructs may be explained by chain collapse due to reciprocal charges between the RGG domains (positively charged) and the coiled-coil tag (negatively charged). The RGG domain is enriched with positively charged arginine residues across its sequences which is involved in pi-pi contacts and contributes to its phase separation. Electrostatic interactions between the SYNZIPs and the RGG domain could result in partial chain collapse and several studies have demonstrated chain collapse is an important feature in the phase separation of disordered proteins: single chain collapse theory has been used to predict phase boundaries of disordered proteins. It is plausible that by introducing a motif with reciprocal charge, increased chain collapse was promoted, leading to a lower overall Cₛₐₜ and greater propensity to phase separate in the conditions used in the assay.

Because individual RGG domains tagged to SZI with SZ2 formed condensates on their own in the OFF state of these assay conditions, additional coiled-coil pairs were characterized. LAF-1 RGG domains tagged with different parallel coiled coils (P3 or P4) at either terminus do not condense at physiological conditions at 4 μM protein concentration (FIG. 14D, FIG. 19B). Yet, when paired, they form micron size condensates within minutes of mixing protein solutions. By increasing protein concentration, the phase boundary for RGG-P4 alone was identified, yet P3-RGG remained miscible, suggesting the monomers indeed have a higher Cₛₐₜ than the dimer (FIG. 19B). Upon varying the position of the P3 and P4 tags on the RGG termini, no coacervation of any of the monomer variants was observed. Of the mixed pairs at the same molar concentrations, P3-RGG+RGG-P4, RGG-P3+P4-RGG, and RGG-P3+RGG-P4, displayed condensation (FIG. 19C). Importantly, this condensation is specific to heterodimerization, as mixtures of non-cognate pairs of tagged-RGG did not generate condensates (FIG. 19D). The lowest critical concentration appeared to be for P3-RGG+RGG-P4 as it formed the largest condensates. The mixed pair of P3-RGG+P4-RGG did not form condensates, suggesting that N-terminal tagging may have an effect on either dimerization or RGG condensation. Notably, RGG contains an important 10 aa patch, residues 21-30, that promote condensation (Schuster, B. S. et al., Proc Natl Acad Sci USA 2020, 117 (21), 11421-11431). Turbidity measurements on P3-RGG and RGG-P4 support the microscopy and suggest that they are an excellent pair to control RGG valency and Cₛₐₜ (FIG. 14E). Phase separation of RGG tagged with a different set of parallel coiled coils, P5 and P6, was also tested (FIGS. 14F-14G). At 4 μM protein concentration, pH 8.5, and physiological salt, the P5-RGG and RGG-P6 monomers showed no visible condensation in the OFF state (FIG. 14F). The monomers also showed very little turbidity, even at lower temperatures, and appeared to heterodimerize nicely, creating a large increase in cloud point for the mixed pair, features that suggest they might be an ideal pair for controlling RGG valency. However, at pH 6.8 the same concentration of individual monomers condense on their own (FIG. 19E). Notably, the P3 and P4-tagged RGG monomers do not exhibit such pH sensitivity. Taken together, these data indicate that it is feasible to increase valency via protein interaction tags and that the type and position of the coiled-coil influences the level of miscibility in the OFF (monomer) state and efficacy of condensates in the ON state (pair, heterodimer). Additionally, these data indicate that the P3 and P4 heterodimers are quite compatible with dimerization and condensation of the LAF-1 RGG IDP and may be broadly useful for generating higher order disordered polymers.

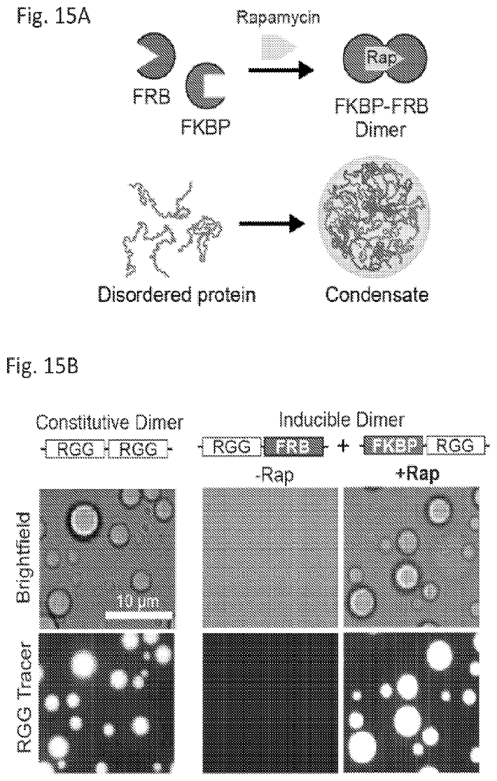

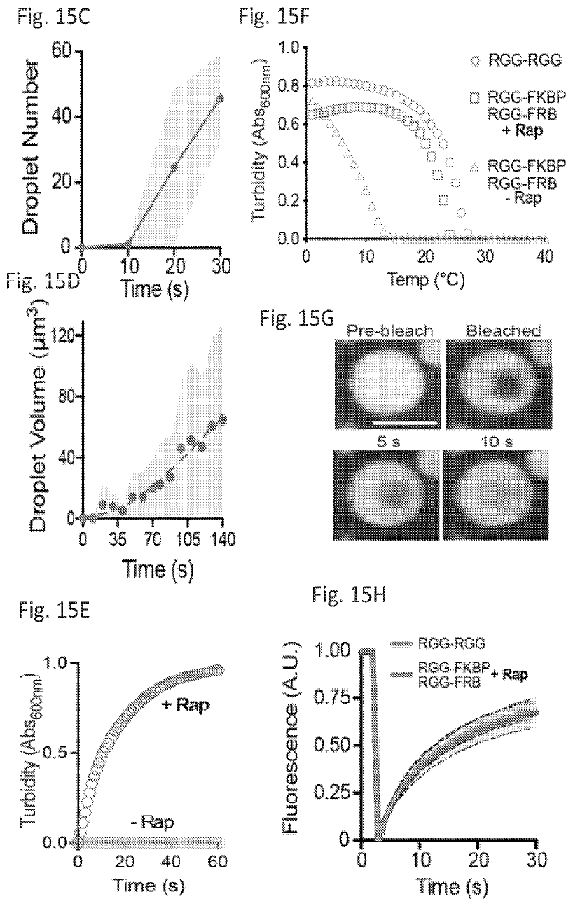

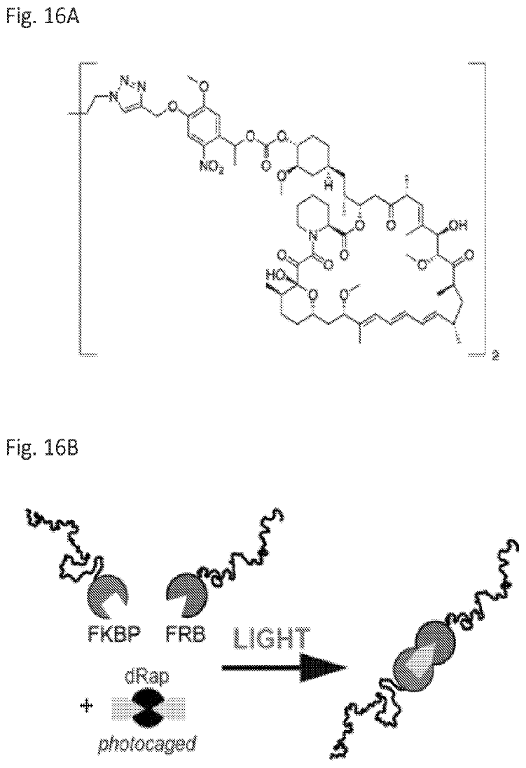

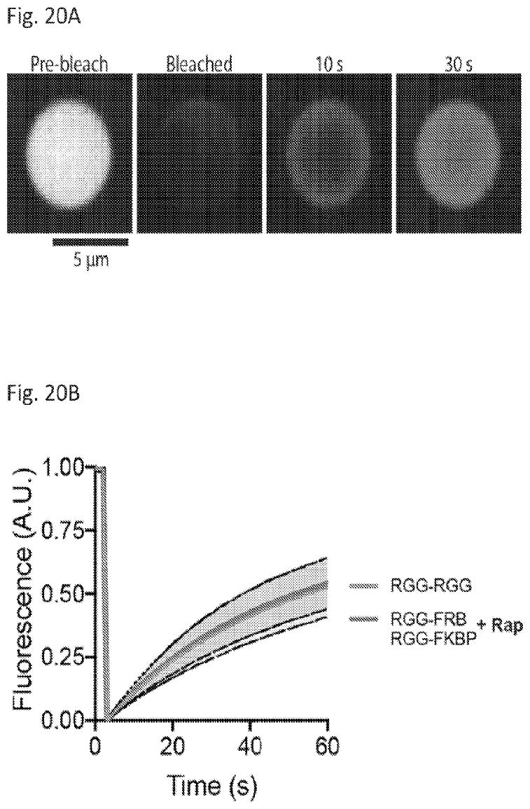

Upon validation of LAF-1 RGG heterodimerization as a means to increase polymer valency and self-assembly into condensates, whether coacervation with temporal precision could be induced by adding a small molecule was tested. Chemically responsive FRB and FKBP tags were fused to the RGG domains (FIGS. 15A-15C). FRB and FKBP domains form a ternary complex with rapamycin (Rap) and thus, when linked to RGG domains, Rap induces formation of a divalent RGG polymer (FIG. 15A). Because folded FRB and FKBP domains are larger than the coiled coils, they more significantly alter the order-to-disorder ratio of the polypeptide and increase the Cₛₐₜ. As a result, a slightly higher protein concentration was needed to visualize condensation of the dimerized state. A covalently linked RGG-RGG control dimer robustly phase separates into liquid droplets at ambient temperature and 10 μM concentration, consistent with previous observations. Monomeric RGG domains fused to FRB and FKBP tags are miscible in the same aqueous solution (FIG. 15B). Addition of Rap triggers rapid and robust phase separation to form micron scale condensates that enrich with the RGG-GFP-RGG tracer (FIGS. 15B-15D). Turbidity was measured over time in spectrophotometric turbidity assays after addition of Rap to quantify kinetics of the heterodimer assembly (FIG. 15E). Reactions lacking Rap become turbid, consistent with in vitro microscopy. Addition of Rap results in rapid solution turbidity with a t_{1/2} of ˜10 seconds (FIG. 15E), demonstrating efficient system responsiveness resulting from multimerization of RGG domains. Turbidity assays were next utilized to compare UCST behavior of Rap induced RGG dimers to the control RGG-RGG (FIG. 15F). Monomeric RGG domains fused to FRB or FKBP in the absence of Rap have critical temperatures similar to single RGG domains and dimerization in the presence of Rap results in turbidity curves similar to the RGG-RGG control. Finally, the liquidity of these condensates was assayed using fluorescence recovery after photobleaching (FRAP) to measure the dynamics of a fluorescent tracer localized to the condensed phase. Fluorescence of the RGG-GFP-RGG tracer in control condensates and those formed by Rap-mediated dimers rapidly recovers after photobleaching and show very similar recovery kinetics both via internal diffusion of fluorescent molecules as well as diffusion from outside condensates (FIGS. 15G, 15H, 20A, 20B). Taken together, these data demonstrate a strategy for chemogenic heterodimerization of IDP scaffolds to increase valency and induce formation of liquid-like condensates.

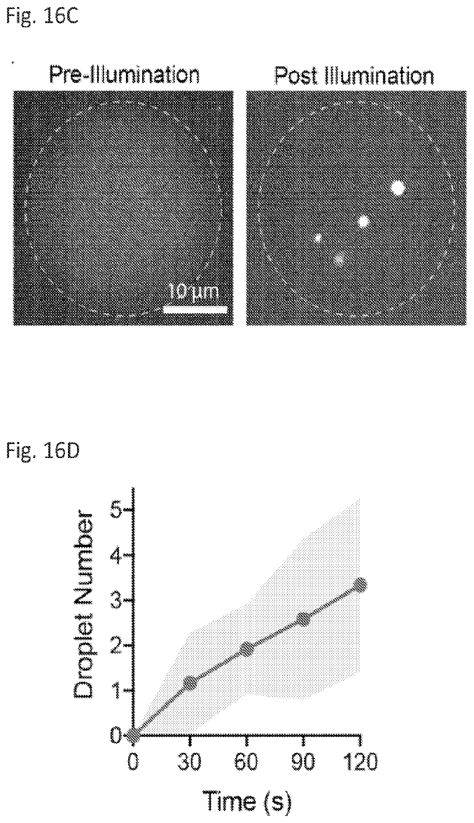

A useful feature of the FRB and FKBP domains is that their dimerization can also be optically regulated. A challenge of optogenetic dimerization domains is that their association requires sustained illumination, and many of these systems are difficult to reconstitute in vitro. In order to trigger irreversible condensation in vitro using only seconds of illumination, a photocaged version of rapamycin (dRap) in which a cage occludes the FRB binding sites was utilized, thereby preventing FRB-FKBP dimerization in the dark state (FIGS. 16A-16B). To achieve sufficient levels of illumination and mimic a cellular context, RGG-FRB and RGG-FKBP proteins were encapsulated with dRap in cell-size water-in-oil emulsions. This approach confines the reaction to picoliter volumes which can be entirely illuminated on a microscope. Prior to illumination, evenly dispersed signal from the RGG-GFP-RGG tracer was observed, indicating that no LLPS had occurred (FIG. 16C). Upon exposure to 405 nm light, rapid formation of protein droplets was observed, visible within tens of seconds (FIGS. 16C-16D). These data demonstrate a robust method for control of RGG domain valency in real-time to drive stable protein condensation, responsive to both small molecule and optical triggers.

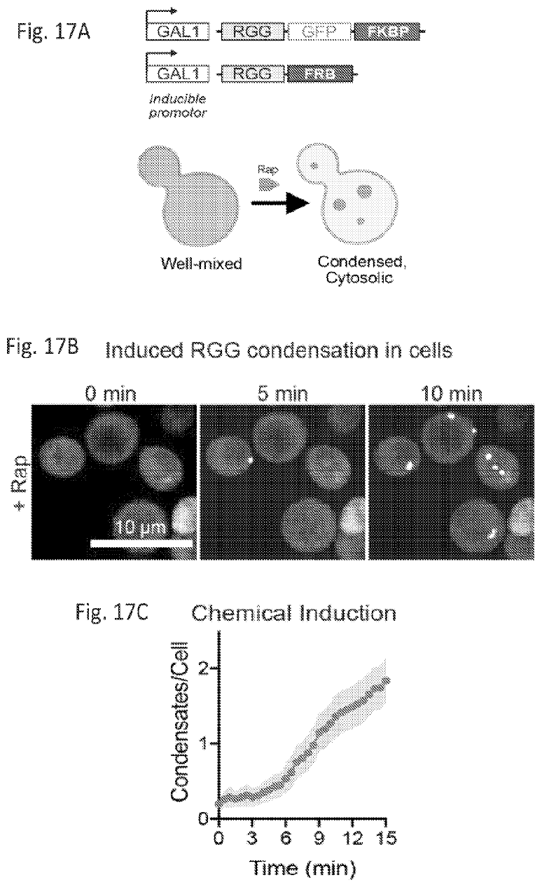

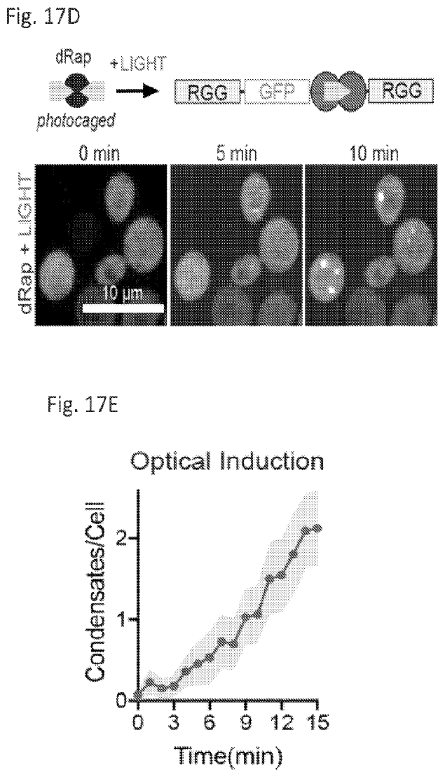

To demonstrate that this inducible RGG dimerization and condensation system can be extended to living systems, the monomer scaffold was encoded in a model single-cell organism. Budding yeast, S. cerevisiae, is a well-established system for studying aggregate formation and LLPS in vivo (FIGS. 17A-17E). Sequences encoding single RGG domains fused to FRB and FKBP were genomically integrated under the control of an inducible yeast GAL1 promoter. A GFP tag was included on one of the constructs (pGAL1-RGG-GFP-FKBP) in order to visualize condensate formation (FIG. 17A). Expression of these single RGG constructs was induced via galactose, and only diffuse GFP signal was observed, indicating that without dimerizer, they do not undergo LLPS (FIG. 17B). Upon addition of Rap, fluorescent puncta appeared. Condensates could be initially observed within tens of seconds and formed micron-size structures on the order of minutes, increasing in both number and size over time (FIGS. 17C, 21A). Next, whether the assembly of stable membraneless organelles could be induced using a single, short period of light exposure was tested. Using the same strain and encoded constructs as above, the photocaged dRap was added to the cells in media. Upon a 10 second pulse of 405 nm light, round puncta appeared and grew in number over time (FIGS. 14D-17E). Notably, in response to Rap addition or dRap optical uncaging, condensates also grew in size (FIGS. 17E, 21B). Further, the kinetics of droplet formation was determined to be very similar between both strategies above (FIG. 21C). In contrast to available optical LLPS systems in vitro, this strategy requires only a single short pulse of light to induce dimerization of a disordered sequence and cause protein coacervation.

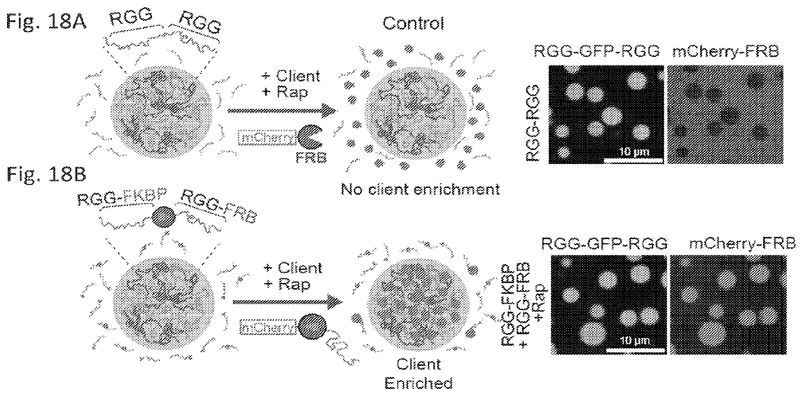

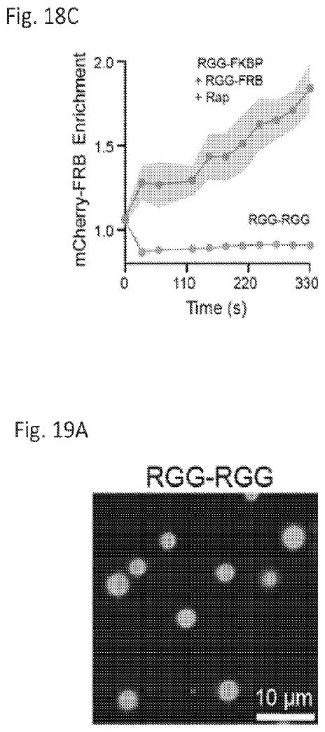

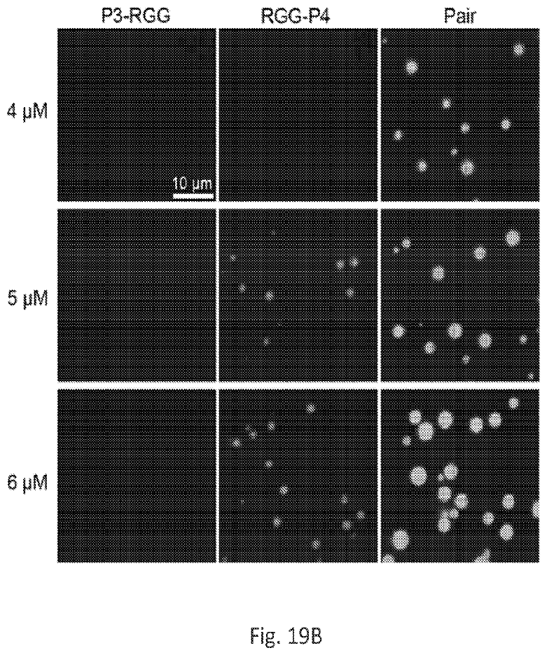

Next, whether chemogenic dimerization could be utilized to control recruitment of clients or cargo to the condensed phase, mimicking enzyme partitioning to membraneless organelles, was tested. To test the specificity and temporal kinetics of client recruitment, a model cargo, mCherry-FRB, was used. Enrichment of the model cargo was imaged in condensates formed from either RGG-RGG or heterodimers of RGG-FKBP+RGG-FRB in the presence of Rap (FIGS. 18A-18B). The cargo does not enrich in control RGG-RGG condensates, whereas it is recruited to and enriched in condensates of dimerized RGG-FKBP+RGG-FRB (FIG. 18C). This suggests selective client recruitment to the condensed phase dependent on dimerizing to RGG-FKBP. Overall, these data demonstrate a proof-of-concept for rapid and stable multimerization to form condensates both in vitro and in cell culture and provide new avenues for temporally regulating condensates assembly and composition, useful features for cellular engineering.

Characterizing the behavior of disordered protein sequences that self-assemble into condensed phases is important for understanding the biology of membraneless organelles and in bioengineering to generate gel-like and other materials for a range of therapeutic and industrial applications. The valency of an IDR was previously shown to determine critical concentrations for phase separation in vitro, similar to previous work with multivalent folded domains. Although proteolytic cleavage has been used to reduce IDR valency, the converse (i.e., building-up multivalency of an IDR sequence to regulate protein condensation in a predictable manner) was investigated herein. Noncovalent dimerization of the LAF-1 RGG domain was systematically engineered via coiled coil motifs, and real-time control of IDR condensation and client recruitment using folded optochemical dimerization domains was further demonstrated. Based on these initial findings, it is contemplated herein that one could increase valency to trimeric and tetramer IDRs, to alter Cₛₐₜ or promote temperature resistant LLPS in cells, and also to fine-tune the desired physicochemical properties of the condensed phase.

There may be several reasons why terminal position of the coiled coil can influence the LLPS of the monomer IDR. First, the LAF-1 RGG domain harbors a well-conserved and critical motif at its N-terminal end, aa 21-30 and thus tagging may interfere clustering of this motif. Also, the RGG domain has well-dispersed charge along the polypeptide chain, but it also has slightly more positively charged at its C-terminal end, which may interact with the net negative charge of the helical coils whose isoelectric points are between 4 and 5, and thus affect phase separation by altering the kinetics of chain collapse. Future study using positively charged coiled coils would be of interest. Despite the size and structured nature of FKBP and FRB tags, these tagged constructs formed condensates that behaved similarly to dimeric RGG controls, suggesting these tagging with folded domains did not significantly alter the Cₛₐₜ. This is likely due to use of a 168 aa IDR, and the ratio of IDR to folded protein molecular mass certainly affect saturation concentration for LLPS.

Multivalency has been used previously as a useful strategy for driving self-assembly and coacervation for both structured and disordered proteins. The present study provides paradigms for sequential multimerization of IDRs for applications in materials science, synthetic biology and cellular engineering. The short coiled-coils are readily knocked in, using CRISPR, to native gene loci without disrupting endogenous protein function as shown herein (Example 1). Further, it is contemplated herein that more complex assemblies using multiple sets of coiled-coils in ‘protein origami’ could be used to build novel architectures that potentially nucleate condensation.

### Example 4

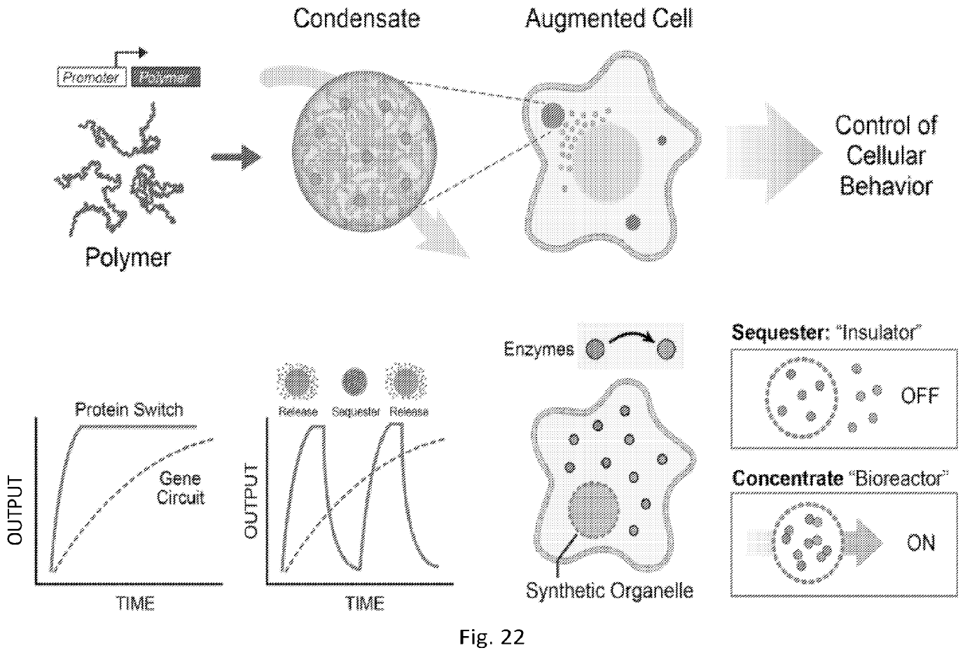

FIG. 22: Schematic of augmenting cells with synthetic organelles that function as fast-acting protein switches. Platform has potential to be run in two functional modes: insulator, or bioreactor.

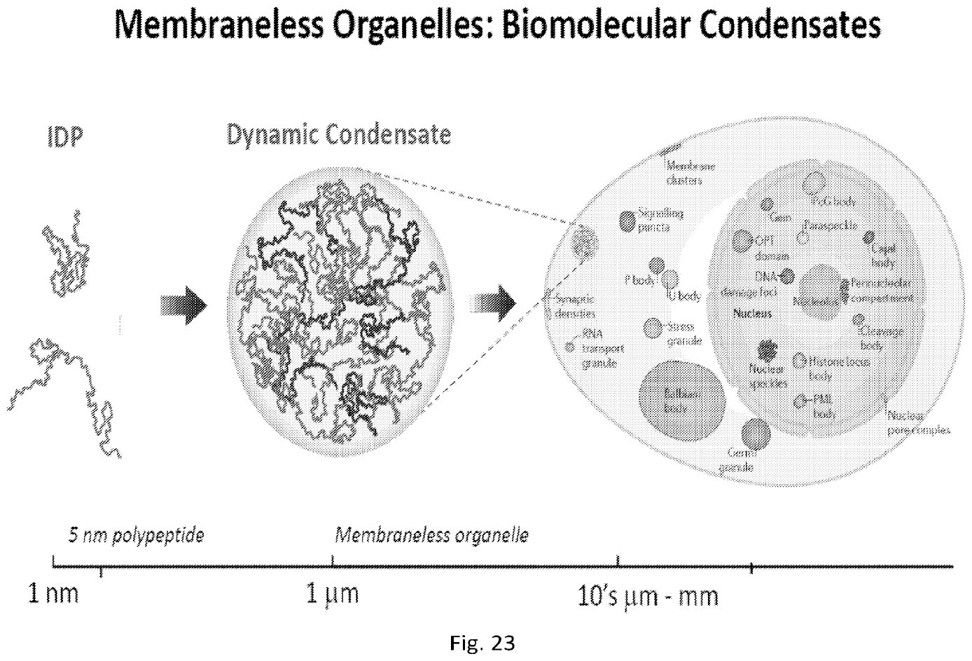

FIG. 23: Biomolecular condensates as membraneless organelles in the cell. They form from self-assembly of disordered proteins and RNA into mesoscale subcompartments.

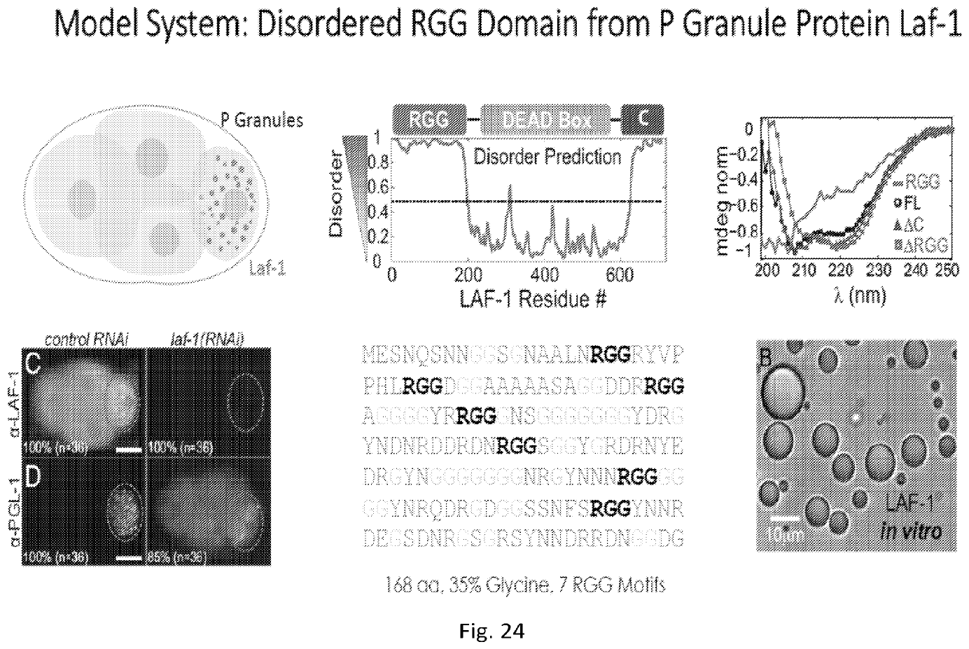

FIG. 24: Disordered RGG domain from P granule protein Laf-1 is necessary and sufficient for condensate formation in vitro.

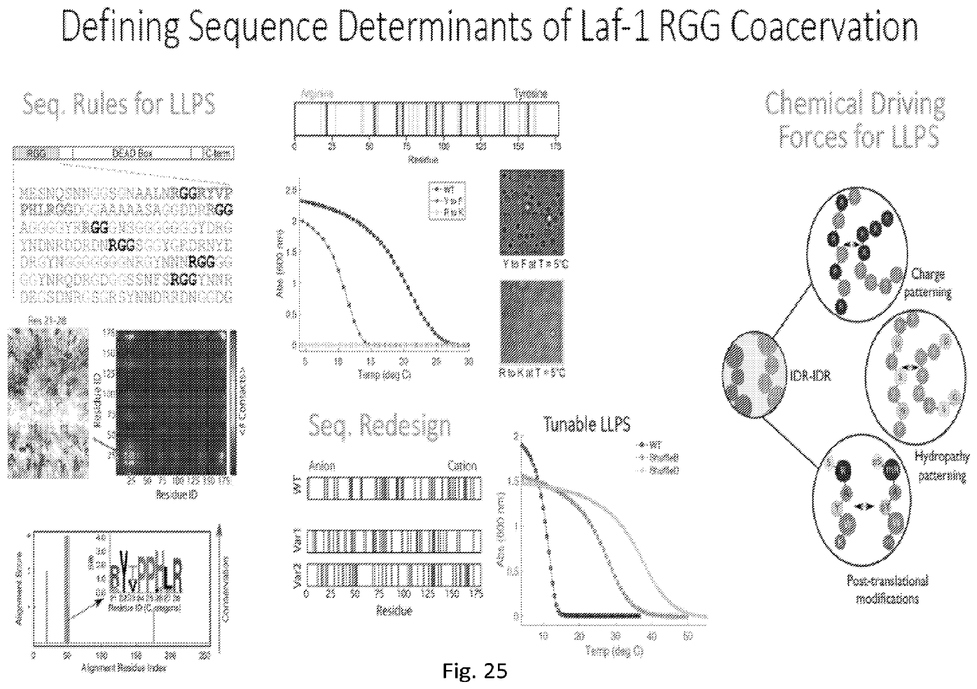

FIG. 25: Summary of sequence determinants of Laf-1 RGG LLPS.

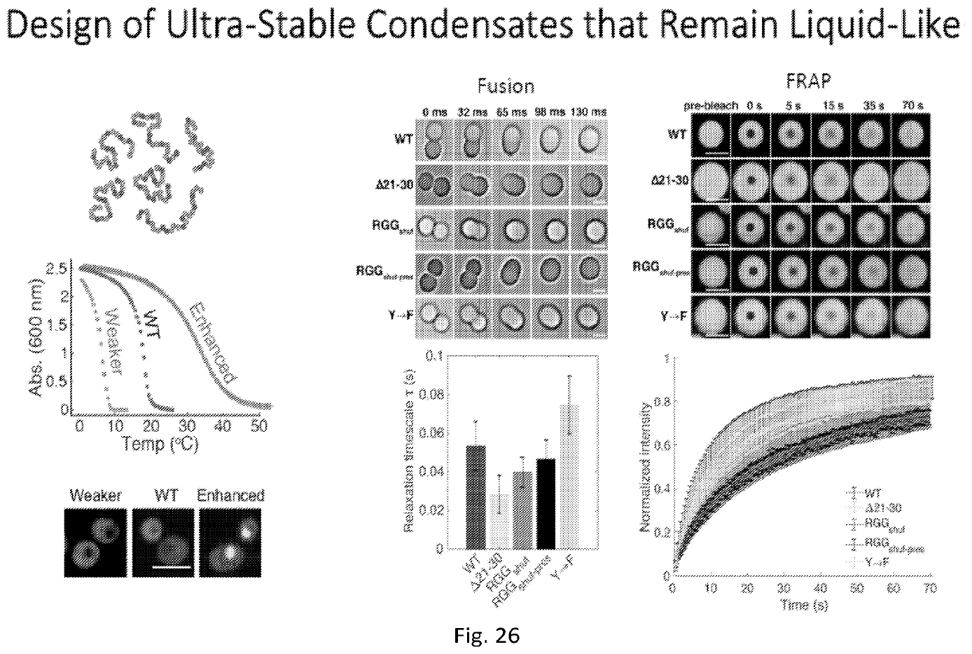

FIG. 26: Mutation of Laf-1 RGG to raise or lower its critical concentration for LLPS does not alter the liquid-like nature of condensates in vitro.

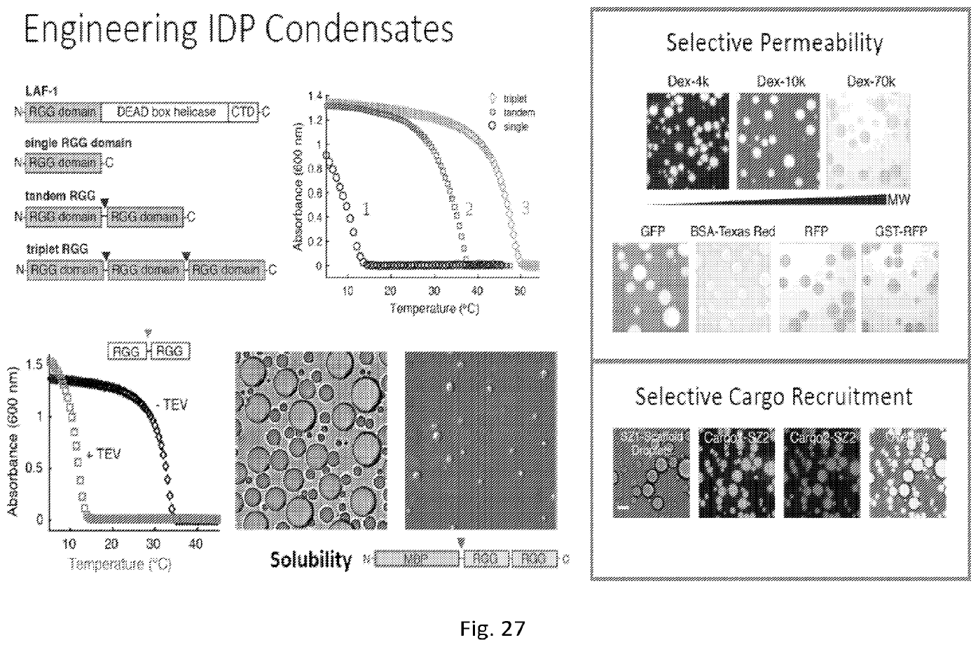

FIG. 27: Demonstration of utility of Laf-1 RGG to form engineered coacervates in vitro.

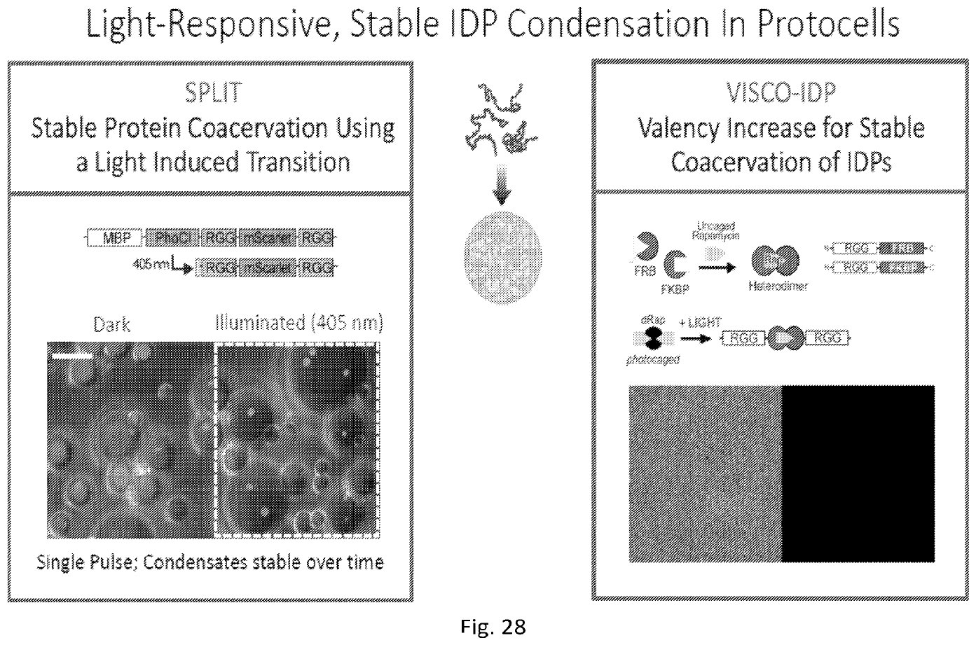

FIG. 28: Embedding of optical regulatory handles into Laf-1 RGG sequence to control its LLPS using light.

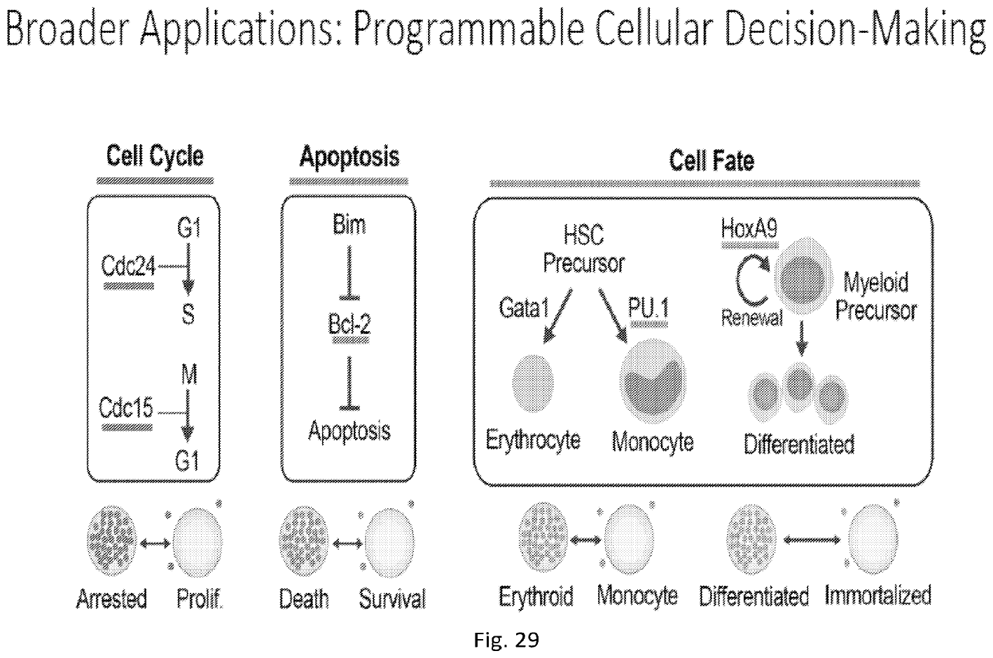

FIG. 29: Schematic summary of applications of synthetic organelle platform to sequester pathway components in key subcellular processes, including cell cycle control system, cell death via apoptosis and cell fate control.

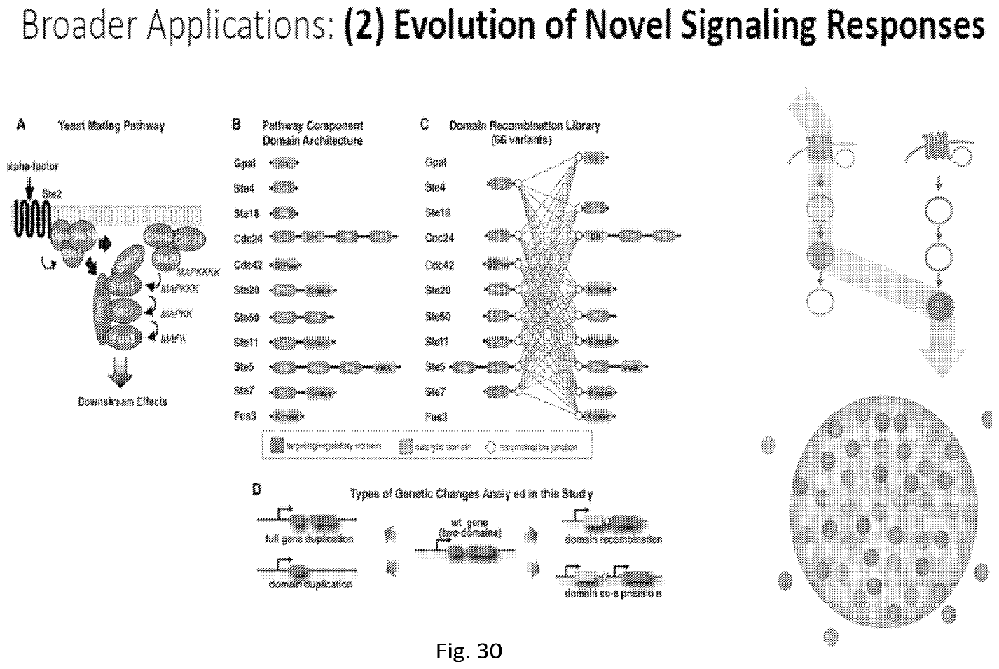

FIG. 30: Schematic summary of application of synthetic organelle as a hub to rewire signal transmission.

### Enumerated Embodiments

The following enumerated embodiments are provided, the numbering of which is not to be construed as designating levels of importance.

Embodiment 1 provides a synthetic organelle comprising a first nucleic acid sequence encoding an intrinsically disordered protein (IDP) scaffold comprising three arginine/glycine-rich (RGG) domains, a first high-affinity coiled-coil (CC) tag, and a first promoter; and a second nucleic acid sequence encoding a client protein, a second CC tag, and a second promoter.

Embodiment 2 provides the synthetic organelle of embodiment 1, wherein the first or second CC tag is selected from the group consisting of SZ1, SZ2, TsCC(A), and TsCC(B).

Embodiment 3 provides the synthetic organelle of any of the preceding embodiments, wherein the first CC tag is TsCC(A) and the second CC tag is TsCC(B).

Embodiment 4 provides the synthetic organelle of embodiment 3, wherein when TsCC(A) interacts with TsCC(B), the client protein is sequestered in the synthetic organelle, and wherein when temperature is raised, the client protein is released from the synthetic organelle.

Embodiment 5 provides the synthetic organelle of any of the preceding embodiments, wherein the CC tag is encoded by the nucleotide sequence of any of SEQ ID NOs: 7, 8, 9, or 10; or comprises the amino acid sequence of any of SEQ ID NOs: 17, 18, 19, or 20.

Embodiment 6 provides the synthetic organelle of any of the preceding embodiments, wherein the RGG domains are RGG1, RGG2, and RGG3 from the Caenorhabditis elegans LAF-1 protein.

Embodiment 7 provides the synthetic organelle of embodiment 6, wherein the RGG domains are encoded by the nucleotide sequence of any of SEQ ID NOs: 1-6, or comprise the amino acid sequence of SEQ ID NO: 16.

Embodiment 8 provides the synthetic organelle of any of the preceding embodiments, wherein the client protein is an endogenous enzyme.

Embodiment 9 provides the synthetic organelle of any of the preceding embodiments, wherein the client protein regulates a cellular function.

Embodiment 10 provides the synthetic organelle of any of the preceding embodiments, wherein the first and/or second nucleic acid encodes a photocleavable protein or a fluorescent tag.

Embodiment 11 provides the synthetic organelle of claim 10, wherein the photocleavable protein or fluorescent tag is selected from the group consisting of PhoCl, PhoCl 2F, EGFP, mScarlet, iRFP and mCherry.

Embodiment 12 provides the synthetic organelle of claim 11, wherein when the synthetic organelle is exposed to light, the photocleavable protein is cleaved and the client is released.

Embodiment 13 provides the synthetic organelle of claim 11, wherein the photocleavable protein or fluorescent tag is encoded by a nucleotide sequence of any of SEQ ID NOs: 11, 12, or 13.

Embodiment 14 provides the synthetic organelle of any of the preceding embodiments wherein the first and/or second nucleic acid encodes a drug-induced dimerization domain.

Embodiment 15 provides the synthetic organelle of embodiment 14, wherein the drug-induced dimerization domain is FRB or FKBP.

Embodiment 16 provides the synthetic organelle of embodiment 14, wherein the drug-induced dimerization domain is encoded by the nucleotide sequence of any of SEQ ID NOs: 14 or 15: or comprises the amino acid sequence of any of SEQ ID NOs: 24 or 25.

Embodiment 17 provides the synthetic organelle of any of the preceding embodiments, wherein the first promoter is an inducible promoter and the second promoter is a constitutive promoter.

Embodiment 18 provides the synthetic organelle of any of the preceding embodiments, wherein the second promoter is an endogenous promoter.

Embodiment 19 provides a synthetic organelle comprising a first nucleic acid encoding an intrinsically disordered protein (IDP) scaffold comprising three arginine/glycine-rich (RGG) domains, a first high-affinity coiled-coil (CC) tag, and a first promoter; and
a second nucleic acid encoding a client protein, a second CC tag, and a second promoter, wherein the second promoter is an endogenous promoter and the second CC tag tags an endogenous genomic loci.

Embodiment 20 provides a cell comprising the synthetic organelle of any of the preceding embodiments.

Embodiment 21 provides the cell of embodiment 20, wherein the cell is a mammalian cell.

Embodiment 22 provides the cell of embodiment 20, wherein the cell is a human cell.

Embodiment 23 provides a lentiviral vector comprising the synthetic organelle of any of the preceding claims.

Embodiment 24 provides a lentiviral vector comprising a nucleotide sequence encoding a promoter, and an IDP scaffold comprising three RGG domains and a CC tag.

Embodiment 25 provides the lentiviral vector of any of the preceding embodiments, wherein the CC tag is Syn ZIP1 or TsCC(A).

Embodiment 26 provides the lentiviral vector of any of the preceding embodiments, wherein the promoter is a constitutive CMV viral promoter or a Tet-ON-3G drug-inducible promoter.

Embodiment 27 provides the lentiviral vector of embodiment 24, wherein the promoter is a tetracycline responsive TRE3G promoter.

Embodiment 28 provides a cell comprising the lentiviral vector of any of the preceding embodiments.

Embodiment 29 provides the cell of embodiment 21, wherein the cell further comprises a nucleic acid encoding a client protein, a second CC tag, and a second promoter.

Embodiment 30 provides the cell of embodiment 21, wherein the cell further comprises a nucleic acid encoding a rtTA transactivator.

Embodiment 31 provides the cell of embodiment 21, wherein the cell is a mammalian cell.

Embodiment 32 provides the cell of embodiment 21, wherein the cell is a human cell.

Embodiment 33 provides the cell of embodiment 21, wherein the cell further comprises a packaging plasmid and/or an envelope plasmid.

Embodiment 34 provides a method of controlling at least one cellular process in a cell, the method comprising administering to the cell: a first nucleic acid sequence encoding an IDP scaffold comprising three RGG domains, a first high-affinity coiled-coil (CC) tag, and a first promoter; and a second nucleic acid sequence encoding a client protein, a second CC tag, and a second promoter, wherein the client protein is a cellular decision making protein, wherein when the scaffold is expressed, a sequesterable construct is formed and at least one cellular process is controlled.

Embodiment 35 provides a method of controlling at least one cellular process in a mammalian cell, the method comprising administering to the cell: a first nucleic acid sequence encoding an IDP scaffold comprising three RGG domains, a first high-affinity coiled-coil (CC) tag, and a first promoter; and a second nucleic acid sequence encoding a client protein and a second CC tag, wherein the CC tag is inserted into the genome of the mammalian cell in a region encoding a cellular decision making protein, wherein when the scaffold is expressed, a sequesterable construct is formed and at least one cellular process is controlled.

Embodiment 36 provides the method of any of the preceding embodiments, wherein the first or second CC tag is selected from the group consisting of SZI, SZ2, TsCC(A), and TsCC(B).

Embodiment 37 provides the method of any of the preceding embodiments, wherein the first CC tag is TsCC(A) and the second CC tag is TsCC(B).

Embodiment 38 provides the method of any of the preceding embodiments, wherein when TsCC(A) interacts with TsCC(B), the client protein is sequestered, and wherein when temperature is raised, the client protein is released.

Embodiment 39 provides the method of any of the preceding embodiments, wherein the CC tag is encoded by the nucleotide sequence of any of SEQ ID NOs: 7, 8, 9, or 10; or comprises the amino acid sequence of any of SEQ ID NOs: 17, 18, 19, or 20.

Embodiment 40 provides the method of any of the preceding embodiments, wherein the RGG domains are RGG1, RGG2, and RGG3 from the Caenorhabditis elegans LAF-1 protein.

Embodiment 41 provides the method of any of the preceding embodiments, wherein the RGG domains are encoded by the nucleotide sequence of any of SEQ ID NOs: 1-6, or comprise the amino acid sequence of SEQ ID NO: 16.

Embodiment 42 provides the method of any of the preceding embodiments, wherein the first and/or second nucleic acid encodes a photocleavable protein or a fluorescent tag.

Embodiment 43 provides the method of embodiment 42, wherein the photocleavable protein or fluorescent tag is selected from the group consisting of PhoCl, PhoCl 2F, EGFP, mScarlet, iRFP and mCherry.

Embodiment 44 provides the method of embodiment 42, wherein when the cell is exposed to light, the photocleavable protein is cleaved and the client is released.

Embodiment 45 provides the method of embodiment 42, wherein the photocleavable protein or fluorescent tag is encoded by a nucleotide sequence of any of SEQ ID NOs: 11, 12, or 13.

Embodiment 46 provides the method of any of the preceding embodiments, wherein the first and/or second nucleic acid encodes a drug-induced dimerization domain.

Embodiment 47 provides the method of embodiment 46, wherein the drug-induced dimerization domain is FRB or FKBP.

Embodiment 48 provides the method of embodiment 46, wherein the drug-induced dimerization domain is encoded by the nucleotide sequence of any of SEQ ID NOs: 14 or 15; or comprises the amino acid sequence of any of SEQ ID NOs: 24 or 25.

Embodiment 49 provides the method of any of the preceding embodiments, wherein the first promoter is an inducible promoter and the second promoter is a constitutive promoter.

Embodiment 50 provides the method of any of the preceding embodiments, wherein the second promoter is an endogenous promoter.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A synthetic organelle comprising
a first nucleic acid sequence encoding an intrinsically disordered protein (IDP) scaffold comprising three arginine/glycine-rich (RGG) domains, a first high-affinity coiled-coil (CC) tag, and a first promoter; and
a second nucleic acid sequence encoding a client protein, a second CC tag, and a second promoter.

2. The synthetic organelle of claim 1, wherein the first or second CC tag is selected from the group consisting of SZI, SZ2, TsCC(A), and TsCC(B).

3. The synthetic organelle of claim 1, wherein the first CC tag is TsCC(A) and the second CC tag is TsCC(B).

4. The synthetic organelle of claim 3, wherein when TsCC(A) interacts with TsCC(B), the client protein is sequestered in the synthetic organelle, and wherein when temperature is raised, the client protein is released from the synthetic organelle.

5. The synthetic organelle of claim 1, wherein the CC tag is encoded by the nucleotide sequence of any of SEQ ID NOs: 7, 8, 9, or 10; or comprises the amino acid sequence of any of SEQ ID NOs: 17, 18, 19, or 20.

6. The synthetic organelle of claim 1, wherein the RGG domains are RGG1, RGG2, and RGG3 from the Caenorhabditis elegans LAF-1 protein.

7. The synthetic organelle of claim 6, wherein the RGG domains are encoded by the nucleotide sequence of any of SEQ ID NOs: 1-6, or comprise the amino acid sequence of SEQ ID NO: 16.

8. The synthetic organelle of claim 1, wherein the client protein is an endogenous enzyme.

9. The synthetic organelle of claim 1, wherein the client protein regulates a cellular function.

10. The synthetic organelle of claim 1, wherein the first and/or second nucleic acid encodes a photocleavable protein or a fluorescent tag.

11. The synthetic organelle of claim 10, wherein the photocleavable protein or fluorescent tag is selected from the group consisting of PhoCl, PhoCI 2F, EGFP, mScarlet, iRFP and mCherry.

12. The synthetic organelle of claim 11, wherein when the synthetic organelle is exposed to light, the photocleavable protein is cleaved and the client is released.

13. The synthetic organelle of claim 11, wherein the photocleavable protein or fluorescent tag is encoded by a nucleotide sequence of any of SEQ ID NOs: 11, 12, or 13.

14. The synthetic organelle of claim 1, wherein the first and/or second nucleic acid encodes a drug-induced dimerization domain.

15. The synthetic organelle of claim 14, wherein the drug-induced dimerization domain is FRB or FKBP.

16. The synthetic organelle of claim 14, wherein the drug-induced dimerization domain is encoded by the nucleotide sequence of any of SEQ ID NOs: 14 or 15; or comprises the amino acid sequence of any of SEQ ID NOs: 24 or 25.

17. The synthetic organelle of claim 1, wherein the first promoter is an inducible promoter and the second promoter is a constitutive promoter.

18. The synthetic organelle of claim 1, wherein the second promoter is an endogenous promoter.

19. A synthetic organelle comprising
a first nucleic acid encoding an intrinsically disordered protein (IDP) scaffold comprising three arginine/glycine-rich (RGG) domains, a first high-affinity coiled-coil (CC) tag, and a first promoter; and
a second nucleic acid encoding a client protein, a second CC tag, and a second promoter, wherein the second promoter is an endogenous promoter and the second CC tag tags an endogenous genomic loci.

20. A cell comprising the synthetic organelle of claim 1.

21. The cell of claim 20, wherein the cell is a mammalian cell.

22. The cell of claim 20, wherein the cell is a human cell.

23. A lentiviral vector comprising the synthetic organelle of claim 1.

24. A lentiviral vector comprising a nucleotide sequence encoding a promoter, and an IDP scaffold comprising three RGG domains and a CC tag.

25. The lentiviral vector of claim 1, wherein the CC tag is Syn ZIP1 or TsCC(A).

26. The lentiviral vector of claim 1, wherein the promoter is a constitutive CMV viral promoter or a Tet-ON-3G drug-inducible promoter.

27. The lentiviral vector of claim 24, wherein the promoter is a tetracycline responsive TRE3G promoter.

28. A cell comprising the lentiviral vector of claim 1.

29. The cell of claim 21, wherein the cell further comprises a nucleic acid encoding a client protein, a second CC tag, and a second promoter.

30. The cell of claim 21, wherein the cell further comprises a nucleic acid encoding a rtTA transactivator.

31. The cell of claim 21, wherein the cell is a mammalian cell.

32. The cell of claim 21, wherein the cell is a human cell.

33. The cell of claim 21, wherein the cell further comprises a packaging plasmid and/or an envelope plasmid.

34. A method of controlling at least one cellular process in a cell, the method comprising administering to the cell:
a first nucleic acid sequence encoding an IDP scaffold comprising three RGG domains, a first high-affinity coiled-coil (CC) tag, and a first promoter; and
a second nucleic acid sequence encoding a client protein, a second CC tag, and a second promoter, wherein the client protein is a cellular decision making protein,
wherein when the scaffold is expressed, a sequesterable construct is formed and at least one cellular process is controlled.

35. A method of controlling at least one cellular process in a mammalian cell, the method comprising administering to the cell:
a first nucleic acid sequence encoding an IDP scaffold comprising three RGG domains, a first high-affinity coiled-coil (CC) tag, and a first promoter; and
a second nucleic acid sequence encoding a client protein and a second CC tag, wherein the CC tag is inserted into the genome of the mammalian cell in a region encoding a cellular decision making protein, wherein when the scaffold is expressed, a sequesterable construct is formed and at least one cellular process is controlled.

36. The method of claim 34, wherein the first or second CC tag is selected from the group consisting of SZ1, SZ2, TsCC(A), and TsCC(B).

37. The method of claim 34, wherein the first CC tag is TsCC(A) and the second CC tag is TsCC(B).

38. The method of claim 34, wherein when TsCC(A) interacts with TsCC(B), the client protein is sequestered, and wherein when temperature is raised, the client protein is released.

39. The method of claim 34, wherein the CC tag is encoded by the nucleotide sequence of any of SEQ ID NOs: 7, 8, 9, or 10; or comprises the amino acid sequence of any of SEQ ID NOs: 17, 18, 19, or 20.

40. The method of claim 34, wherein the RGG domains are RGG1, RGG2, and RGG3 from the Caenorhabditis elegans LAF-1 protein.

41. The method of claim 34, wherein the RGG domains are encoded by the nucleotide sequence of any of SEQ ID NOs: 1-6, or comprise the amino acid sequence of SEQ ID NO: 16.

42. The method of claim 34, wherein the first and/or second nucleic acid encodes a photocleavable protein or a fluorescent tag.

43. The method of claim 42, wherein the photocleavable protein or fluorescent tag is selected from the group consisting of PhoCl, PhoCI 2F, EGFP, mScarlet, iRFP and mCherry.

44. The method of claim 42, wherein when the cell is exposed to light, the photocleavable protein is cleaved and the client is released.

45. The method of claim 42, wherein the photocleavable protein or fluorescent tag is encoded by a nucleotide sequence of any of SEQ ID NOs: 11, 12, or 13.

46. The method of claim 34, wherein the first and/or second nucleic acid encodes a drug-induced dimerization domain.

47. The method of claim 46, wherein the drug-induced dimerization domain is FRB or FKBP.

48. The method of claim 46, wherein the drug-induced dimerization domain is encoded by the nucleotide sequence of any of SEQ ID NOs: 14 or 15; or comprises the amino acid sequence of any of SEQ ID NOs: 24 or 25.

49. The method of claim 34, wherein the first promoter is an inducible promoter and the second promoter is a constitutive promoter.

50. The method of claim 34, wherein the second promoter is an endogenous promoter.


SEQUENCE LISTING (25 sequences)

SEQ ID NO: 1 - DNA, 501 bp, synthetic construct
  source 1..501: mol_type = other DNA
gaatcgaatcaatctaacaacggtggatcgggaaacgcagcattgaatcgtgggggccgttacgtaccaccacacttgagaggcggagatggtggtgccgccgctgctgcctcagccggaggcgacgacagacgcggtggggccgggggtggtggctatcgtagaggtggtggaaattcagggggcggcggcggaggaggctacgatcgtggatacaacgacaaccgggatgaccgtgacaatcggggcggctccggcggttatggccgggacagaaattacgaggatcgcggttataacgggggtggtgggggaggaggcaatcgcggctacaataataaccgcggtgggggtggaggcggctataatcggcaggaccgtggagacggcggttctagtaatttctcgcggggagggtataacaacagagatgagggctcagacaacagaggctcaggcagatcatacaacaacgatcggcgcgacaatggtggtgacggg

SEQ ID NO: 2 - DNA, 501 bp, synthetic construct
  source 1..501: mol_type = other DNA
gaatccaatcaatccaataacggtggctcgggtaatgccgctctgaatcgtggtggccgctatgtgccgccgcatctgcgtggtggcgatggtggtgcagcagcagctgcatctgctggcggtgatgaccgtcgcggcggtgcgggcggtggcggttatcgtcgcggcggtggcaatagtggtggcggtggcggtggcggttatgatcgtggttacaatgacaaccgtgatgaccgcgataaccgtggcggttccggcggttacggtcgtgatcgtaattatgaagaccgtggttacaacggcggtggcggtggcggtggcaatcgtggctataacaataaccgtggtggcggtggcggtggctacaatcgccaggatcgtggtgacggtggcagctctaacttttcgcgcggtggctataataaccgtgatgaaggcagcgacaaccgcggtagtggccgttcctacaataacgatcgtcgcgacaatggtggcgatggc

SEQ ID NO: 3 - DNA, 501 bp, synthetic construct
  source 1..501: mol_type = other DNA
gaatctaaccaatcaaacaatggcggctccggtaacgcggcgcttaacaggggcggtcgttacgtgcctccgcacctgagagggggcgacggaggtgcggctgctgccgcgagtgccggtggtgatgacaggcgtggtggtgcgggtggcgggggctatcgtcgtggaggcggtaattccgggggaggtggtggtgggggttacgacagagggtacaatgataaccgtgatgatcgtgacaacagaggcgggagtggcggttacggtagggatcgtaactatgaagacagaggctataacgggggtggtggtggcggtggcaatagaggttataataacaaccgtggcggcggtggaggcgggtataacagacaggatagaggggacggcggcagtagcaacttctcccgtggcggatataacaaccgtgacgaaggctctgacaataggggttctggtaggtcttacaataatgataggagagacaatggcggcgacggt

SEQ ID NO: 4 - DNA, 501 bp, synthetic construct; identical to SEQ ID NO: 1
  source 1..501: mol_type = other DNA

SEQ ID NO: 5 - DNA, 501 bp, synthetic construct
  source 1..501: mol_type = other DNA
gaatccaatcagtccaataatgggggcagcgggaacgcggccttgaatcgcggtgggcggtacgttcctcctcatttacgaggtggagatggaggcgctgcagccgctgcaagtgcaggaggtgacgatcgtagaggcggagctggaggcggaggttatagacggggaggtgggaactcaggaggcggcggtggtggagggtacgacagaggttataacgacaatcgagatgatcgggataaccgaggtgggagcgggggttacgggcgcgatcgcaattacgaggacaggggttataatggtgggggcggaggaggcggaaaccgggggtataacaataatcggggagggggaggtggagggtataaccgccaggaccgaggtgacggagggtcaagcaatttttcacgcgggggctacaacaaccgcgacgagggcagtgacaatcggggtagtggcaggagttacaacaatgatagaagggacaacggcggtgatggg

SEQ ID NO: 6 - DNA, 501 bp, synthetic construct
  source 1..501: mol_type = other DNA
gagtctaatcagagcaataacgggggtagtggcaatgccgcgctcaatcggggaggccgctacgtgcctcctcacctcagagggggcgatgggggcgccgctgccgctgcgagcgccggtggcgatgatcggcggggaggagcgggtggaggtggttatcgccgaggtggcgggaacagtgggggtggaggaggcggtggatatgatcgaggttacaacgacaatcgggacgatcgagataacagagggggttcaggtggatacggaagagacaggaattacgaggacagaggatacaatggcggaggcggtgggggaggtaatagaggatacaacaataatcgaggaggcggggggggagggtacaatcgccaagaccgcggtgatggaggaagttctaactttagcagaggtggatacaacaatagggatgaaggctcagacaacagaggttctggtcgaagctacaataacgacagacgcgacaacggtggcgatggg

SEQ ID NO: 7 - DNA, 144 bp, synthetic construct
  source 1..144: mol_type = other DNA
atgaacttggtagcccagcttgagaacgaagttgcgagtctggaaaacgaaaacgagacgttgaagaaaaagaacttgcacaagaaagatttgattgcctacttggaaaaggaaattgccaatctgcgtaaaaagattgaggaa

SEQ ID NO: 8 - DNA, 153 bp, synthetic construct
  source 1..153: mol_type = other DNA
atggcgcgtaacgcatatcttcgtaagaaaattgcacggctgaaaaaggacaatctacagttggaacgcgacgagcagaacctcgagaagatcattgctaatctgcgcgacgagattgctcgcttagaaaacgaagtcgcatcacatgagcag

SEQ ID NO: 9 - DNA, 918 bp, synthetic construct
  source 1..918: mol_type = other DNA
gtggaagtggctgaagaagtgaaggccgtctccgccgcgctgtccgaacgcatcacccagctggcgacagaactgaatgacaaggcggtccgggctgcagaacgccgggttgcggaagtcacgcgtgctgccggtgaacagaccgcacaggcagagcgggagctggccgacgccgcgcagacagtcgacgacctggaagaaaaactggttgaactgcaggacagatatgacagtttgacgctggcgctggagtcagaacgttcactgcgtcagcagcatgatgtggagatggcccagctgaaagagcgtcttgcggccgctgaagagaatacccgtcagcgacgtgaacggtatcaggagcagaagacagtgctgcaggatgcgcttaatgcggagcaggcacagcacaaaaacacgcgggaagacctgcagaaacgactggagcaaatttctgtcgaagctaatgcgcgtacagaagaactgaagtctgaacgcgataaagtcaatactttccttacccgccttgaatcgcaggaaaatgcgctggcctcacgtcgtcagcagcatctggccacccgcgaaacgctgcagcaacgcctcgagcaggccatcgctgacacgcaggcgcgcgccggtgagattgcacttgaacgtgacagagtcagcagcctcaccgcaaggctggaatcgcaggaaaaggcctcctcggagcaactggtgcgtatgggcagtgaaatagccagtctgacagagcgttgcacacagctggaaaaccagcgtgatgatgcccgtctggagacgatgggggagaaagaaacggtcgcggcactgcgtggtgaggctgaagccctgaagcgtcagaaccagtcactgatggcggcgctttcaggcaataaacagaccggtggccagaatgcg

SEQ ID NO: 10 - DNA, 918 bp, synthetic construct
  source 1..918: mol_type = other DNA
gtggaagtggctgaagaagtgaaggccgtctccgccgcgctgtccgaacgcatcacccagctggcgacagaactgaatgacaaggcggtccgggctgcagaacgccgggttgcggaagtcacgcgtgctgccggtgaacagaccgcacaggcagagcgggagctggccgacgccgcgcagacagtcgacgacctggaagaaaaactggttgaactgcaggacagatatgacagtttgacgctggcgctggagtcagaacgttcactgcgtcagcagcatgatgtggagatggcccagctgaaagagcgtcttgcggccgctgaagagaatacccgtcaggaagaggaacggtatcaggagcagaagacagtgctgcaggatgcgcttaatgcggagcaggcacagcacaaaaacacgcgggaagacctgcagaaacgactggagcaaatttctgtcgaagctaatgcgcgtacagaagaactgaagtctgaacgcgataaagtcaatactttccttacccgccttgaatcgcaggaaaatgcgctggcctcagaagaacagcagcatctggccacccgcgaaacgctgcagcaacgcctcgagcaggccatcgctgacacgcaggcgcgcgccggtgagattgcacttgaacgtgacagagtcagcagcctcaccgcaaggctggaatcgcaggaaaaggcctcctcggagcaactggtgcgtatgggcagtgaaatagccagtctgacagagcgttgcacacagctggaaaaccagcgtgatgatgcccgtctggagacgatgggggagaaagaaacggtcgcggcactgcgtggtgaggctgaagccctgaagcgtcagaaccagtcactgatggcggcgctttcaggcaataaacagaccggtggccagaatgcg

SEQ ID NO: 11 - DNA, 726 bp, synthetic construct
  source 1..726: mol_type = other DNA
gtgatccctgactacttcaagcagagcttccccgagggctacagctgggagcgcagcatgacctacgaggacggcggcatctgcatcgccaccaacgacatcacaatggagggggacagcttcatcaacaagatccacttccagggcacgaacttcccccccaacggccccgtgatgcagaagaggaccgtgggctgggaggccagcaccgagaagatgtacgagcgcgacggcgtgctgaagggcgacgtgaagatgaagctgctgctgaagggcggcggccactatcgctgcgactaccgcaccacctacaaggtcaagcagaagcccgtaaagctgcccgactgccacttcgtggaccaccgcatcgagatcctgagccacgacaaggactacaacaaggtgaagctgtacgagcacgccgtggccaagacttccaccgacagcatggacgagctgtacaagggtggcagcggtggcatggtgagcaagggcgaggagaccattacaagcgtgatcaagcctgacatgaagaacaagctgcgcatggagggcaacgtgaacggccacgccttcgtgatcgagggcgagggcagcggcaagcccttcgagggctctcagacgattgatttggaggtgaaggagggcgccccgctgcccttcgcctacgacatcctgaccaccgccttccactacggcaaccgcgtgttcaccaagtacccacgg

SEQ ID NO: 12 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgatatccagatcatatgaagcagcacgatttctttaaaagtgcgatgcccgagggttatgttcaggagcgcaccatcttcttcaaggacgacgggaattataagacccgcgctgaggttaagtttgaaggggatacccttgttaacagaattgagctgaagggtatagactttaaagaagatggcaatatattggggcacaagctcgaatataattacaacagtcataacgtatatatcatggctgacaagcaaaagaatggcataaaagtgaattttaagatacgacacaatatagaggatggtagcgtccagcttgcggatcattaccagcagaatacaccaattggtgacggcccagtactgctgccggataatcactatctcagtacccagagtgcattgtccaaagacccgaacgagaaacgcgatcacatggtactgctggaatttgtaacggctgctggaattacattgggaatggacgagttgtacaag

SEQ ID NO: 13 - DNA, 708 bp, synthetic construct
  source 1..708: mol_type = other DNA
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaag

SEQ ID NO: 14 - DNA, 327 bp, synthetic construct
  source 1..327: mol_type = other DNA
atgaaaggcgtgcaggtggagactatctccccaggagacgggcgcaccttccccaagcgcggccagacctgcgtggtgcactacaccgggatgcttgaagatggaaagaaatttgattcctcccgggacagaaacaagccctttaagtttatgctaggcaagcaggaggtgatccgaggctgggaagaaggggttgcccagatgagtgtgggtcagagagccaaactgactatatctccagattatgcctatggtgccactgggcacccaggcatcatcccaccacatgccactctcgtcttcgatgtggagcttctaaaactggaa

SEQ ID NO: 15 - DNA, 270 bp, synthetic construct
  source 1..270: mol_type = other DNA
gagatgtggcatgaaggcctggaagaggcatctcgtttgtactttggggaaaggaacgtgaaaggcatgtttgaggtgctggagcccttgcatgctatgatggaacggggcccccagactctgaaggaaacatcctttaatcaggcctatggtcgagatttaatggaggcccaagagtggtgcaggaagtacatgaaatcagggaatgtcaaggacctcacccaagcctgggacctctattatcatgtgttccgacgaatctcaaagcag

SEQ ID NO: 16 - protein, 167 aa, synthetic construct
ESNQSNNGGSGNAALNRGGRYVPPHLRGGDGGAAAAASAGGDDRRGGAGGGGYRRGGGNSGGGGGGGYDRGYNDNRDDRDNRGGSGGYGRDRNYEDRGYNGGGGGGGNRGYNNNRGGGGGGYNRQDRGDGGSSNFSRGGYNNRDEGSDNRGSGRSYNNDRRDNGGDG

SEQ ID NO: 17 - protein, 48 aa, synthetic construct
MNLVAQLENEVASLENENETLKKKNLHKKDLIAYLEKEIANLRKKIEE

SEQ ID NO: 18 - protein, 51 aa, synthetic construct
MARNAYLRKKIARLKKDNLQLERDEQNLEKIIANLRDEIARLENEVASHEQ

SEQ ID NO: 19 - protein, 306 aa, synthetic construct
VEVAEEVKAVSAALSERITQLATELNDKAVRAAERRVAEVTRAAGEQTAQAERELADAAQTVDDLEEKLVELQDRYDSLTLALESERSLRQQHDVEMAQLKERLAAAEENTRQRRERYQEQKTVLQDALNAEQAQHKNTREDLQKRLEQISVEANARTEELKSERDKVNTFLTRLESQENALASRRQQHLATRETLQQRLEQAIADTQARAGEIALERDRVSSLTARLESQEKASSEQLVRMGSEIASLTERCTQLENQRDDARLETMGEKETVAALRGEAEALKRQNQSLMAALSGNKQTGGQNA

SEQ ID NO: 20 - protein, 306 aa, synthetic construct
VEVAEEVKAVSAALSERITQLATELNDKAVRAAERRVAEVTRAAGEQTAQAERELADAAQTVDDLEEKLVELQDRYDSLTLALESERSLRQQHDVEMAQLKERLAAAEENTRQEEERYQEQKTVLQDALNAEQAQHKNTREDLQKRLEQISVEANARTEELKSERDKVNTFLTRLESQENALASEEQQHLATRETLQQRLEQAIADTQARAGEIALERDRVSSLTARLESQEKASSEQLVRMGSEIASLTERCTQLENQRDDARLETMGEKETVAALRGEAEALKRQNQSLMAALSGNKQTGGQNA

SEQ ID NO: 21 - protein, 242 aa, synthetic construct
VIPDYFKQSFPEGYSWERSMTYEDGGICIATNDITMEGDSFINKIHFQGTNFPPNGPVMQKRTVGWEASTEKMYERDGVLKGDVKMKLLLKGGGHYRCDYRTTYKVKQKPVKLPDCHFVDHRIEILSHDKDYNKVKLYEHAVAKTSTDSMDELYKGGSGGMVSKGEETITSVIKPDMKNKLRMEGNVNGHAFVIEGEGSGKPFEGSQTIDLEVKEGAPLPFAYDILTTAFHYGNRVFTKYPR

SEQ ID NO: 22 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 23 - protein, 236 aa, synthetic construct
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 24 - protein, 109 aa, synthetic construct
MKGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLE

SEQ ID NO: 25 - protein, 90 aa, synthetic construct [sequence omitted]